(19) United States
(12) Patent Application Publication
(10) Pub. No.: US 2007/0299034 A1
(43) Pub. Date: Dec. 27, 2007

(54) Cell wall derivatives, their preparation process, and use thereof

(76) Inventors: Marie-France Versali, Tilff (BE); Sandrine Gautier, Liege (BE); Jean-Michel Bruyere, Liege (BE); Fabienne Clerisse, Braives (BE); Aurelie Bornet, Lucinges (FR); Pierre-Louis Teissedre, Montpellier (FR); Jean-Max Rouanet, Saint Gely Du Fesc (FR)

Correspondence Address: CLARK & BRODY, 1090 VERMONT AVENUE, NW, SUITE 250, WASHINGTON, DC, 20005, US

(21) Appl. No.: 11/785,769

(22) Filed: Apr. 20, 2007

Related U.S. Application Data
(63) Continuation-in-part of application No. 10/504,046, filed on Jan. 28, 2005.
(63) Continuation-in-part of application No. PCT/FR06/50674, filed on Jul. 4, 2006.

(30) Foreign Application Priority Data
Apr. 21, 2006 (FR) 0651415
Jul. 4, 2005 (FR) 0507066
Feb. 12, 2002 (BE) 2002/0093

(51) Int. Cl. C12P 19/28; A61K 31/722; C08B 37/08; C12H 1/02

(52) U.S. Cl. 514/55; 426/422; 435/85; 536/20

(57) ABSTRACT

In a first aspect, the present invention relates to a method for isolating cell wall derivatives from fungal or yeast biomass. According to this method, chitin polymers or chitin-glucan copolymers can be obtained. In another aspect, the invention relates to a method for preparing chitosan from chitin. The invention further relates to chitin polymers, chitin-glucan polymers and chitosan polymers obtainable by the methods according to the invention. Moreover, the invention relates to the use of chitin polymers, chitin-glucan copolymers or chitosan polymers obtainable by the method according to the present invention in medical, pharmaceutical, agricultural, nutraceutical, food, textile, cosmetic, industrial and/or environmental applications, and in particular of chitin-glucan copolymers used as a technological additive for treating a food-grade liquid or in orally administered compositions.

This is a Continuation In Part of the U.S. application Ser. No. 10/504,046 filed on Jan. 28, 2005, and of PCT/FR2006/050674, filed on Jul. 4, 2006 designating the United States of America and claiming the priority of the French Patent Application number FR 0651415 filed on Apr. 21, 2006.

The invention relates to cell wall derivatives from biomass, preparation thereof, and methods using the same.

### FIELD OF THE INVENTION

The present invention relates to a method for isolating cell wall derivatives from fungal biomass, comprising polysaccharides, in particular purified copolymers of chitin and beta-glucan. The invention also relates to a method for preparing said cell wall derivatives, obtainable by the method according to the invention.

Moreover, the invention relates to purified chitin-glucan copolymers obtained by the method according to the present invention, and to their use in pharmaceutical, medical, agricultural, nutraceutical, food, textile, cosmetic, industrial and/or environmental applications.

In one embodiment, the invention relates to the treatment of food-grade liquids and beverages with purified chitin-glucan copolymers.

In another embodiment, the invention relates to the use of purified chitin-glucan as food supplements to improve human and animal health and to prevent certain health disorders.

### BACKGROUND OF THE INVENTION

Natural polysaccharides such as starch, cellulose or chitin are of great technological importance, as there are available in massive amounts, and as they present unique characteristics often not found for synthetic polymers. Cells walls of fungi are organized by a network of polysaccharides, proteins, lipids, the major part of the insoluble fraction of cell walls being polysaccharides, namely chitin and beta-glucan. Several types of fungi are available as industrial co-products, like Aspergillus sp. (production of citric acid, proteins), or as food and food co-products, like Agaricus bisporus and Lentinus edodes.

Chitin is a natural high molecular weight polymer widely found in nature, in fact the second major biopolymer after cellulose. Chitin is a polysaccharide whose structure is close to that of cellulose. It is the main component of insect and crustacean cuticule, and is also part of the cell walls of some fungi and other organisms. Chitosan is produced at the industrial level by chemical modification of chitin, and is naturally found in a few organisms. Chitin is a polysaccharide comprising N-acetyl-D-glucosamine repeating units, linked through alpha(1,4) osidic bonds (as represented in Formula I), when it is extracted from shellfish shells (shrimps, lobsters, crabs) and fungi, and through beta(1,4) osidic bonds when it is extracted from squid pens and diatomeas. Beta-glucan is a polysaccharide comprised of D-glucose bonds, linked through beta osidic bonds, mainly through beta(1,3)(1,6) and beta(1,3) bonds when it is extracted from fungi like Schizophyllum commune, Aspergillus niger, Lentinus edodes, Grifola frondosa, Sclerotinia sclerotiorum. In many fungi, the alkali-insoluble fraction of the cell walls is made of both chitin and beta-glucan closely associated, probably through covalent bonds, as described for Aspergillus fumigatus by Fontaine et al. [T Fontaine, C Simenel, G Dubreucq, O Adam, M Delpierre, J Lemoine, C E Vorgias, M Diaquin, J P Latgé. (2000) Molecular organization of the alkali-insoluble fraction of Aspergillus fumigatus cell wall. J Bio Chem 275:27594].

Similar to cellulose, chitin is a fibrous polysaccharide that has additional chemical and biological properties useful in many industrial and medical applications. Nevertheless, chitin is more difficult to extract, since it is usually found in its natural structure in which it is closely associated with other substances.

Chitosan can be prepared by partial hydrolysis of the acetyl groups of the N-acetyl-glucosamine units, so that the polymer becomes soluble in dilute solution of most acids. Chitosan can be derived from a polymer extracted from biomass, chitin. It is defined by two molecular characteristics, the average molecular weight and the degree of acetylation, that is the proportion of acetylated glucosamine units along the polymer backbone.

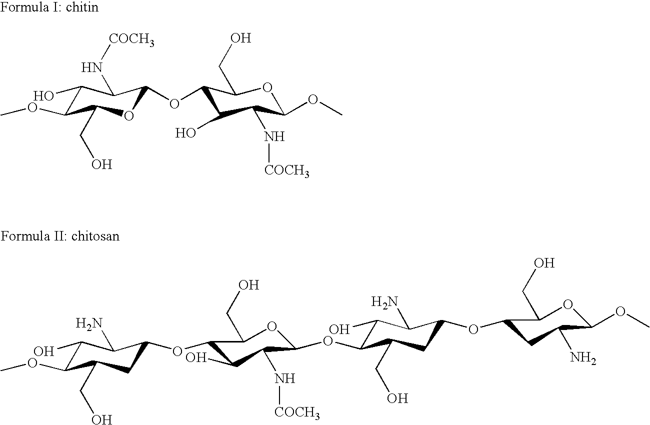

Industrial production of chitin and chitosan is generally exploiting wastes of crustacean shells, for instance crab or shrimp shells. Two steps, decalcification by acidic treatment and deproteneisation by alkaline treatment, allow chitin isolation, followed by a deacetylation step by using a hot concentrated alkaline solution. However chitin produced from crustacean biomass often contains high levels of minerals, mainly calcium carbonate, whose amount can reach up to 90% of chitin dry weight. The quality of chitin and chitosan is therefore often non reproducible and dependent on seasonal variation and crustacean species. The deacetylation method is a degrading one, and chitosan is often of very variable molecular weight and degree of acetylation, which makes product development by users more difficult. Moreover, high production costs result from the requirement of a huge calorific energy, and of large amounts of sodium hydroxyde, as well as the extensive acidic treatment required by the separation of chitin from calcium carbonate, whose amount can reach up to 90% of chitin dry weight.

Alternative sources for chitin and chitosan however do exist, like for instance fungi whose cell walls can contain up to 40% of the wall dry weight. The fungal mycelium is a complex network of filaments made of cells. The mycelium cell walls are made of hemicellulose, chitin and β-glucans. Fungi which contain sufficient amounts of chitin can be selected and grown specifically for the extraction of chitin. Furthermore, by-products of industrial fermentation process, such as the biomass collected after fungi or yeasts fermentation, also contain chitin associated with other biopolymers, mainly glucans, mannans, proteins and lipids. These fermentation by-products are generally burnt right after separation from the culture medium, because their storage is not economically relevant.

For chitin and chitosan to be used in as many applications as possible, their quality should be uniform and pure. The production of chitosan from a pure chitin, which would be available in large amounts in a reproducible way and would contain low amounts of inorganic and protein impurities would therefore be a substantial progress in this field.

The state of the art regarding alternative sources of chitin and chitosan to the crustacean ones is not very wide. A few patent and patent applications refer to fungal mycelium as a potential industrial source of chitin, for instance patents U.S. Pat. No. 4,960,413, No. 6,255,085, No. 4,195,175, No. 4,368,322, No. 4,806,474, No. 5,232,842, No. 6,333,399, and patent applications WO 01/68714, GB-A-458,839, GB-A-2,026,516, GB-A-2,259,709, DE-A-2,923,802 et RU-C-2,043,995. Most of these documents disclose methods for preparing chitosan or chitosan-glucan from fungal mycelium. Moreover, the methods describe direct transformation of chitin contained in the fungal cell walls, without any intermediate step for the isolation and purification of chitin. Therefore the methods described in these patents and patent applications do not allow the isolation of pure chitin as a source of pure chitosan. In these methods, highly concentrated alkaline solutions and severe temperature and duration conditions are employed, which again bring high pollution risks. Furthermore, these aggressive processes probably yield very low molecular weight chitin derivatives and chitosan, and cannot be used for the production of higher molecular weight chitosan.

Other articles describe fundamental studies of the cell wall structure of some fungal species, for example, Hartland et al. (1994) Yeast 10, 1591-1599; Hong et al. (1994) Yeast, 10, 1083-1092; Hearn et al. (1994) Microbiology 140, 789-795; Fontaine et al. (2000) Journal of Biological Chemistry 275, 27594-27607. These studies consistently conclude that the cell walls are made mainly of chitin and beta-glucans, and that the two types of polymer chains are closely associated, probably through covalent bonds in most fungi. Some of these studies mention the use of specific enzymes to selectively degrade the components of the cell walls, namely glucanases and chitinases, in order to further identify residual sugars to be able to estimate the initial polysaccharide composition.

In the field of treating food-grade liquids, especially treating food-grade liquids obtained from plants, for instance fruit juices or fermented drinks, and in particular wines, champagnes, beers or ciders, it is known practice to treat the product to be obtained with technological additives in order to remove undesirable compounds, which are especially the cause of instability and of dietary risks, or to adjust its composition.

It is especially known practice to use compounds such as bentonite, kaolin, PVPP, a food-grade gelatin, a fish paste, casein and potassium caseinate, ovalbumin, lactalbumin, silicon dioxide in gel or colloidal solution form, etc., for treating food-grade liquids, such as those mentioned above.

Mycotoxins, and in particular ochratoxin A (OTA) and aflatoxins, are now systematically controlled in food and drinks since their toxic effects have been demonstrated (nephrotoxicity, neurotoxicity, immunodeficiency, suspected carcinogenicity). It is nowadays recommended not to exceed a daily dose of mycotoxins of 0.3-0.9 μg/day. Until the limit becomes set by a European directive, the Office International de la Vigne et du Vin (OIV) [International Office of Wine and Grape] recommends not exceeding a content of 2 μg/l in wines.

Laboratory- and vineyard-based experiments have moreover explored the biological control route, by means of Trichoderma, the antagonist fungus of Aspergillus carbonarius. Three times less contamination has been observed. However, the results really depend on the strain of OTA. The means for controlling the OTA amount essentially to prophylaxis at the vineyard, with the drawback of seeing pesticide residues and metabolites arise in the grapes and musts. Few solutions have emerged at the present time, especially in oenology. If the grapes are contaminated, then in vinification, the OTA content increases during maceration. The OTA content depends on the alcoholic degree. Alcohol is a solvent for the OTA molecule and dissolves it in wine. For red wine, thermovinification does indeed appear to be advantageous, although complementary studies on optimizing the heating of the grape harvest still need to be performed (heating time, temperature, flash vacuum-expansion). Microbiological investigations distinguish oenology disinfection products that are more efficient than others, but with very high costs and risks of nonselectivity (removal of the yeast/bacterial strains that are useful for alcoholic or malolactic fermentation). As regards the use of oenological additives such as silica gel, oenological charcoal, potassium caseinate, gelatin or bentonites, the results are not very conclusive since they remove very little OTA (apart from oenological charcoal and potassium caseinate) and lead to major drawbacks. All these products are liable to result in the appearance of allergenic residues, especially in musts and wines.

The use of oenological charcoal has the major drawback of removing all the phenolic compounds (anthocyans and tannins in particular). The phenolic compounds are essential as constituents that condition the color and the sensory perception of wines and other drinks obtained especially by fermentation.

Silica gels and gelatin are entirely inefficient as regards removing OTA and are normally used for performing clarification with the aid of tannins in order to clarify musts or wines (to remove proteins or to soften). Uses of excessively high doses of these oenological products have the major drawback of resulting in protein breakage in the case of gelatin and of leading to high risks of substantial loss of polyphenols as regards silica gels.

As regards bentonites, they are used for the clarification or protein stabilization operations on musts and wines, and bind certain unstable proteins to allow their removal. They are also capable of binding coloring matter. However, studies have shown that they release high levels of aluminum in musts and wines. A high input of aluminum into the food ration is liable to have public health repercussions regarding degenerative diseases.

Moreover, several constraints exist during oenological treatments on must or wine:

For the destaining of white musts and white wine, the use of oenological charcoal has the major drawback of removing all the phenolic compounds (anthocyans and tannins in particular). Appendix IV of EC regulation 1493/1999 provides for the treatment of white musts, new white wines still in fermentation and white wines with charcoals for oenological use, with certain limitations (§1 paragraph i and §3 paragraph o). Although the European Community regulation does not explicitly specify the purpose of this treatment, it can be performed only for destaining white vine-growing and wine-making products and must in no way be used to deodorize wines with an obvious poor taste. Specifically, it provides that the treatments can be performed only in order to allow good vinification or good storage of the products under consideration (Art. 42 of Rule EC 1493/1999). Thus, active charcoal is unsatisfactory for solving the technical problems posed below.

As regards the current iron-removal treatments of wines, the maximum copper content set by the OIV is 1 mg/l. For iron, the risk of iron breakage occurs at above a content of about 8 mg/l. The iron removal consists in removing the excess iron liable to cause iron breakage, which results in a cloudy appearance unfit for consumption. The presence of an excess of iron is often due to a vat in poor condition or to particles of earth present on the grapes during harvesting. The addition products for treated wines are potassium ferricyanide (white and rosé wine) and calcium phytate (red wine).

For treatment with potassium ferricyanide, there are nowadays technical, administrative and analytical constraints. In particular, the total removal of potassium ferricyanide must be controlled on the wine after treatment: this is long, expensive and meticulous with implications in terms of food safety and public health.

For treatment with calcium phytate, there are again constraints concerning the analytical and administrative controls of the treatment that are the responsibility of the oenologist:

treatment under the mandatory control of an oenologist,

after treatment, the wine should still contain traces of iron,

provisions regarding the control of the use of phytate decreed or to be decreed by each member state.

As regards the presence of heavy metals in wines, the maximum content of heavy metals in wines is governed by the OIV. The lead level has been set at 200 μg/l since 1996, and the cadmium level at 10 μg/l since 1981.

Treatment with potassium ferricyanide can also remove traces of heavy metals. It is also possible to remove major and heavy metals indirectly by means of electrodialysis or a cation-exchange resin. However, this process is complicated to implement and is not accessible to all producers, since it is expensive. Moreover, this process is not authorized in all countries.

Moreover, purity criteria for the technological additives in oenology are established. Oenological products are manufacturing additives or additives. In this respect, they should satisfy the purity criteria defined by the regulation when such is the case. Certain products are not, are no longer or are poorly defined this is the case, for example, for charcoals and tannins.

During vinification, clarification, stabilization, specific treatments, storage or filtration operations, many oenological, additive and media-filtering products, or specific treatments are used, and products for curative purposes are generally involved above all, making it possible to overcome certain problems during the élevage of wines. The following are mainly encountered:

iron-removing products, for instance potassium ferricyanide for white wines, and phytate-based Afferol for red wines;

products intended to remove oxidation products, for example soluble casein, Casei+(potassium caseinate), Polylact (PVPP, Casein) or Viniclar (PVPP));

bentonites to remove any excesses of proteins, for example powdered or granulated Microcol.

The agents permitted for treating food-grade liquids are known to those skilled in the art and are referenced by the national legislations, for instance the agents authorized to treat wines and fruit juices in the USA (27CFR24.246) or in Europe (EC Rule 1493/1999 and EC 1622/2000).

Among these compounds, some of them are unsuitable for treating various types of food-grade liquids, for instance various wines, various beers, various champagnes, etc., or are unsuitable for withdrawing the various compounds to be removed.

The technological additives mentioned above should be used relatively specifically as a function of the beverage to be treated. Thus, for example, for two different wines, it will be necessary to use different technological additives during the treatment. For example, to produce a white wine, clarification of the must will be performed with bentonite or fish paste after pressing in order to remove must deposits and proteins. On the other hand, in the case of a red wine, PVPP may be used, which binds the polyphenols of the wines, for example to produce young primeur wines.

Similarly, for two different steps of the process of treatment of the same beverage, it will be necessary to use two different technological additives, which especially poses problems of storage, labeling and use. For example, removal of the oxidation products is performed with casein or PVPP, but removal of the coloring phenolic matter is performed with oenological charcoal, and pectolytic enzymes are used to degrade pectins. Bentonite is not used, for example, at the present time for treating the finished product (storage, bottling or élevage).

Moreover, the known technological additives have the risk of deteriorating the organoleptic properties, which is detrimental to the finished beverage, in particular as regards beverages obtained from plants, for instance beers, wines, champagnes, ciders and fruit juices.

It is known practice especially from patent applications FR 2 599 048 and EP 0 501 381 to use chitosan for treating food-grade liquids of plant origin. The teaching of Spagna et al. (Spagna Giovanni et al. “The stabilization of white wines by absorption of phenolic compounds on chitin and chitosan”, Food research International, Applied Science, Barking, Vol. 29, No. 3-4, 1996, pages 241-248) also describes the removal of the polyphenols from a white wine by using chitosan. The use of chitin is also described, but, according to said article, is not suitable for removing polyphenols. However, the use of chitosan has the drawback that almost all the commercially available chitosan is of animal origin, and thus presents risks of allergies. Commercially available chitosan is mainly derived from the shell of crustaceans (shrimp, crab or lobster). Specifically, chitosan is a polysaccharide that has demonstrated its capacity to clarify and stabilize food-grade liquids, and that is commercially available, but only for home and non-industrial uses as an additive for the home manufacture of wine and beer. The use of this chitosan of animal origin as a technological additive for the treatment and stabilization of food-grade liquids poses at least two problems. On the one hand, technological additives of animal origin are not in favor with the majority of producers of food-grade liquids, and should or will have to be systematically cited on the labeling as stipulated by the legislations in force or under preparation. On the other hand, extracts of crustaceans are not recommended for people who are allergic to crustaceans, who are warned on the labeling. It should be borne in mind that allergy to crustaceans is one of the commonest allergies (3% of adults in the USA according to a recent study).

Patent application WO 98/17386 concerns a method for removing only the pesticides from fruit juices by especially using chitin or chitosan derivatives. However, in this case also, the compounds used and described by the invention are of animal origin.

Patent application WO 98/17386 concerns a method for removing pesticides and/or agricultural chemicals from food-grade liquids and non-foodstuffs. Reference is made in said application to chitin and chitosan, but no example is given concerning these compounds. The compounds used in the examples of said patent application concern derivatives of the type such as alkyl esters or aryl esters of chitin, of polysaccharides or of chitosan. Although these hydrophobic compounds of the octanoyl- or benzoyl-chitin type allow the removal of pesticides, which are molecules of lipophilic nature, it is not obvious, without additional experience, that chitin alone would have made it possible to remove the pesticides. Moreover, it is not possible to extrapolate the treatment described in said patent application to the removal of other molecules such as proteins, polyphenols, mycotoxins, metals, etc., which molecules are present in food-grade liquids of plant origin. Thus, said document does not describe a technological additive that allows the treatment of food-grade liquids of plant origin without substantially deteriorating their organoleptic properties.

Patent application DE 198 10 094 (U.S. Pat. No. 6,402,953) describes the use of chitin or chitosan of fungal origin for treating radioactive contaminants of aqueous solution, especially for removing therefrom heavy metals such as cesium, uranium, plutonium, etc. Said patent application is thus very remote from the technical field of the present invention, which concerns the treatment of food-grade liquids of plant origin. Moreover, in the light of the process for obtaining the “absorbent” described in the examples, the chitin-based material of fungal origin described in said document is not pure, in the sense that it runs the risk of resulting in the release of residues that are soluble in the treated food-grade liquid, which runs counter to the objective of the present invention. This impure compound would allow water to be treated, but would not really be suitable for treating food-grade liquids of plant origin, which especially comprise proteins, polyphenols, metals or mycotoxins, in order especially to conserve and/or to not impair their organoleptic properties.

Thus, the prior art cannot provide a technological additive for treating food-grade liquids of plant origin, since either the additive has the risk of substantially deteriorating the organoleptic properties by releasing residues, or it has the risk of impairing the organoleptic properties by removing beneficial compounds, or it is unsuitable for food use because it is of animal origin, which is generally undesirable.

It is further known that both microscopic and edible fungi have properties that are beneficial to the health, such as hypoglycaemic, blood-cholesterol lowering, antioxidant or immunostimulant properties. Fungi contain digestible compounds that have important properties with respect to the health of the gastrointestinal tract and of the body in general, and are defined as being polysaccharide-type dietary fibre. They have the property of acting on glucose circulation by lowering the blood insulin concentration, increasing the viscosity of foods in the small intestine and slowing down carbohydrate absorption. They therefore have the ability to regulate sugar metabolism. They also play a role in the regulation of arterial blood pressure. Fungi which contain large amounts of fibre have a beneficial effect on reducing the concentration of total cholesterol, and of HDL-cholesterol in the blood, as shown by a study by Fukushima et al., (2000) fibre from the mushroom Agaricus bisporus (Fukushima M, Nakano M, Morii Y, Ohashi T, Fujiwara Y & Sonoyama K (2000) J. Nutr. 130:2151). They therefore play an indirect role in the prevention of hypertension and of cardiovascular diseases and, more largely, on obesity and metabolic syndrome.

Very few foods exert immunostimulant properties. Most food products induce immunodepression rather than immunostimulation. Fungi such as the shiitake mushroom (Lentinus edodes), the maitake mushroom (Grifola frondosa), the reishi mushroom (Ganoderma lucidum) or the ABM mushroom (Agaricus blazei murill) are “immunostimulant” foods. The immunostimulant function is largely attributed to the presence of beta-glucan type polysaccharides present in their cell walls (Lull C, Wichers H J & Savelkoul H F J (2005) Antiinflammatory and immunomodulating properties of fungal metabolites. Mediators Inflammation 2:63).

Certain plant fibre is recommended for preventing, inhibiting or treating obesity and obesity-related diseases, for instance oligofructoses derived from chicory inulin, or laminarin, a beta-glucan derived from algae. Oligofructoses act by fermenting in the colon, thus releasing compounds capable of suppressing the plasma content of ghrelin, a hormone that usually stimulates the appetite. Indirectly, by increasing satiety and reducing food intake, an effect on cholesterol level and atherosclerosis is observed, among the numerous effects associated with metabolism syndrome and with cardiovascular diseases.

Chitosan is also known to exert a blood-lipid-lowering and blood-cholesterol-lowering action. These effects are attributed to a mechanism of interaction between the dietary fatty acids in the stomach or the bile acids (negatively charged), and chitosan, which is positively charged. However, chitosan has the drawback that its source is shellfish, which is potentially allergenic.

Non-food-related uses of compositions containing the chitin-glucan copolymer are known, in particular as an active agent for healing the skin. The mycoton and mycoran compositions derived from Aspergillus niger are described for their properties when applied to the skin and to scars. However, no oral application is known, in particular in the dietary or pharmaceutical field.

Most of the studies carried out on the beneficial effects of fibre of fungal origin were done so either on fresh fungi, or on powdered fungi, or on beta-glucans that are soluble in an aqueous medium, generally extracted from plants. These products are not, however, the most suitable for the above-mentioned indications.

### GOALS OF THE INVENTION

It is in general an object of the present invention to provide an improved industrial method for the isolation of cell wall derivatives from fungal or yeast biomass. It is in particular an object of the present invention to provide an industrial method for isolating chitin polymers or chitin-glucan polymers. It is another object of the present invention to provide an industrial method for preparing chitosan.

Another object of the invention is to isolate chitin polymers and to prepare chitosan following a rapid process that does not require high-energy consumption nor chemicals that would be detrimental to the environment.

Another aspect of the invention is to provide a method to isolate pure chitin polymers and to prepare chitosan polymers from non-animal origin, which are suitable for applications in various fields.

The present invention also aims to provide polymers of chitin having a high degree of purity. Moreover, it is another object of the present invention to provide chitin-glucan copolymers wherein the amount of chitin and beta-glucan is adjustable. The present invention further aims to provide chitosan having a high degree of purity and a controllable degree of acetylation and molecular weight.

Both chitin and beta-glucan exert important technological, physico-chemical and biological properties. It is the goal of the invention to provide chitin-beta-glucan copolymers, obtained by the method of the invention by extraction and purification from industrial fungal co-products.

One aim of the invention is also to solve the technical problem that consists in providing a technological additive for stabilizing finished food-grade liquids, while at the same time preserving their organoleptic properties.

An aim of the invention is to solve the technical problem that consists in providing a technological additive for decontaminating finished food-grade liquids, especially for obtaining impurity contents below the levels defined by the legislation in force, in particular for wines, champagnes, ciders and beers.

An aim of the invention is to solve the technical problem that consists in providing a technological additive for clarifying finished food-grade liquids.

An aim of the present invention is especially to solve the problems defined above, especially as regards the treatment of wines, red wines and/or white wines and/or rose wines and/or natural sweet wines.

An aim of the present invention is also to provide a single technological additive for performing various steps of the treatment of a beverage, and preferably of wine, champagne, cider or beer. The aim of the present invention is also to provide a technological additive that can be used for different wines, champagnes, ciders, beers, etc.

Another main objective of the invention is to provide a family of natural substances of fungal origin that makes it possible to provide a food supplement or a pharmaceutical composition, in particular for improving human and animal health as a supplement to a balanced diet and good hygiene practice, in particular in the prevention and/or combating of pathologies such as metabolic syndrome, obesity, diabetes and cardiovascular diseases, or related diseases.

An objective of the present invention is also to provide a family of substances that inhibits impeccable food safety, while at the same time being readily available in large volume at a cost compatible with use as food supplements.

An objective of the invention is also to provide a family of natural substances of non-animal origin and of excellent purity, that are well characterized and with good traceability.

An objective of the present invention is also to provide a food supplement, of polysaccharide type, that is stable and easy to formulate.

An objective of the invention is to propose a pharmaceutical active agent or food supplement that makes it possible to return the parameters associated with the metabolic syndrome, obesity, diabetes and/or cardiovascular disease pathologies to the normal level, such as, by way of example, the triglyceride content, the balance between LDL-cholesterol and HDL-cholesterol, the surface area of the aortic arch covered with lipid deposits, the antioxidant capacity of plasma, etc.

Finally, the aim of the present invention is to solve all the technical problems mentioned above in a reliable, inexpensive and industrially usable manner, and especially less expensively and more viably than by using a non-animal chitosan.

### SUMMARY OF THE INVENTION

The present invention relates to a method for isolating cell wall derivatives from fungal biomass, comprising polysaccharides, in particular purified copolymers of chitin and beta-glucan. The invention also relates to a method for preparing said cell wall derivatives.

Moreover, the invention relates to purified chitin-glucan copolymers obtained by the method according to the present invention, and to their use in pharmaceutical, medical, agricultural, nutraceutical, food, textile, cosmetic, industrial and/or environmental applications.

In one embodiment, the invention relates to the treatment of food-grade liquids and beverages with purified chitin-glucan copolymers.

In another embodiment, the invention relates to the use of purified chitin-glucan as food supplements to improve human and animal health and to prevent certain health disorders.

### DETAILED DESCRIPTION OF THE INVENTION

In a first aspect, the present invention relates to a method for isolating cell wall derivatives from fungal or yeast biomass comprising the subsequent steps of:
a) contacting said biomass with a basic solution, whereby an alkali-soluble fraction and an alkali-insoluble fraction are obtained and whereby said alkali-soluble fraction is discarded and said alkali-insoluble fraction comprising said cell wall derivatives is retained,
b) contacting said alkali-insoluble fraction with an acidic solution, by suspending said alkali-insoluble fraction and bringing said suspended fraction into contact with said acidic solution in order to obtain a suspension of acidified alkali-insoluble fraction comprising said cell wall derivatives, and optionally
c) contacting said acidified suspension of alkali-insoluble fraction with β-glucanase enzymes whereby said cell wall derivatives are obtained.

The fungal or yeast biomass treated in the present method according to the invention is made of fungi or yeast cells, of which the cell walls contain chitin. Alternatively, said biomass may also be a side-product of an industrial cultivation process wherein a fungal or yeast culture is used.

The invention provides a method that avoids the main drawbacks of existing methods. More particularly, the invention provides a chitin isolation method with economical and environmental advantages over existing methods and sources. More particularly, the invention discloses a method that allows separating chitin from β-glucans in a controlled way, without degradation or transformation of the chitin chains.

In a preferred embodiment, the invention relates to a method wherein said cell wall derivatives obtained in step c) are chitin polymers or chitin-rich chitin-glucan copolymers. More in particular, the invention relates to a method for the isolation of chitin from fungal or yeast biomass in order to obtain chitin polymers, essentially free of other polysaccharides like β-glucans.

The term “chitin polymers” refers to chitin polymers that contain more than 80% of chitin and less then 20% of beta-glucan, and preferably more than 90% of chitin and even more preferred more than 95% chitin.

The term “chitin-rich chitin-glucan copolymers” refers to polymers, which comprise chitin polymers as well as glucan polymers in certain relative amounts, but having a higher relative amount of chitin than of glucan. The method according to the invention enables to specifically adjust the amounts of chitin and glucan in these chitin-glucan copolymers. The amount of chitin in the copolymers can be adjusted by controlling the conditions of the enzymatic hydrolysis step in the present method. The invention thus provides a method for obtaining copolymers comprising chitin with a controllable purity. The term “polymers comprising chitin with controllable purity” refers to a polymer product wherein the amount of chitin can be adjusted in a controllable way by means of glucanase enzymes. In a preferred embodiment, the amount of chitin in said chitin-rich chitin-glucan copolymers is adjustable and preferably higher than 75% and even more preferably higher than 80%.

In another preferred embodiment, the invention relates to a method wherein said cell wall derivatives obtained in step a) or b) are chitin-glucan copolymers, from which the relative amounts of chitin over glucan depend on the used biomass. The term “chitin-glucan copolymers” as used herein refers to copolymers obtained after extraction of fungal or yeast biomass but before enzymatic reaction by means of glucanase enzymes and refers to copolymers obtained in the alkali-insoluble part of the fungal biomass after treatment. The amount of chitin in said chitin-glucan copolymers is defined by the organism from which it is extracted. In a preferred embodiment, mycelium of Aspergillus niger is used in the method according to the invention, and chitin-glucan copolymers extracted from the mycelium of Aspergillus niger comprise between 30 and 50% (w/w) of chitin and between 50 to 70% of beta-glucan.

The terms “chitin” and “chitin polymers” are used herein as synonyms. In addition, the terms “chitosan” and “chitosan polymers” are used herein as synonyms.

In a second aspect, the present invention relates to a method for preparing chitosan from chitin comprising the subsequent steps of:
a) contacting said chitin with a basic solution, whereby an alkali-soluble fraction and an alkali-insoluble fraction is obtained and whereby said alkali-soluble fraction is discarded and said alkali-insoluble fraction comprising partially deacetylated chitin is retained,
b) contacting said alkali-insoluble fraction with an acidic solution, by suspending said alkali-insoluble fraction and bringing said suspended fraction into contact with said acidic solution in order to obtain an acidified alkali-insoluble fraction comprising said partially deacetylated chitin, and
c) contacting said acidified fraction with a chitin deacetylase, whereby chitosan is obtained.

In this aspect, the invention provides a method for preparing chitosan, whereby high molecular weight chitosan, with controlled degree of acetylation, by an enzymatic deacetylation reaction of chitin with a chitin deacetylase enzyme. In this aspect, the invention also provides a method for preparing chitosan whereby a low and medium molecular weight chitosan with a controllable degree of acetylation can be obtained.

The term “low and medium molecular weight” refers to an average molecular weight lower than 100 kDa, as measured by Ubbelohde capillary viscosimetry. The term “high molecular weight” refers to an average molecular weight higher than 100 kDA, as measured by capillary Ubbleohde viscosimetry.

The term “chitosan having a controlled degree of acetylation” refers to a product wherein the degree of acetylation, that is the proportion of N-acetyl-glucosamine units, can be adjusted in a controllable way.

In a preferred embodiment, the invention relates to a method wherein said chitin is fungal or yeast chitin obtainable by the method for isolating cell wall derivatives from fungal or yeast biomass according to the present invention. Since this source of chitin comprises a very high degree of purity, the invention allows preparing chitosan, which also yields a high degree of purity. In addition, this method also provides chitosan having an adjustable degree of acetylation, since the degree of acetylation can be adjusted by controlling the conditions of the enzymatic deacetylation in the present method.

In another aspect, the invention relates to a method for preparing chitosan from fungal or yeast chitin comprising the subsequent steps of:
a) contacting said chitin with a basic solution, whereby an alkali-soluble fraction and an alkali-insoluble fraction is obtained and whereby said alkali-soluble fraction is discarded and said alkali-insoluble fraction is retained,
b) contacting said alkali-insoluble fraction with an acidic solution, by suspending said alkali-insoluble fraction and bringing said suspended fraction into contact with said acidic solution whereby an acid-insoluble fraction and an acid-soluble fraction is obtained and whereby said acid-insoluble fraction is discarded and said acid-soluble fraction comprising chitosan is retained.

In this aspect, the invention provides a method for preparing chitosan, which yields low and medium molecular weight. This method comprises an alkaline hydrolysis reaction of chitin obtained from fungal or yeast biomass. The term “low and medium molecular weight” refers to an average molecular weight lower than 100 kDa, as defined above.

In another aspect, the present invention relates to chitin polymers obtainable by the method described above.

In addition, the invention also relates to chitin-rich chitin-glucan copolymers obtainable by the method described above.

The invention further relates to chitosan polymers obtainable by the method according to the method described above.

The invention further relates in another aspect to a composite material comprising chitin polymers, chitin-glucan copolymers, chitin-rich chitin-glucan copolymers, or chitosan polymers obtainable by the method according to the present invention.

In another aspect, the invention relates to the use of chitin polymers, chitin-rich chitin-glucan copolymers, chitin-glucan copolymers, or chitosan polymers obtainable by the methods described above in medical, pharmaceutical agricultural, nutraceutical, food, textile, cosmetic, industrial and/or environmental applications.

Method for Isolating Cell Wall Derivatives

The invention discloses in a first aspect a method for isolating cell wall derivatives from fungal and yeast biomass comprising the steps as described above.

In a preferred embodiment, the invention relates to a method characterized in that said cell wall derivatives are chitin polymers. The term “polymers” as used herein refers to high molecular weight substances that are mixtures of chains made by the repetition of one or several types of monomeric units. Generally, polymers are made of at least three monomeric units. The monomeric unit is the repeating unit that constitutes the polymeric chains. The term “chitin polymers” refers to a polymer made of at least 3 monomeric repeating units of β(1,4)-N-acetyl-(D)-glucosamine, and preferably more than 10, and even more preferably more that 20 monomeric units. Chitin polymers are chains of monomeric β(1,4)-N-acetyl-(D)-glucosamine units linked through a covalent β(1-4) osidic bond.

The present invention provides a method, which enables extracting chitin contained in the mycelium of fungi and yeasts. Prior art has repeatedly shown that in the cell walls of most yeast and fungi, chitin is associated with other polymers through covalent bonds, for example with polysaccharides of the β-glucans type, thereby forming a typical fibril structure. That is the reason why chitin is difficult to extract from the fungal and yeast biomass and to collect in a pure form. In order to obtain chitin chains, the chitin chains need to be separated from the other polymeric chains, preferably by a non-degrading method. The present invention discloses a method that allows separating chitin from other polymers, which comprise mainly β-glucans, without degradation of the chitin chains.

Chitin and chitin-glucan copolymers can be obtained from non-animal biomass, in particular from the cell walls of fungal mycelium or yeasts from several groups, including Zygomycetes, Basidiomycetes, Ascomycetes and Deuteromycetes and/or mixtures thereof, and preferably Ascomycetes. Aspergillus and yeasts like Saccharomyces belong to the latter group. In a preferred embodiment, the invention relates to a method characterized in that said biomass is selected from the group comprising but not limited to filamentous fungi or yeasts such as Aspergillium, Penicillium, Trichoderma, Saccharomyces, and Schizosaccharomyces species, and edible mushrooms such as Agaricus, Pleurotus, Boletus, and Lentinula species, and/or mixtures thereof. The invention includes genetically modified organism (GMO).

A common feature of these fungi and yeasts is the presence of chitin in their cell walls. In an even more preferred embodiment, said biomass is obtained from Aspergillus niger.

In another embodiment, the method is characterized in that said biomass is a side-product obtainable in a cultivation process wherein a fungal or yeast culture is used. Fungal mycelium can be collected in fungal cultures engineered for the industrial production of compounds like for example citric acid, enzymes, and antibiotics. Chitin and chitin-glucans copolymers can be extracted from cell walls of these cultivation side-products. In a preferred embodiment, said method is characterized in that said biomass is a side-product of a cultivation process wherein an Aspergillus niger culture is used for obtaining citric acid.

The method according to the invention comprises contacting said biomass with a basic solution, whereby an alkali-soluble fraction and alkali-insoluble are obtained and whereby said alkali-soluble fraction is discarded and said alkali-insoluble fraction comprising said cell wall derivatives is retained. The alkaline solution used to digest the fungal or yeast biomass is an aqueous solution of an alkali like sodium hydroxyde, potassium hydroxyde, ammonium hydroxyde, and preferably sodium or potassium hydroxyde. In a preferred embodiment, said basic solution comprises a concentration lower than 10% (w/v). The alkali concentration is preferably ranging between 0.1 and 15% (w/v), and is more preferably lower than 10%. The reaction is performed at a temperature preferably ranging between 5 and 120° C., and more preferably at a temperature lower than 60° C. The biomass is reacted in suspension in the alkaline solution at a concentration preferably ranging between 1 and 15% (dry weight, w/v), and more preferably between 3 and 12%. The reaction is preferably performed for 4 to 48 hours, and more preferably for less than 30 hours. This first extraction step allows to eliminate alkali-soluble compounds, including pigments, proteins, some lipids, and some polysaccharides.

In another preferred embodiment, the biomass can be treated in a first alkaline solution, filtrated and treated again in a second alkaline solution. Additives can be used in the alkaline suspension to improve the extraction of the alkali-insoluble product. Such additives may comprise but are not limited to organic solvents such as cyclohexane, ethyl acetate, methanol or ethanol; anti-foaming agents such as structol; tensio-active agents such as sodium dodecyl sulfate, poly(vinyl alcohol), tween or poloxamers; or enzymes preparations containing carboxylesterase, carboxylic ester hydrolase or triacylglycerol lipase (all synonym to EC 3.1.1.3).

For the isolation of the alkali-insoluble product of the biomass cell walls, which is a chitin-glucan copolymer in many fungal and yeast biomass, the first step is followed by repeated washing steps in water, followed by filtration and drying. For the isolation of chitin polymers, this first step is followed by repeated washing in water, followed by the further steps in the method as described below.

A second step in the method according to the invention comprises contacting said alkali-insoluble fraction with an acidic solution, by suspending said alkali-insoluble fraction and bringing said suspended fraction into contact with said acidic solution in order to obtain a suspension of an acidified alkali-insoluble fraction comprising said cell wall derivatives.

After a last filtration step as explained above, the alkali-insoluble product is suspended in water in order to obtain a concentration preferably between 1 and 8% (w/v), and more preferably between 1 and 5%. Then the pH of the aqueous suspension of the alkali-insoluble product is adjusted below 7.0 by addition of an acidic solution. The acidic solution is preferably an aqueous solution of an acid, for instance chlorhydric, acetic, formic, lactic, glutamic, aspartic, or glycolic acid, and preferably acetic acid. This step is preferably performed at a temperature between 5 and 60° C., more preferably below 30° C.

An optional third step in the method according to the invention comprises contacting said acidified alkali-insoluble fraction with β-glucanase enzymes whereby said cell wall derivatives are obtained. In a more preferred embodiment, the method is characterised in that the β-glucanase enzymes are selected from the group comprising endo-β(1,3)-glucanase, exo-β(1,3)-glucanase, β(1,3)(1,4)-glucanase, β(1,6)-glucanase enzymes, or any mixture thereof. Even more preferred, a mixture of enzymes is added to the suspension of the acidified alkali-insoluble fraction, in order to hydrolyse the β-glucan chains that are associated with chitin. β-glucanase enzymatic activities can be easily found in commercial preparation of β-glucanases supplied by several companies. The hydrolysis reaction is preferably performed at a temperature between 5 and 60° C., and more preferably below 40° C. The reaction duration is preferably below 5 days. Preferred preparations contain mainly β-glucanase activities, and preferably low or no chitinase activity. Commercially available enzyme preparations can be used, extracted from organisms like for example Bacillus subtillis, Arthrobacter luteus, Penicillium emersonii; Penicillium funicolosum, Humicola insolens, Aspergillus niger, Trichoderma harzanium, Trichoderma longibrachiatum. Said preparations are available from companies like NovoZymes, Erbsloh, Roche or Lyven. In order to hydrolyse the β-glucans chains of polysaccharides extracted from the cell walls of the fungal mycelium, preferred enzymatic preparation are those which contain the following β-glucanase activities: endo-β(1.3-1.4)-glucanase (EC 3.2.1.6); endo-β(1.3)-glucanase (EC 3.2.1.39); exo-β(1.3)-glucanase (EC 3.2.1.58); endo-β(1.6)-glucanase (EC 3.2.1.75); and/or β-glucosidase (EC 3.2.1.21, β-D-glucoside glucohydrolase). In example 3, provided below, several commercial enzymatic preparations are illustrated for use in the method according to the invention.

In a preferred embodiment the invention relates to a method characterized in that said cell wall derivatives are chitin-rich polymers (i.e. chitin polymers or chitin-rich chitin-glucan copolymers). The insoluble fraction obtained in the method mainly contains macromolecular chains of chitin, linked with a certain amount of residual oligomeric or macromolecular chains of β-glucan. The ratio of chitin to glucan can easily be adjusted by controlling the conditions of the reaction, mainly by the β-glucanase preparation employed and by the reaction duration. In a more preferred embodiment, the invention relates to a method characterized in that the relative amount of chitin is adjustable and preferably higher than 80%, and more preferably higher than 90% and even more preferably higher than 95%. The relative amount of chitin can be measured by solid-state ¹³C-NMR. Said chitin-rich insoluble fraction can also contain residual proteins, lipids and carbohydrates.

In another aspect, the present invention relates to purified chitin-glucan copolymers obtainable by the method according to the present invention.

The ratio of chitin to glucan may be comprised between 5:95 and 95:5, preferably between 20:80 and 70:30, (w/w).

In one embodiment, the mycelium of Aspergillus niger is used in the method according to the invention, and chitin-glucan copolymers extracted therefrom comprise between 30 and 50% (w/w) of chitin and between 50 and 70% of beta-glucan. In another embodiment, Lentinus edodes is used in the method according to the invention, and chitin-glucan copolymers extracted therefrom comprise between 30 and 50% (w/w) of chitin and between 50 and 70% of beta-glucan.

In a preferred embodiment, the chitin-glucan copolymer contains less than 10% of water-soluble compounds.

In another preferred embodiment, the chitin-glucan copolymer has a purity above 80. In another preferred embodiment, the chitin-glucan copolymer has a purity above 85%. In another embodiment, the chitin-glucan copolymer contains less than 2% of lipids, and preferably less than 1% of lipids.

In another embodiment, the chitin-glucan copolymer contains less than 40 mg/kg of heavy metals, and preferably less than 20 mg/kg of heavy metals, as determined by the method described in the 2.4.8F monography of the European Pharmacopeia. In another preferred embodiment, the chitin-glucan copolymer contains less than 2 ppm of arsenic, as determined by AES-ICP. In another preferred embodiment, the chitin-glucan copolymer has a microbiogical quality suited to food-related applications, preferably less than 10,000 cfu/g for both total microbial count and yeast and molds, more preferably less than 1,000 cfu/g.

In a preferred embodiment, the ratio of chitin to beta-glucan is comprised between 30:70 and 50:50 (w/w), as determined by solid-state 13C NMR.

Optionally, in a further step of the present method, a second alkali solution is added at the end of the hydrolysis reaction, for example a solution of sodium hydroxide, potassium hydroxide, ammonium hydroxide, and preferably sodium or potassium hydroxide. This further step is preferably carried out at a temperature between 20 and 80° C., and more preferably below 70° C., preferably for a duration of 30 minutes to 3 hours, and more preferably below 2 hours. This second alkaline treatment allows the separation of chitin and β-glucan to be completed, thereby isolating chitin.

For the production of chitin polymers, the process is preferably continued with repeated washing steps, followed by a drying step.

In another aspect, the present invention relates to chitin polymers obtainable by the method according to the present invention. There are several advantages to the method disclosed in the invention. The method allows extracting pure chitin, partially or totally separated from the β-glucan chains. In contrast, other methods directly yield chitosan, chitin-glucan or chitosan-glucan products.

In a preferred embodiment, the chitin polymers contain more than 80% of chitin, and preferably more than 90% of chitin and even more preferred more than 95% chitin.

Furthermore, chitin obtained according to the present invention from fungal or yeast biomass, comprises lower crystalline index values than chitin polymers that are obtained from crustacean shells. In a preferred embodiment, the crystalline index of the chitin polymers is lower than 80%, and more preferably, below 70% and even more preferred below 65%, where chitin is obtained from an Aspergillus niger biomass. The crystalline index can be calculated by the method of Struszczyk et al. (J. Appl. Polym. Sci., 1987, 33:177-189).

In another embodiment, the invention relates to chitin-rich chitin-glucan copolymers obtainable by the method according to the present invention. In a preferred embodiment, said chitin-rich chitin-glucan copolymers have an adjustable amount of chitin, which is preferably higher than 80%.

In yet another embodiment, the invention relates to chitin-glucan copolymers obtainable according to the present method, in particular, before the enzymatic hydrolysis step. In a preferred embodiment where chitin is obtained from an Aspergillus niger biomass, said chitin-glucan copolymers obtained before the enzymatic hydrolysis step contain an amount of chitin preferably comprised between 30 and 50%.

Moreover, the present method does not induce degradation of the chitin chains, in contrast to other methods, which make use of concentrated alkali solutions. The present extraction method does not require the use of aggressive surfactants, nor acidic compounds. The method yields chitin from a renewable source, for example a fungi or yeast biomass, which is a valuable alternative source for crustacean shells. Moreover, the alkali solutions used in the method can be recycled in the course of the extraction process.

In another embodiment the invention relates to a method for isolating and purifying cell wall derivatives from a fungal biomass comprising the subsequent steps of suspending the biomass in an acidic solution and remove the acid soluble fractions, followed by suspending the acid-insoluble fraction in an alkaline solution and removing the alkali-soluble fraction, followed by further purifying and drying the alkali-insoluble fraction.

Advantageously this method is comprising the following subsequent steps:

1) Optionally suspending the biomass in an acidic solution and remove the acid soluble product,

2) Suspending the acid-insoluble fraction in an alkaline solution and removing the alkali-soluble product,

3) Purifying the alkali-insoluble product by further treatment with water,

4) Drying the water-insoluble product

5) Optionally purifying the dried product by treatment in organic solvent,

6) Optionally drying the organic-insoluble product.

The first step of suspending the biomass in an acidic solution is optional, depending on the initial biomass composition and on the purity required for the final chitin-glucan copolymer product. The acidic solution is preferably an aqueous solution of chlorhydric acid or sulfuric acid, and preferably chlorhydric acid. Preferably said acidic solution has a concentration comprised 0.1 and 5 N, and preferably of about 0.5N. This first extraction step allows eliminating acidic-soluble compounds, including inorganic compounds.

The second step makes use preferably of an aqueous solution of an alkali like sodium hydroxide, potassium hydroxide, ammonium hydroxide, and preferably sodium of potassium hydroxide. Preferably, said basic solution has a concentration comprised between 0.1 and 5 N, and preferably of about 0.5N.

The third step of purifying the alkali-insoluble product is preferably performed by contacting said product with water and separating the water-insoluble product for example by filtration.

Depending on the purity required for the final chitin-glucan copolymer product, a fifth step for further purification is used. It consists in suspending the dried product in an organic solvent like ethyl alcohol, ethyl acetate, acetone, more preferably ethyl alcohol. This step enables the removal of lipophilic compounds.

The first and second steps are preferably performed at a temperature preferably ranging between 5 and 120° C., and more preferably at a temperature lower than 60° C. The biomass is preferably ranging between 1 and 15% (dry weight, w/v), and more preferably 3 and 12%. These steps are each lasting preferably between 4 and 48 hours, and more preferably less than 30 hours.

Each step can be repeated several times, depending on the initial composition of the biomass and the purity required for the final chitin-glucan copolymer product.

Method for Repairing Chitosan

In another aspect, the present invention relates to methods for preparing chitosan. The present invention discloses the method for preparing chitosan having a higher molecular weight by a first process, and chitosan having a lower molecular weight by a second process.

In one process the invention relates to a method for preparing chitosan from chitin comprising the subsequent steps of
a) contacting said chitin with a basic solution, whereby an alkali-soluble fraction and an alkali-insoluble fraction is obtained and whereby said alkali-soluble fraction is discarded and said alkali-insoluble fraction comprising partially deacetylated chitin is retained,
b) contacting said alkali-insoluble fraction with an acidic solution, by suspending said alkali-insoluble fraction and bringing said suspended fraction into contact with said acidic solution in order to obtain a suspension of acidified alkali-insoluble fraction comprising said partially deacetylated chitin, and
c) contacting said acidified a suspension of alkali-insoluble fraction with a chitin deacetylase enzyme, whereby chitosan is obtained.

The chitin source for preparing chitosan in this method may comprise chitin of crustacean origin or chitin of fungal or yeast origin. In a preferred embodiment, the chitin source used is fungal chitin or yeast chitin obtainable by the above-described method according to this invention.

According to this method for preparing chitosan, chitin is treated in a concentrated solution of alkali so that the chitin chains are able to swell and that further access of chitin deacetylase to the chitin substrate is promoted. Preferred alkali solutions are sodium or potassium hydroxide solutions, used in amounts such that the weight ratio of alkali to chitin is ranging between 5 and 25, preferably between 10 and 25. To avoid chitin chains from degrading, and to promote the formation of a swollen chitin hydrogel, the alkali concentration is preferably as high as possible. In a preferred embodiment, said alkali solution comprises a concentration higher of 40% (w/v). The reaction takes place at a temperature of 50 to 120° C. In a preferred embodiment step a) is performed at a temperature comprised between 50 and 120° C., and more preferred between 80° C. and 120° C. In another preferred embodiment step a) is performed during a period comprised between 30 and 180 minutes, and preferably between 30 and 120 minutes. The alkali-insoluble fraction obtained in step a) is suspended and then diluted, filtrated and washed extensively with water.

Preferably, the alkaline solution used is collected after the first step, concentrated and recycled and re-used in the chitin isolation method of the present invention described above.

Then, the suspended alkali-insoluble fraction obtained is contacted with an acid solution, whereby an acidified fraction comprising partially deacetylated chitin is obtained. The pH of the suspension is adjusted to a value preferably below 7.0, and more preferably below 4.8, by addition of an acid, for example chlorhydric, acetic, formic, glutamic, phtalic acid, and preferably formic acid. This step takes place at room temperature.

Subsequently said acidified fraction obtained is contacted with a chitin deacetylase. Preferably a recombinant chitin deacetylase is used which is produced by a Pichia pastoris yeast that has been transformed with an expression vector carrying a DNA sequence encoding chitin deacetylase from Mucor rouxii. The recombinant chitin deacetylase (rCDA) to chitin ratio is preferably ranging between 0.5 and 10 mg/g chitin and more preferably between 0.5 and 5 mg/g. The deacetylase hydrolysis reaction is preferably performed at a temperature of 15 to 50° C., more preferably between 20 and 40° C., for duration of less than 120 hours, until the desired proportion of residual acetylated glucosamine units is reached.

It is important to note that this enzymatic step is performed under acid conditions. Preferably, the pH value during the enzymatic step is lower than 5.0, and even more preferred between 3.5 and 4.5. Unexpectedly, at this low pH values, good enzymatic deacetylation is obtained, although the optimal pH value of the recombinant deacetylase enzyme is comprised between 5.0 and 5.5. At the low pH values the enzyme remains active and the enzymatic deacetylation reaction can be advantageously performed within shorter times. Thus the CDA enzyme is used under reaction conditions which do not correspond to the optimal conditions for the stability and activity of the recombinant CDA enzyme. In fact, while the CDA enzyme is active under the optimal conditions of 60° C., and a pH preferably below 5.0, and more preferably comprised between 4.0 and 5.0, the present step is performed at different conditions, without being detrimental for the activity of the enzyme.

In a further embodiment, the method according to the invention comprises a further step which comprises precipitating said obtained chitosan. Herefore, the suspension is filtrated to eliminate non-deacetylated chitin chains, and the pH is adjusted to a value above 7.0 by addition of an alkali like sodium, potassium or ammonium hydroxide. The precipitated compound is then filtrated, washed, and either dried to yield chitosan in the amino form or resolubilized in acidic solution and freeze-dried. For example the precipitating compound can be solubilized in chlorhydric, acetic, citric, formic, lactic, glutamic, aspartic, glycolic, benzoic, sorbic (2,4-hexadienoic), oxalic, malic, tartric, ascorbic, lauric, or palmitic acid, or any other mineral or organic acid, or any other polyacid like for example hyaluronic acid or poly(acrylic acid).

This enzymatic method allows to recover higher molecular weight chitosan from chitin of fungal or crustacean origin, and also to control the final degree of acetylation at the desired value, by carefully choosing the conditions of the chitin deacetylase reaction, for example the pH or the duration of the reaction.

In another preferred embodiment of the present invention, the recombinant chitin deacetylase from Mucor rouxii expressed in Pichia pastoris (rCDA) can also be used to extend the deacetylation of chitosan, either from fungal or crustacean origin, with no loss of molecular weight.

For instance, a chitosan whose viscosimetric molecular weight is 500,000 Da and degree of acetylation is 19 mol % can be reacted with rCDA in a formic acid solution (1 N) at a polymer concentration of 0.5 g/l at pH 3.8 for 6 hours, at room temperature. The pH of the solution is then preferably increased over 7.0 by addition of an alkali like sodium, potassium or ammonium hydroxide to promote the precipitation of chitosan, which is preferably removed by filtration, subsequently washed and dried. In this example, the final degree of acetylation to comprised 10 mol %, and the molecular weight was not changed.

The enzymatic deacetylation method according to the invention for preparing chitosan advantageously allows producing highly deacetylated chitosan, with no loss of molecular weight and no loss of material, and no need for fractionation of the polymer chains. Since the method for producing the recombinant chitin deacetylase is a method intended for high volume fermentation batches, the amounts of chitin and chitosan that can be enzymatically transformed are suited for industrial production and use of the resulting highly deacetylated chitosan, in a very cost-effective and environmentally safe manner.

In a second process the invention relates to a method for preparing chitosan from fungal or yeast chitin comprising the subsequent steps of:
a) contacting said chitin with a basic solution, whereby an alkali-soluble fraction and an alkali-insoluble fraction is obtained and whereby said alkali-soluble fraction is discarded and said alkali-insoluble fraction is retained,
b) contacting said alkali-insoluble fraction with an acidic solution, by suspending said alkali-insoluble fraction and bringing said suspended fraction into contact with said acidic solution whereby an acid-insoluble fraction and an acid-soluble fraction is obtained and whereby said acid-insoluble fraction is discarded and said acid-soluble fraction comprising chitosan is retained.

In a preferred embodiment, the chitin source used is fungal chitin or yeast chitin obtainable by the above-described method according to this invention.

The method for preparing low molecular weight chitosan consists in a strong alkaline reaction at high temperature. An alkali like sodium, potassium, lithium, or ammonium hydroxide, and preferably sodium or potassium hydroxide, is added to the chitin suspension, such as the weight ratio of alkali to the dry chitin mass is preferably ranging between 1 and 20 (w/w), and more preferably between 1 and 15 (w/w). Additive can be used to minimize the degradation of chitin chains, for example sodium borohydride, thiophenol, and organic solvents such as methanol, ethanol, can also be added.

Preferably, the alkaline solution used is collected after the first step, concentrated and recycled and re-used in the chitin isolation method of the present invention described above.

Subsequently, the obtained alkali-insoluble fraction is separated and suspended. In a preferred embodiment said step is performed at a temperature higher then 80° C. Preferably, the suspension is placed at a temperature ranging between 80 and 140° C., more preferably between 100 and 120° C., and the reaction preferably takes place for a duration ranging between 30 and 300 minutes, more preferably less than 240 min.

The alkali-insoluble fraction is removed by filtration and washed with water. It is then solubilized in a diluted acidic solution, for instance chlorhydric, acetic, formic, and preferably acetic acid at a concentration of 0.1 to 1N. The acid-insoluble fraction is eliminated by filtration.

In a further embodiment, the method comprises a further step wherein chitosan from said acid-soluble fraction is precipitated by contacting said fraction with a basic solution. The pH of the acid-soluble fraction is preferably raised above pH 8.0 with an alkali solution like of concentrated solution of sodium or ammonium hydroxide. The precipitating compound is filtrated, washed repeatedly with water, and dried. The obtained compound is chitosan under the amino form. In an example, a chitosan with a degree of acetylation of 14 mol % and a viscosimetric molecular weight of 20 kDa (as determined by capillary viscosimetry) can be obtained.

In a further embodiment, also chitosan salts can be obtained from the acid-soluble fraction. Therefore, the acid-soluble fraction is precipitated by addition of an alkali solution like sodium or ammonium hydroxide. The precipitating compound is filtrated, washed repeatedly with water, and then solubilized in an acidic solution and then freeze-dried from this acidic solution. The precipitating compound can be solubilized in chlorhydric, acetic, citric formic, lactic, glutamic, aspartic, glycolic, benzoic, sorbic (2,4-hexadienoic), oxalic, malic, tartric, ascorbic, lauric, or palmitic acid, or any other mineral or organic acid, or any other polyacid like for example hyaluronic acid or poly(acrylic acid).

In another embodiment, the invention relates to chitosan polymers obtainable by the method according to the present invention.

In a preferred embodiment, the present invention relates to chitosan polymers having an adjustable molecular weight. Depending on the process and the conditions of the deacetylation reaction, chitosan having a low, medium or high molecular weight is obtainable. Preferably, said chitosan has a molecular weight comprised between 10 and 1000 kDA, as determined by Ubbelohde capillary viscosimetry.

In another preferred embodiment, the present invention relates to chitosan polymers having an adjustable degree of deacetylation. Depending on the process and the conditions of the deacetylation reaction, the acetylation degree can be tuned, in a range preferably comprised between 0 and 40 mol %.

### INDUSTRIAL APPLICATIONS

The present invention provides chitin polymers and chitin-rich chitin-glucan copolymers from non-animal origin obtainable by a method according to the present invention.

Chitin-glucan copolymers of the present invention, i.e. obtained before the enzymatic hydrolysis step according to the present method, comprise a portion of beta-glucan chains, the structure and composition of the copolymers being defined by the organism from which it is extracted. In a preferred embodiment, chitin-glucan copolymers are extracted from the mycelium of Aspergillus niger, and comprise mainly chitin and beta-(1,3)(1,4) and beta-(1,3) glucan chains. According to the invention, the amount of chitin and glucan in such polymers is further adjustable, depending on particular conditions applied during enzymatic hydrolysis, in order to obtain chitin-rich chitin-glucan copolymers.

Chitin polymers, (chitin-rich) chitin-glucan copolymers obtainable by a method according to the present invention provide interesting properties, which makes them suitable for being used in all kinds of applications. Major advantageous characteristics of these products include their wound healing properties and their chelating activity.

Also non-animal chitosan is obtainable by a method according to the present invention. Advantageously, starting from very pure chitin, very pure chitosan can be obtained. In addition, a controllable enzymatic deacetylation process enables to obtain chitosan of high molecular weight and at the same time an adjustable (low) degree of deacetylation. Also, due to the relative unlimited availability of fungal or yeast biomass large volumes of chitosan can be prepared, in a reproducible and adjustable way. Advantageously, such production is not subject to seasonal variation as it is the case when using a crustacean chitosan source.

Several problems are encountered when using chitosan from animal source in different applications. For instance, in nutritional application such chitosan is not suitable for vegetarians, can cause allergies to crustacean products, and requires the food products to be labelled accordingly. In cosmetic application such chitosan may cause allergy and there is a tendency for using non-animal products. Therefore, the non-animal chitosan obtainable by a method according to the present invention provides a solution for these issues.

Some of the interesting properties of the chitosan obtainable by a method according to the present invention include cationic charge, biodegradability, non-toxicity, chelating, wound healing, moisturizing. In addition the chitosan of the present invention does not induce allergic reactions and can provide an antifungal and antimicrobial activity.

Chitin and chitosan products obtainable according to the present invention may be used in multiple forms, depending on their application in various systems. Chitosan polymers may for instance be used in the form of an ammonium salt, as a diluted solution in different mineral and organic acids such as but not limited to chlorhydric, acetic, citric formic, lactic, glutamic, aspartic, glycolic, benzoic, sorbic (2,4-hexadienoic), oxalic, malic, tartric, ascorbic, lauric, or palmitic acid, or any other mineral or organic acid, any other polyacid like for example hyaluronic acid or poly(acrylic acid). The concentration of chitosan in such solution is preferably selected in function of the required viscosity. Therefore, according to the invention also solutions having different degrees of viscosity comprising chitin or chitosan products according to the present invention may be obtained.

Chitosan products obtainable according to the present invention can also be used in the form of a hydrogel. Such hydrogel may be prepared by using methods known in the art, for instance but not limited by preparation of a concentrated solution, by forming a complex with anionic (macro)molecules such as alginate, heparine, xanthan or pectin, by chemical crosslinking, or by forming covalent bonds between the amino-groups of the chitosan and other (macro)molecules. The products may also be used in the form of a thermo-reversible hydrogel.

Chitin and chitosan products obtainable according to the present invention may further be used in the form of a film. For instance, chitosan, prepared according to a method of the invention having a high molecular weight may have improved film-forming properties and can therefore provide more stable films. Also multi-layered membranes or substrates comprising chitosan in association with other polymers can be prepared.

Moreover, chitin and chitosan products obtainable according to the present invention can further be used to manufacture as porous films or porous object, from which the pore sizes are controllable by applying methods known by a person skilled in the art.

In another embodiment, chitin and chitosan products obtainable according to the present invention can be provided in the form of micro-, milli- or nano-particles, which can be obtained by techniques known by a person skilled in the art (e.g. see Polymeric Biomaterials, S Dimitriu E D, Marcel Dekker, 2002, Chap. 1). Chitosan products obtainable according to the present invention and provided in the form of particles can have multiple application possibilities including encapsulation of substances, organisms or active molecules such as seeds, cells, pigments, flavours, odorous substances, drugs, vaccines, bioactive (antibacterial or antifungal) agents, enzymes. The encapsulation in chitosan particles makes it possible to immobilize, protect, transport, or to release the active substances in a controlled way.

Chitin-glucan copolymers of the present invention are essentially not soluble in any solvent, although they are hydrophilic, and are therefore suitable for being used in the form of powder, fibers or in a lyophilized form.

In another embodiment of the present invention, composite material is provided comprising chitin polymers, (chitin-rich) chitin-glucan copolymers or chitosan polymers obtainable by a method according to the present invention. Chitin polymers or chitosan polymers of fungal origin according to the present invention can be used in a mixture with one or more other substances. It can for example be mixed with other polymers, the mixture being usable in one of the forms as mentioned above, in order to confer new properties or synergetic properties.

Chitin polymers, (chitin-rich) chitin-glucan copolymers or chitosan polymers obtainable by a method according to the present invention can be mixed with molecules of low molecular mass. In combination with other substances, chitin polymers, (chitin-rich) chitin-glucan copolymers or chitosan polymers obtainable by a method according to the present invention are also suitable as complexing agents, if the substance presents a negative charge, or suitable as matrix for the controlled release of a drug or an active agent or suitable as matrix for a cosmetic ingredient such as a pigment, a flavour, or an odorous substance.

Chitosan polymers obtainable by a method according to the present invention can also be mixed with a vaccine, wherein they are suitable as adjuvant. Chitin polymers, (chitin-rich) chitin-glucan copolymers or chitosan polymers can further also be mixed with an inorganic substances, for instance with ceramics, preferably calcium phosphates, whereby a matrix can be created which is suitable for supporting tissue regeneration such as a cartilage or bones.

Another embodiment of the present invention relates to derivatives of chitin polymers, (chitin-rich) chitin-glucan copolymers or chitosan polymers obtainable by a method according to the present invention. Chitin polymers, (chitin-rich) chitin-glucan copolymers or chitosan polymers are polymers that can be modified chemically to obtain derivatives, according to techniques known by a person skilled in the art. The chemical modification can for instance be carried out on one or more functional groups of the D-glucose, N-acetyl-D-glucosamine or D-glucosamine units, for example on the oxygen atom in position 6, or on the nitrogen atom in alpha of the carbon located in position 1 in the N-acetyl-D-glucosamine and D-glucosamine.

Chitin polymers, (chitin-rich) chitin-glucan copolymers or chitosan polymers obtainable by the methods according to the invention may be applied in various products and systems, preferably as in medical, pharmaceutical, agricultural, nutraceutical, food, textile, cosmetic, industrial and/or environmental applications.

In a preferred embodiment chitosan polymers according to the present invention may be used as excipient in the preparation of a medicament. They may be used in veterinary as well as human medical applications. The invention also relates to a pharmaceutical composition comprising chitosan polymers according to the present invention. To enable the use of chitosan in pharmaceutical forms, controlled and reproducible molecular weight distribution, degree of acetylation, and low and reproducible levels of impurities of the compounds are required. According to the methods of the present invention, compounds with such characteristics can be obtained.

Chitin polymers, (chitin-rich) chitin-glucan copolymers or chitosan polymers are not antigenic and are perfectly biocompatible. Moreover, they are biodegradable by enzymatic hydrolysis, for example in the presence of lyzozymes. Due to their anti-thrombogenic and haemostatic character they can be used in all fields of medicine. Therefore, chitin polymers, (chitin-rich) chitin-glucan copolymers or chitosan polymers obtainable by the methods according to the invention may be applied in wound healing systems. Chitin polymers, (chitin-rich) chitin-glucan copolymers or chitosan polymers obtainable by the methods according to the invention may also be used to prevent the formation of fibrin bits in wounds, and to prevent the formation of scars, and to support cell regeneration. Chitin polymers, (chitin-rich) chitin-glucan copolymers or chitosan polymers may be used in systems for tissue engineering, cell transplantation and cell encapsulation. Since the products may form air-permeable films, they can support cellular regeneration while protecting tissues from microbial aggressions. They may also be used to form sutures, bandages, and preferably to form degradable sutures and bandages. Chitosan polymers obtainable by the methods according to the invention are further suitable for manufacturing artificial skin and in systems for reconstruction of tissues and organs and/or the transplantation of cells. For example, chitin polymers, (chitin-rich) chitin-glucan copolymers or chitosan polymers may be used in systems for osseous repair in orthopaedics or orthodontics, for repair of the skin, the cornea, the retina, the cartilage or for the reconstruction of organs as pancreas, stomach, and nervous systems.

Chitosan polymers according to the present invention are also suitable for use in contact lens, dry eye prevention compositions, as a tear substitute in the form of a topical hydrogel, as a topical carrier for ocular drugs, as a particulate or hydrogel systems for local delivery inside the eye, in devices to repair retinal detachment and macular degeneration and in surgical aids for surgery.

Due to good bio-adhesion properties, chitosan polymers according to the present invention can be applied as anti-adhesive surgical aid, for instance to prevent adhesion between tissues during surgery. They can also be applied as adjuvant for vaccines thanks to a good mucoadhesion.

Chitosan polymers obtainable according to the present invention can be further applied as support for transport and slow-release of active compounds in plants, animals and human. With regard to the oral administration forms of pharmaceuticals, it is particularly suitable to use chitosan polymers when encapsulated products must arrive without transformation in the intestine, since the products are not digested by the stomach. Chitosan can be formulated as particles, which gives even more opportunities for oral and parenteral controlled release applications. Chitosan can increase the efficacy of oral carriers by chemical modification and binding of drugs or other bio-functional molecules. Because chitosan polymers possess good film and gel forming properties, it can serve to manufacture transdermal membranes. Its muco-adhesive properties are desired for a good contact with the outer skin layer. Chitosan can also be useful to prepare innovative drug delivery systems for local and systemic routes of administration, like the vaginal, buccal, and parenteral routes.

Chitin polymers, (chitin-rich) chitin-glucan copolymers or chitosan polymers obtainable according to the present invention can be used as an excipient in the formation of tablets, the granulation of powders, the making of gels and films, the preparation of emulsions, and also as a wetting and coating agent. Some more original properties of chitosan can also be exploited in oral drug delivery systems, like its ability to provide a drug controlled release as a matrix, its bioadhesiveness, its film-forming properties, its ability to form complexes with anionic drugs and anionic polymers. Therefore, they may be used to in drug systems to improve the solubility of poorly water soluble drugs, to form hydrogels to enhance absorption of drugs across mucosal tissues, to potentiate immunological response of vaccines.

In another embodiment, chitin polymers, (chitin-rich) chitin-glucan copolymers or chitosan polymers obtainable according to the present invention can be further applied in agricultural and agrochemical systems. They may be applied as preservative coating and biofungicide when applied on fresh fruits, vegetables and crops, or as fertilizers, thereby increasing the number of useful soil microorganisms and decreasing harmful ones. Plant seeds may be soaked in aqueous solutions of chitosan to prevent microbial infections and increase plant production. Chitin polymers, (chitin-rich) chitin-glucan copolymers or chitosan polymers according to the present invention can further be used in solution, powder or coating of seeds. In low amounts, about a few milligrams per cubic meter of water, chitin polymers, (chitin-rich) chitin-glucan copolymers or chitosan polymers can be used to trigger plant defense mechanisms against parasitic infections and aggressions. In addition to anti-fungal properties, chitin polymers, (chitin-rich) chitin-glucan copolymers or chitosan polymers can be applied to reinforce the plants' root and to thicken the plants' stem. Chitin polymers, (chitin-rich) chitin-glucan copolymers or chitosan polymers according to the present invention can also be used to stimulate the synthesis of protective agents by a plant. Furthermore, they can be used to accelerate the germination and the growth of plants. In the agro-alimentary sector, they can be used for the coating of seeds, manure or pesticides.

Due to their film-forming properties, chitosan polymers of the invention may be used as additives in pesticides for providing a better contact and a better penetration of the pesticide. Furthermore, association of the pesticide with a small quantity of chitin or chitosan of the present invention may be suitable to decrease the amount of pesticide used.

In another embodiment, chitin polymers, (chitin-rich) chitin-glucan copolymers or chitosan polymers obtainable according to the present invention are further particularly suitable for use in nutraceutical and food applications. Chitin polymers, (chitin-rich) chitin-glucan copolymers or may be used as food supplements. In particular, chitosan polymers may be applied as food ingredient in dietetics. As chitosan polymers are not digested by the human body, they are suitable for behaving like a fibre, which is a significant element in a diet. As chitosan polymers bear cationic charges, they are able to complex negatively charged lipids and they are suitable for trapping lipids in the digestive tract. In addition, chitosan polymers may be applied in nutraceutical products for obtaining hypo-cholesterolemic effects

In addition, chitosan polymers according to the present invention can be used as natural food additives for obtaining anti-microbial and anti-fungal activity against a wide range of food-borne fungi, yeast and bacteria. In addition, they may be used as adjuvant for conventional food preservatives, as anti-browning agents, as component for gas permeable edible films suitable for fruit/vegetable storage, as thickening, stabilizing or emulsifying agent, as thixotropic agents or as natural flavour extender. In addition, chitosan polymers according to the present invention may be used in food processes, where they may for instance be applied as foaming agents, as thickener or stabilizer. Due to their coagulant and flocculating capacities, chitosan polymers may also be applied in the clarification process of beverages like wine, beer and fruit juices. Herein they may precipitate compounds responsible for the haze of these beverages.

In another aspect of the food industry, chitosan polymers can also be used to prepare edible films and coatings to extend shelf life of fresh or processed food. Fungal chitosan polymers can be applied directly on fruits and vegetables, which allows extending shelf life, a better control of fruits/vegetable decay and delaying of ripening. Chitosan polymers are suitable as anti-browning agent on fruits and vegetables. They can then be used as an advantageous alternative to sulfite, the most effective browning inhibitor currently available although suspected to provoke adverse health effects.

In another aspect, the anti-microbial and anti-fungal activities of chitosan polymers according to the invention can be exploited in the food industry, for the preservation of meat, crustacean (oysters), fruits, vegetables and finished products, either alone or in synergetic combination with conventional preservatives like for example sulphite or sodium benzoate. When associated with other preservatives, it may be used to minimize the preservative concentration necessary for an inhibition effect.

In another embodiment, chitin polymers, (chitin-rich) chitin-glucan copolymers or chitosan polymers obtainable according to the present invention are further usable in textile applications. Chitosan polymers can for instance be applied on textile fibers in the form of a film by impregnating said fibers or a tissue with a solution. By doing so, the properties of the fibers or textiles may be changed, e.g. by application of chitin or chitosan such fibers or textiles may adopt an anti-bacterial character. Medical textiles can also be impregnated by chitin polymers, (chitin-rich) chitin-glucan copolymers or chitosan polymers according to the present invention and be suitable in systems for the treatment of wounds.

In cosmetic applications, chitin polymers, (chitin-rich) chitin-glucan copolymers or chitosan polymers obtainable according to the present invention are usable is compositions suitable for care of skin, such as creams, and for the hair, such as sprays, shampoos and after-shampoos, in make-up compositions, or in tooth pastes. They are further applicable in anti-UV compositions, in the preparation of deodorants, in compositions for oral hygiene and in compositions for encapsulation of pigments. The non-animal origin of the chitin or chitosan obtained according to the method described in the invention makes it possible to eliminate risks of allergies.

In environmental applications, chitin polymers, (chitin-rich) chitin-glucan copolymers or chitosan polymers obtainable according to the present invention may be applied as chelating agents, e.g. as heavy metal complexing agents. Chitin polymers, (chitin-rich) chitin-glucan copolymers or chitosan polymers may be applied for trapping heavy metals and in water purification techniques, or they can be applied in drinking water system for separating organic compounds and heavy metals. They can also be applied for treating water by precipitating certain waste and by capturing pollutants like DDT and polychlorobenzenes. In addition, they may also be used in applications wherein they are suitable for fixing radicals.

Moreover, chitin polymers, (chitin-rich) chitin-glucan copolymers or chitosan polymers according to the present invention may be used in the manufacturing process of paper. In this process they may replace some amino substituents such as gum or polysynthetic polysaccharides and they are suitable for reducing the use of chemical additives and to provide improved outputs. Paper produced by using chitin polymers, (chitin-rich) chitin-glucan copolymers or chitosan polymers according to the present invention may have a smoother surface and show better resistance to moisture. Moreover, chitin polymers, (chitin-rich) chitin-glucan copolymers or chitosan polymers according to the present invention may also be applied for production of sanitary paper, packing paper and paperboard.

One particular aspect of the invention is a method for treating food-grade liquids. There existed in the technical field the preconception that a technological additive for treating food-grade liquids, especially food-grade liquids of plant origin possibly obtained by alcoholic fermentation, preferably wines, beers, champagnes, ciders and fruit juices, should be advantageously charged, especially positively charged.

Thus, to solve the technical problems listed above, a person skilled in the art who would have had the idea of using a natural polymer of non-animal origin might have used chitosan of plant origin to perform the treatment of food-grade liquids, since chitosan is a cationic polymer and can therefore capture anionically charged undesirable compounds. Specifically, the present inventors have already invented a process for producing chitosan of non-animal origin, from plant sources, more particularly of fungal origin and more particularly from a fungus of Aspergillus niger type. However, the inventors have discovered, surprisingly, that chitosan is not the best indicated polymer accessible via the process described above and in international patent application WO 03/068 824 for clarifying and/or stabilizing food-grade liquids.

Surprisingly, the present inventors have discovered that an extract of fungal biomass predominantly comprising at least one nonionic polysaccharide may be used very satisfactorily in the treatment of food-grade liquids, especially food-grade liquids of plant origin, possibly obtained by alcoholic fermentation, and preferably wines, beers, champagnes, ciders and fruit juices. This nonionic polysaccharide is preferably a chitin-glucan copolymer or a hydrolysate thereof.

Thus, the present invention relates to the use of an extract of fungal biomass predominantly comprising at least one nonionic polysaccharide, as a technological additive for treating food-grade liquids, especially food-grade liquids of plant origin, possibly obtained by alcoholic fermentation, and preferably wines.

By the term “technological additive” the inventors mean any substance not consumed as a food ingredient per se or deliberately used in the transformation of raw materials and possibly having as a result the unintentional presence of technically inevitable residues of this substance or of derivatives thereof in the finished product. Technological additives especially do not form part of the ingredients of the food-grade liquid: they are used only during the preparation of the product to facilitate it, but are not included in the composition of the finished product.

By the term “treatment of food-grade liquids”, preferably wines, the inventors especially mean any operation for stabilizing the liquid by removing the compounds responsible for cloudiness or instability over time, for making the liquid fit for consumption, especially by improving its appearance or taste, while at the same time bringing the impurity levels below levels defined by the legislation in force.

Examples of “food-grade liquids of plant origin” are fruit juices, and examples of “food-grade liquids of plant origin obtained by alcoholic fermentation” are fermented beverages (wines, beer, etc.) and spirits (whisky, brandy, etc.). The food-grade liquids of plant origin that may be treated with the fungal extract of the present invention are not limited and are chosen, for example, from alcoholic beverages (wines, ciders, champagnes, etc.), liquers (liquer wines, port, fruit liquers, etc.), distilled beverages (cognac, gin, tequila, brandy, etc.), alcoholic beverages (pastis, cocktails, etc.), fruit juices (including vegetables), soups, vinegars, including mixtures thereof, and a mixture of one of the abovementioned beverages of plant origin with another beverage of non-plant origin to prepare a food-grade liquid mixture, for instance a mixture of milk and of fruit juice. Advantageously, the food-grade liquid of plant origin is chosen from a fermented beverage and a fruit juice.

By the expression “predominantly at least one nonionic polysaccharide” the inventors mean an extract comprising an effective amount of nonionic polysaccharide to be used as technological additive according to the present invention, present in the technological additive in an amount greater than that of the other compounds present. The amount of nonionic polysaccharide to be used in the technological additive may be determined by a person skilled in the art, and is preferably greater than 70% by mass relative to the total mass of the total technological additive, preferably greater than 75%, preferably greater than 80%, preferably greater than 85%, preferably greater than 90% and more preferably greater than 95%. The other compounds present in the technological additive do not act in the phenomenon of treatment of the food-grade liquid, and it is therefore preferable to partially or totally remove them, which increases the capacity to treat the food-grade liquid at an equivalent dose.

The additives behave especially like a layer of filtering material and are therefore not present in the final liquid.

In general, the present invention using fungal extracts is very simple to use. The fungal extract according to the present invention is preferably used in the form of a powder that flocculates by adsorbing the undesirable compounds. Its use is compatible with the practices used for treating food-grade liquids that are commonly used at the present time, does not require any special equipment, and is compatible with the usual price of the treatments used, in particular for oenological treatments. They are therefore accessible to all producers.

Thus, it is possible, for example, to use the fungal extracts according to the present invention at a concentration of between 1 g/hl and 1 kg/hl of liquid to be treated. Preferably, an amount of between 10 g and 500 g per hl of liquid to be treated and more preferably between 10 and 200 g/hl of liquid to be treated is used.

It is possible, for example, to add the fungal extract according to the present invention to the liquid to be treated contained in a tank, which is advantageously stirred to mix in the fungal extract. This operation may be performed at room temperature (20-25° C.), but may also be performed under hot or cold conditions within reasonable limits so as not to deteriorate the future beverage. This operation may be performed for a period of between a few hours and a few days, which is preferably adjusted by a person skilled in the art. Next, the fungal extract is advantageously separated form the liquid via methods known to those skilled in the art, for instance filtration or decantation.

The capacity for production of these fungal extracts, associated with the presence of a renewable fungal source, gives access to volumes that are compatible with the needs of the food industry, for instance for the production of wines, beers, champagnes, ciders or fruit juices.

The fungal extract according to the present invention may be used in any step of the treatment of the food-grade liquid, and preferably in a maximum number of steps of this treatment.

Advantageously, the fungal extract according to the present invention allows the partial or total removal of undesirable compounds, which are the causes of instability or of health risks.

Advantageously, the undesirable compounds are chosen from the group consisting of colloids causing instability, colloids causing cloudiness, colloids that produce poor-quality organoleptic properties, proteins, metals, heavy metals, in particular copper, iron, cadmium and lead, residual pesticides, for instance fungicides, insecticides and herbicides, and toxins, for instance mycotoxins and bacterial endotoxins, and that their removal has the aim of improving the quality of the food-grade liquid.

Advantageously, the fungal extract according to the present invention allows the treatment of finished food-grade liquids (treatments after fermentation, for instance bottling or élevage).

Thus, undesirable compounds in the food-grade liquid can be removed to obtain a ready-to-drink beverage, or the composition of the liquid can be modified to obtain a beverage whose preferred composition (color, taste, etc.) is optimized.

The advantage of using the fungal extracts proposed by the inventors is that they make it possible to obtain efficacy in particular in the treatment of musts, wines and alcoholic beverages, to avoid iron breakage, to remove oxidation products, to remove possible biocides (pesticides, herbicides, fungicides, etc.) and/or proteins without substantially touching the other constituents of the food-grade liquid, for instance the phenolic compounds especially for wine, and while at the same time avoiding any risk of leaching of residues, and any risk of allergenicity.

Another advantage of the fungal extracts is that they allow a reduction of the toxic contaminants of various beverages, for example musts, wines and spirits, etc., such as mycotoxins, heavy metals (lead, cadmium), major metals (iron) and pesticides.

In the context of the present invention, the technological additive is advantageously used for the treatment of beverages of plant origin, for instance fruit juices, wines and/or other beverages derived from fermentation, for instance beer, champagne or cider, and makes it possible especially:
a) to remove soil particles;
b) to remove organic particles in order to reduce the phenol oxidase activity;
c) to reduce the indigenous microbial flora;
d) to reduce the colloid content and the turbidiy;
e) optionally to reduce the presence of polyphenol compounds of the must in order to lower its astringency, before fermentation;
f) to remove the insoluble particles in the must;
g) to facilitate the stripping of new wines via the partial precipitation of the excess protein matter;
h) to perform a preventive treatment for protein and copper breakage;
i) to correct the organoleptic properties of wines derived from musts impaired by fungi, for instance rot or oidium;
j) to remove possible contaminants;
k) to correct the color:
   of white musts obtained from red grapes giving white juice (possibly stained),
   of very yellow musts obtained from white grape varieties,
   of oxidized musts;
l) to reduce the indigenous population of microorganisms before the alcoholic fermentation for the subsequent inoculation of the selected yeasts;
m) to precipitate the particles in suspension: either by promoting the free fall of these particles, or by coagulating around the particles to be removed, entraining them into the sediments;
n) to soften red wines by removing some of their tannins and polyphenols;
o) to clarify wines that are cloudy because of breakage, rising of the lees, insolubilization of coloring matter, etc.;
p) to obtain clarity for the wine;
q) to obtain biological stability for the wine by removal of microorganisms (sterilizing filtration);
r) to facilitate the stripping of new wines via the partial precipitation of the excess protein matter;
s) to remove an excess of colloidal copper used during the treatment of wine with copper sulfate pentahydrate to remove the poor taste and odor caused by hydrogen sulfide and possibly derivatives thereof;
t) to remove the excess iron from the wine, preventing iron breakage by use with combined oxygenation;
u) to prevent protein and copper breakage: to protect the wine against mild iron breakage, to prevent the precipitation of substances such as coloring matter which, in wine, are in colloidal form;
v) to fix the ferric ions and thus to reduce the tendency to iron breakage;
w) to reduce the iron content of the wine in order to prevent iron breakage, or the copper content in order to prevent copper breakage, and more generally to reduce the content of heavy metals;
x) to prevent iron breakage in the case of iron-rich wines not containing an excess of copper; or
y) to reduce the content of tannins and other polyphenols of the wine in order to combat the tendency towards browning, to reduce the astringency or to correct the color of stained white wines.

The technological additive according to the present invention is not an enzymatic preparation to be added to the must or wine in order to improve the filterability by enzymatic hydrolysis, especially by enzymatic hydrolysis of the pectins and/or glucans given to the must or wine by Botrytis cinerea and/or certain yeast strains.

According to one advantageous embodiment, the technological additive according to the present invention is used for treating a fermented alcohol, which is a liquid that is particularly difficult to treat while conserving its organoleptic properties.

According to one particular embodiment, the present invention relates to the use of an extract of fungal biomass for the clarification of a food-grade liquid of plant origin and preferably for the clarification of wine.

By the term “clarification of wine” the inventors mean the separation, before or during fermentation, of the more or less clear liquid from the solid matter in suspension in the must and/or wine using suitable additives.

The additives used should comply with the legislations in force, and in the case of wine they should comply with the prescriptions of the Codex Oenologique International. The technological additives according to the present invention are advantageously extracts of fungal biomasses predominantly comprising nonionic polysaccharides, advantageously predominantly comprising at least one chitin-glucan copolymer.

Advantageously, the nonionic polysaccharide predominantly comprises N-acetyl-D-glucosamine (chitin) and D-glucose (beta-glucan) units.

Preferably, the chitin-glucan copolymer is a copolymer that predominantly comprises macromolecular chains of N-acetyl-D-glucosamine units linked together preferably via alpha(1,6) bonds (commonly known as chitin) and macromolecular chains of D-glucose units linked together preferably via beta bonds (beta-glucan), for example of beta(1,3), beta(1,4), beta(1,3-1,4) or beta(1,6) type, preferably with a chitin/glucan ratio ranging from 95:5 to 5:95, preferably from 70:30 to 20:80 and more preferably from 70:30 to 30:70 (m/m).

Another preferred chitin/glucan ratio is comprised between 50:50 and 10:90, or between 45:55 and 20:80. This ratio may be obtained by hydrolyzing chitin-glucan copolymers described above. These extracts are totally insoluble in food-grade liquids such as wine, beer, fruit juices, etc.

Advantageously, the chitin-glucan copolymer is an (N-acetyl-D-glucosamine)-(D-glucose) copolymer.

Advantageously, the technological additives according to the present invention are obtained via the process described above or in international patent application WO 03/068 824 filed in the name of KitoZyme S. A. on Feb. 12, 2003, which is incorporated herein entirely by reference.

In one preferred embodiment, the invention relates to a method wherein the biomass is chosen from the group consisting of filamentous fungi such as Aspergillium, Penicillium, Trichoderma, Saccharomyces, and Schizosaccharomyces, and edible fungi such as Agaricus, Pleurotus, Boletus, and Lentinula, and/or a mixture thereof. The invention includes genetically modified organism (GMO). The common characteristic of these fungi is the presence of polysaccharides in their cell wall, preferentially chitin and/or beta-glucan. According to one preferred embodiment, the fungal extracts are obtained from Aspergillus niger or shiitake, in a very economically viable manner. The fungal extract is preferably Ascomycetes, in particular Aspergillii.

The process according to the present invention comprises the placing in contact of the biomass with a basic solution, in which a fraction that is soluble in alkaline medium and a fraction that is insoluble in alkaline medium are obtained and in which the fraction soluble in alkaline medium is discarded and the fraction insoluble in alkaline medium, which predominantly comprises nonionic polysaccharides, is retained.

The fraction insoluble in alkaline medium predominantly comprises nonionic polysaccharides that advantageously comprise at least one chitin-glucan copolymer.

Advantageously, the concentration of the alkaline reagent is preferably between 0.1% and 40% (m/v), preferably less than 10% and preferably less than 1%. The reaction is performed at a temperature preferably ranging from 5 to 120° C., preferably less than 60° C. and more preferably at room temperature. In one embodiment, the reaction is preferably performed for less than 4 hours. The first extraction step makes it possible to remove the compounds that are soluble in alkaline medium, including the pigments, the proteins, some lipids and some polysaccharides other than the nonionic polysaccharides. Advantageously, at least some of the lipophilic compounds whose residues may be released into the food-grade liquid, resulting in impairment of its taste, are removed.

According to one preferred embodiment, the biomass may be treated with the first alkaline solution, filtered via a technique known to those skilled in the art, preferentially using a filter press, and treated again with a second alkaline solution at an alkaline reagent concentration equivalent to that of the first step or different. Additional additives may be used to improve the extraction of the polysaccharides desired for the present invention. Such additives are preferably chosen from organic solvents, for example cyclohexane, ethyl acetate, methanol or ethanol; antifoams such as structol; surfactants such as sodium dodecyl sulfate, poly(vinyl alcohol), Tween or a poloxamer; enzymatic preparations containing carboxylesterase, carboxylic ester hydrolase, or triacylglycerol lipase, and bleaching agents, for instance hydrogen peroxide. A step prior to the alkaline treatment may consist of one or two rapid treatments in acidic medium, using a mineral acid.

To isolate the product insoluble in alkaline medium from the cell biomass that predominantly comprises nonionic polysaccharides of chitin-glucan type, the first step is followed by repeated washing with water, followed by filtration, a treatment for removing the lipid compounds using an organic solvent, for instance ethanol, filtration and drying. Preferably, the filtration is performed with a filter press.

To enrich with chitin the extract obtained, which initially predominantly comprised chitin and beta-glucan polysaccharides in the form of a copolymer, the first step is followed, for example, by repeated washing with water, followed by other steps as described below. A chitin-rich copolymer may thus be obtained.

A second step of the process according to the present invention comprises placing in contact the fraction insoluble in alkaline medium with an acidic solution, suspending said fraction insoluble in alkaline medium and bringing said suspended fraction into contact with the acidic solution so as to obtain an acidified suspension of the fraction insoluble in alkaline medium comprising the nonionic polysaccharides.

After the last filtration step described above, the product insoluble in alkaline medium may be suspended in water so as to obtain a concentration preferably of between 1% and 8% (m/v) and preferably between 1% and 5% of insoluble product suspended in water. Next, the pH of the aqueous suspension of product insoluble in alkaline medium is adjusted to below 7.0 by adding an acidic solution, preferably below 6.0 and preferably greater than 3.0.

The acidic solution is preferably an aqueous solution of an acid, for instance hydrochloric acid, acetic acid, formic acid, lactic acid, glutamic acid, aspartic acid or glycolic acid, and preferably acetic acid. This step is preferably performed at a temperature of between 5 and 60° C. and preferably below 30° C.

A third step of the process according to the present invention comprises the placing in contact the acidified fraction insoluble in alkaline medium with at least one enzymatic preparation that is rich in enzymes of beta-glucanase activity, the beta-glucanase enzyme making it possible to obtain the extract of fungal biomass that predominantly comprises chitin-enriched nonionic polysaccharides. Advantageously, the method is wherein the enzymatic preparations contain at least one enzyme of beta-glucanase activity chosen from the group of endo-beta-(1,3)-glucanase, exo-beta-(1,3)-glucanase, beta-(1,3) (1,4)-glucanase or beta-(1,6)-glucanase activity, and any mixture thereof.

Advantageously, a mixture of enzymes is added to the acidified suspension of the fraction insoluble in alkaline medium to hydrolyze the beta-glucan chains associated with chitin. The hydrolysis reaction is preferably performed at a temperature of between 5 and 60° C. and more preferably below 40° C. The reaction time is preferably less than 5 days. Preferred preparations are illustrated in below examples and in patent application WO 03/068 824.

Advantageously, the extract of fungal biomass comprises at least one chitin-glucan copolymer, which is advantageously enriched in chitin. The chitin/glucan ratio may be readily adjusted by controlling the reaction conditions, especially by means of the beta-glucanase used and by the reaction time.

It is possible, for example, to obtain a chitin-glucan copolymer comprising an amount of chitin (poly(N-acetyl-D-glucosamine)) of less than 60% by mass relative to the total mass of the copolymer, preferably less than 50% and more preferably between 20% and 50%. This copolymer is especially obtained after the first treatment step with an alkaline solution. Examples for obtaining this copolymer are given below and in patent application WO 03/068 824, examples 1 and 2.

It is also possible, for example, to obtain a chitin-glucan copolymer comprising an amount of glucan (poly(D-glucose)) of less than 30% by mass relative to the total mass of the copolymer, and preferably less than 25%. This chitin-enriched polymer is obtained after the third step of treatment with an enzyme with beta-glucanase activity. Examples for obtaining this copolymer are given in below (examples 4 and 5).

In another particular aspect, the present invention describes an application of the extract, advantageously purified, of a fungal source, and preferably of Aspergillus niger. The hydrolysates of the purified extracts, i.e. the copolymers of chitin and of beta-glucan of lower molecular mass, are also part of the invention. It is the association between the two polymers (chitin and beta-glucan) and the three-dimensional architecture of the copolymer derived form the fungal source which makes it a compound beneficial to the health. The availability and the quality in particular of Aspergillus niger, which is a coproduct of the production of citric acid intended for the food and pharmaceutical industry, make it a starting material of choice for uses of its derivatives, in human and animal health.

Fibre of polysaccharide type and oligomers thereof of various origins are also known for their beneficial actions, such as oligofructoses, laminarin or chitosan. The present inventors had already described a process for producing chitosan of nonanimal origin, from fungal sources, and more particularly from a fungus of Aspergillus niger type, in international application WO 03/068824.

Now, the inventors have discovered, surprisingly that the chitin-glucan compound derived from the fungal sources mentioned above, and in particular from the fungus Aspergillus niger, and its hydrolysates, surprisingly combine several of the known properties of fibre, and that the chitosan is not the compound most indicated for overcoming dyslipidemias, for example. In fact, the chitin-glucan compound and its hydrolysates exert both blood-cholesterol-lowering and hypoglycaemic effects, they increase the antioxidant capacity of plasma, thus promoting a decrease in atheroma plaque, they promote satiety and stimulate the immune system.

As regards chitosan, it seems to be the case, a priori, that its slimming action is exerted by virtue of its ability to bind lipids at the gastric level, and that its blood-cholesterol-lowering action is exerted by virtue of its ability to bind lipids at the intestinal level, via electrostatic interactions between fatty acids or bile acids and the ammonium groups of chitosan. Now, the chitin-glucan substances and hydrolysates thereof of the invention, firstly, essentially exert no electrostatic charge and, secondly, are not capable of taking up fatty acids in vitro. Nevertheless, they inhibit a significant reducing action on triglyceride content, on total cholesterol content and on LDL-cholesterol, which is equivalent to that of chitosan, while at the same time promoting an increase in HDL-cholesterol in a manner equivalent to that of chitosan.

Added to these common actions are effects that are not described with chitosan, which make them advantageous substances: chitin-glucan and its hydrolysates are capable of absorbing generally approximately 10 times their mass in water, which makes them substances with good fibre properties capable in particular of improving transit. They are capable of significantly stimulating plasma antioxidant activity and of exercising an immunostimulant activity due to the synergy between the chitin part and the beta-glucan part.

Since they are not capable of taking up lipids at the gastric level, the chitin-glucan and its hydrolysates do not bring about risks of lipophilic nutrient (vitamin) imbalance, as is sometimes suspected for chitosan. Finally, the chitin-glucan and its hydrolysates are obtained and purified according to a process that is readily carried out, starting from fungal sources of excellent quality, which are coproducts that are available in large amount and renewable, and which are not of animal origin.

The inventors mean, by “polysaccharides of fungal origin”, the extracts purified from fungal cell walls composed predominantly of polysaccharides of chitin and of beta-glucan, in the form of copolymers, and hydrolysates thereof. The purified extracts preferably comprise a chitin-glucan content of greater than 70% by mass relative to the total mass of the extract, preferably greater than 80%, preferably greater than 85%, and more preferably greater than 95%.

The inventors mean, by “chitin-glucan”, a pure copolymer extracted from fungal cell walls which consist of links of N-acetyl-D-glucosamine units and, optionally, of a minor proportion of D-glucosamine units linked to one another by (1,6)-type linkages in the alpha conformation (chitin link), and of links of D-glucose units linked to one another by (1,3)-type, (1,3)(1,6)-type or (1,3)(1,4)-type linkages, and preferably (1,3)-type linkages, in the beta conformation (beta-glucan link, also referred to herein as “glucan”).

It is generally accepted that fungal cell wall polysaccharides can be separated into two groups according to their solubility in an alkali medium, and that the cell wall backbone is insoluble. It is also known that the insoluble fraction consists of chitin and of beta-glucans, in variable proportions according to the species, that the beta-glucan units are linked by links of variable structure, and that the linkage between the chitin and beta-glucans is stable, as shown, for example, by Siestma & Wessels for Saccharomyces cerevisiae (Zygomycete), Neurospora crassa (Ascomycete), Aspergillus nidulans (Ascomycete) and Coprinus cinereus (Basidiomycete) (Siestma J H & Wessels J G. (1981) Solubility of (1,3)-beta-D-(1,6)-beta-D-glucan in fungal walls: importance of presumed linkage between glucan and chitin. (1981) J. Gen. Microbiol. 125:209). It is known that the chitin and beta-glucan links of the insoluble fraction of Aspergillus niger are linked to one another covalently, as mentioned, for example, by Stagg C M and Feather M S (Biochim. Biophys. (1973) Acta 320:64). Methods for determining the nature of the covalent linkage between the chitin and the beta-glucans have been described, for example, by Fontaine et al., for Aspergillus fumigatus (Fontaine T, Simenel C, Dubreucq G, Adam O, Delepierre M, Lemoine J Vorgias C E, Diaquin M & Latgé J P. (2000) Molecular organization of the alkali-insoluble fraction of Aspergillus fumigatus cell wall, J. Bio. Chem. 275:27594), and by Kollar et al., for the yeast Saccharomyces cerevisiae (Kollar R, Petrakovas E, Ashwell G, Robbins P & Cabib E. (1995) Architecture of the yeast cell wall, the linkage between chitin and beta(1,3)glucan, J. Biol. Chem. 270:1170).

The ratio of chitin to beta-glucan is between 95:5 and 5:95, preferably between 70:30 and 20:80, and more preferably between 70:30 and 25:75 (m/m). In another aspect the ratio of chitin to beta-glucan is between 10:90 and 50:50, which can be obtained by hydrolysing a chitin-glucan copolymer to obtain a higher glucan containing copolymer. The chitin part of the chitin-glucan copolymer is preferably composed of at least 85% of N-acetyl-D-glucosamine units and of at most 15% of D-glucosamine units, preferably of at least 90% of N-acetyl-D-glucosamine units and at most 10% of D-glucosamine units. The copolymer is generally in the form of a white to slightly brown powder. It is essentially insoluble in aqueous and organic solvents irrespective of the temperature and the pH. It is capable of swelling in aqueous media. It is hygroscopic, and can generally absorb approximately 10 times its mass in water.

Advantageously, the chitin-glucan copolymers according to the present invention are obtained by means of the process described above or in international application WO 03/068824 and French patent application FR 0507066 filed in the name of KITOZYME S.A. respectively on 12 Feb. 2003 and 4 Jul. 2005, which are entirely incorporated herein by way of reference. This process is described in particular in application FR 0507066, pages 18, line 14, et seq., of the application as filed. Aspergillus niger is preferably used as fungal source in this process.

The inventors mean, by “chitin-glucan hydrolysates”, the copolymers resulting from the controlled hydrolysis of the chitin-glucan copolymer extracted in particular from fungal cell walls, which also consist of covalently linked chitin links and beta-glucan links, with the ratio of chitin to beta-glucan preferably being between 50:50 and 10:90 (m/m), preferably between 60:40 and 15:85 (m/m). The hydrolysates have a molecular mass that is less than that of the chitin-glucan copolymer, resulting from partial hydrolysis. The chitin part of the chitin-glucan hydrolysates is preferably composed of at least 85% of N-acetyl-D-glucosamine units and of at most 15% of D-glucosamine units, preferably at least 90% of N-acetyl-D-glucosamine units and at most 10% of D-glucosamine units.

In a further aspect of the present invention, the inventors have discovered, surprisingly, that a chitin-glucan copolymer and its hydrolysates, belonging to a family of polysaccharides having a structure similar to that of chitosan, has blood-cholesterol-lowering properties equivalent to that of chitosan, although it is not essentially charged. This compound is readily obtained starting from fungal sources, for instance the mycelium of Aspergillus niger, of which it constitutes the insoluble part of the cell wall exoskeleton. The chitin-glucan copolymer and its hydrolysates also have other effects that are beneficial to the health, described in the present invention.

The invention relates in particular to the use of polysaccharides extracted from fungal sources and their hydrolysed derivatives, as pharmaceutical active agents or food supplements for improving human and animal health. Regular oral administration of the polysaccharides of the invention makes it possible in particular to prevent, treat or combat, especially, dyslipidemia, hypercholesterolemia and atherosclerosis, and to stimulate the antioxidant activities of the organism, to stimulate the immune system, to exert a hypoglycaemic action that is favourable in the case of diabetes, and to promote satiety so as to minimize metabolic syndrome. The polysaccharides of the invention are obtained from fungal sources according to an advantageous process that allows excellent purity and good reproducibility, with a large production capacity. The polysaccharides of the invention consist essentially of D-glucosamine units, and/or N-acetyl-D-glucosamine units and D-glucose units, in particular polymers of chitin (N-acetyl-D-glucosamine) and beta-glucan (D-glucose). The present invention also comprises any modifications of the polymer or copolymer, for example by grafting chemical functions onto the polymer, so as to improve the properties thereof.

Thus, according to a first aspect, the present invention relates to the use of at least one polysaccharide of fungal origin comprising predominantly a chitin-glucan copolymer, for the manufacture of a composition administered orally.

The present invention also relates to the use of at least one polysaccharide of fungal origin that is insoluble in an aqueous or organic medium, said polysaccharide comprising predominantly a chitin-glucan copolymer, for the manufacture of a composition administered orally.

The present invention also relates to the use of at least one polysaccharide of fungal origin comprising a polymer comprising beta-glucan linkages, said beta-glucan linkages consisting essentially of beta-glucan linkages in the 1,3-position, for the manufacture of a composition administered orally.

The present invention also relates to the use of at least one extract of fungal origin containing essentially a polysaccharide as defined above, for the manufacture of a composition administered orally. Advantageously, the polysaccharide comprises more than 70% of chitin-glucan polysaccharides by mass relative to the total mass of the extract of fungal origin, preferably greater than 85%.

The fungal extract can be obtained as described above. Sources of fungi which comprise glucans exist, but these units are water-soluble in particular, or comprise little or no chains of chitin structure, and do not therefore make it possible to obtain the polysaccharide of the present invention. The present invention covers all fungi which make it possible to obtain the chitin-glucan polymer defined in the present application.

Advantageously, a hydrolysate of the polysaccharide defined is used.

Advantageously, the chitin-glucan hydrolysate has a ratio of chitin to beta-glucans of between 60:40 and 15:85 (m/m).

Advantageously, at least 85% of the chitin part of the chitin-glucan copolymer is N-acetyl-D-glucosamine units, and at most 15% of this part is D-glucosamine units.

Thus, the invention makes it possible to provide a composition administered orally to a human being or an animal, preferably a mammal, for obtaining an effect chosen from the group consisting of an antioxidant, blood-cholesterol-lowering or blood-lipid-lowering effect, a stimulating effect on the immune system, a hypoglycaemic effect, in particular in the case of diabetes, a satiety effect, an effect which improves food transit, and an effect consisting in preventing and/or treating and/or combating a pathology chosen from the group consisting of dyslipidemia, atherosclerosis, obesity, an obesity-related disease, a cardiovascular disease, metabolic syndrome, diabetes and hyperuricemia.

The invention also relates to a pharmaceutical or food supplement composition comprising, as active ingredient, at least one polysaccharide or one extract of fungal origin, as defined above.

The present invention also relates to a method of treating, preventing or combating a pathology, in particular that mentioned above, comprising the oral administration of an effective amount of a composition comprising at least one polysaccharide as defined in the description above and hereinafter, to an individual needing the latter.

The present invention also relates to a method for decreasing the weight of or preventing or combating weight gain in a human being or an animal, and preferably a mammal. This method in particular concerns aesthetic care.

The present invention also relates to a method for promoting food transit.

Thus, the present invention relates to the use of a product of the present invention, for the manufacture of a composition intended in particular to be used in one of the methods described above or for exerting one of the effects described above and hereinafter.

Those skilled in the art can readily determine, by conventional methods, the effective amounts of the products of the invention to be used. An effective amount of between 0.001% and 100% by weight of the products according to the present invention, relative to the total weight of the composition to be administered, is advantageously used. If the products are administered in the form of gelatine capsules, granules, or tablets, they can be used pure or at any other concentration, accompanied by other active components or by excipients. If they are incorporated into foods, the concentration of product is less than 15%, and preferably less than 10%.

The products of the invention are generally formulated in the form of granules, tablets or gelatine capsules, or else incorporated into foods or into drinks.

### IN THE FIGURES

FIG. 1a represents the solid-state ¹³C-NMR spectrum of the alkali-insoluble fraction comprising a purified chitin-glucan polymer obtained after alkaline digestion of fungal biomass according to the first step in the method for isolating cell wall derivates of the present invention. The calculated chitin-glucan ratio is 41:59 (w/w).

FIG. 1b represents the solid-state ¹³C-NMR spectrum of the alkali-insoluble fraction comprising a purified chitin-glucan polymer obtained after alkaline digestion of fungal biomass obtained from shiitake (Lentinula).

FIG. 2 represents a X-ray scattering study of the alkali-insoluble fraction obtained after alkaline digestion of fungal biomass according to the first step in the method for isolating cell wall derivates of the present invention, whose chitin:glucan ratio was 38:62±3 (w/w).

FIG. 3 represents a X-ray scattering study of the alkali-insoluble fraction obtained after alkaline digestion of fungal biomass according to the first step in the method for isolating cell wall derivates of the present invention, whose chitin:glucan ratio was 85:15±8 (w/w).

FIG. 4 is a schematic view of the process for extracting the nonionic polysaccharides and of the various extracts that may be used. The FIG. 4 illustrates the main extracts that may be obtained from fungi.

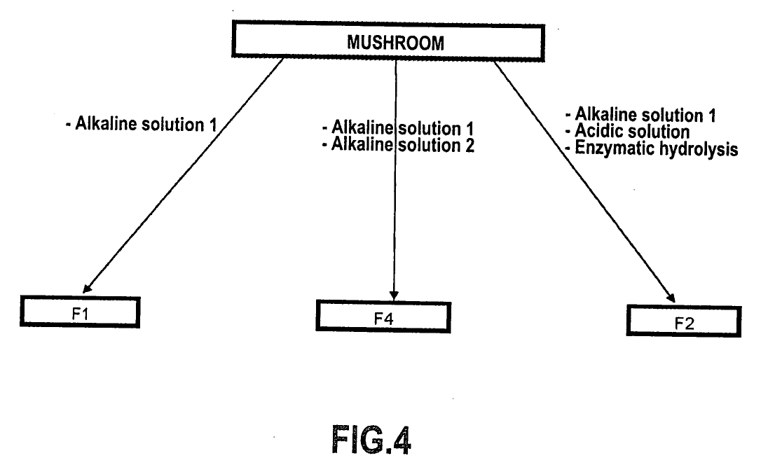

FIG. 5 represents the conditions for recording the solid-phase carbon 13 nuclear magnetic resonance (13C-NMR) spectrum of chitin-glucan F1 and hydrolysed chitin-glucan F4.

FIG. 6 represents the 13C-NMR spectrum of chitin-glucan F1 (batch 28).

FIG. 7 represents the 13C-NMR spectrum of the hydrolysed chitin-glucan F4 (batch 2).

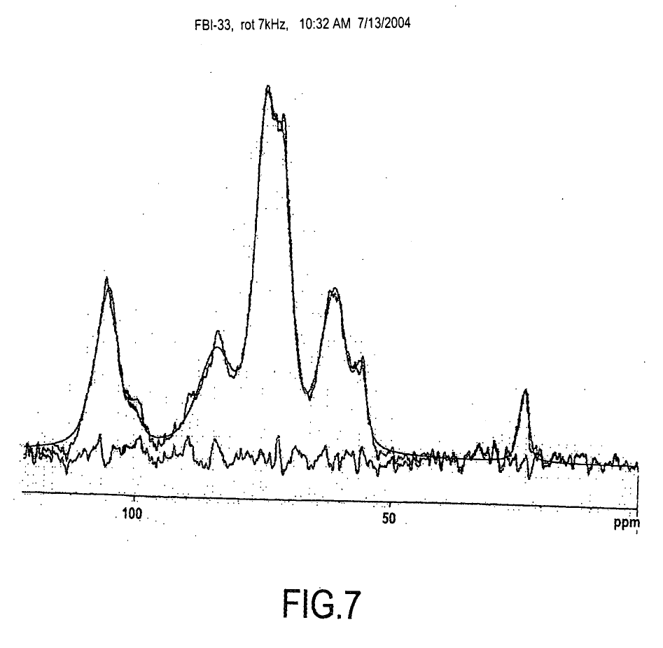

Other objectives, characteristics and advantages of the invention will emerge more clearly to those skilled in the art upon reading the explanatory description which refers to examples that are given only by way of illustration and cannot in any way limit the scope of the invention.

The examples are an integral part of the present invention and any characteristic that appears to be new in relation to any prior art based on the description taken in its entirety, including the examples, is an integral part of the invention in terms of its function and in terms of its generality.

Thus, each example has a general scope.

Furthermore, in the examples, all the percentages are given by weight, unless otherwise indicated, and the temperature is expressed in degrees Celsius unless otherwise indicated, and the pressure is atmospheric pressure unless otherwise indicated.

### EXAMPLES

### Example 1

### Alkaline Digestion of Aspergillus niger Mycelium

This example illustrates the first step in the method for isolating cell wall derivatives from fungal biomass according to the present invention. The biomass was obtained as side-product of a cultivation process for preparing citric acid using Aspergillus niger.

In this example, 995 g of the biomass containing 71% of water was collected and incubated in a reaction containing 2 liters of water and 93 g of sodium hydroxide pellets at room temperature, to reach a final biomass concentration of 3.4% (w/v). In this example, final concentration of NaOH comprised 10.6% (w/v) and the ratio of NaOH to biomass (dry weight) was 32%.

After 26 hours, the mixture was filtered to collect the insoluble fraction of the residual biomass, which was washed repeatedly until neutral pH was obtained. In this example, the dry mass of the insoluble fraction was 145 g. The analysis of this fraction by ¹³C-NMR in solid phase revealed that mainly a mixture of chitin and glucan polymers were obtained. In this example, the ratio of chitin to glucan, as calculated from the solid-state ¹³C-NMR spectrum was 52:48±15 (w/w).

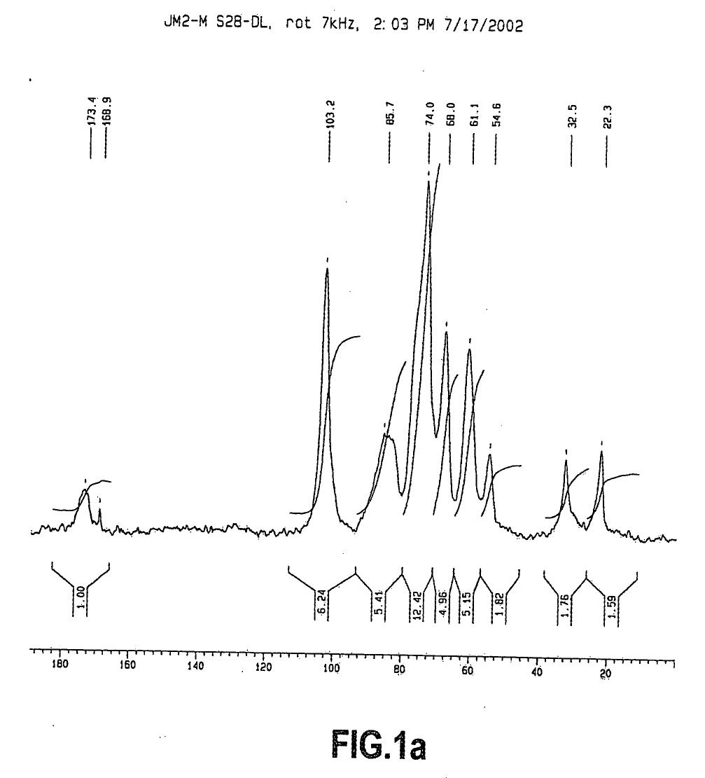

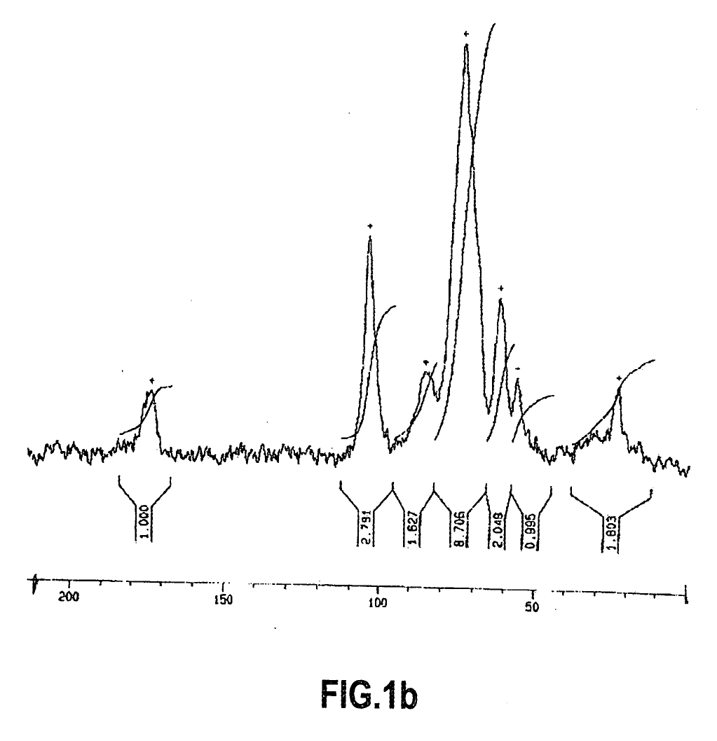

The chitin content in the insoluble fraction was determined by analysis of N-acetyl glucosamine released after hydrolysis of the insoluble fraction with chitinase and chitobiase enzymes, according to the method of Jeuniaux (“Chitine et chitinolyse: un chapitre de biologie moléculaire” 1963, Masson, Paris, 181) and Reissig et al. (J. Biol. Chem., 1955, 217:959). The chitin content was also determined from nuclear magnetic resonance analysis of carbon 13 in solid phase (¹³C-NMR) of the alkali-insoluble fraction obtained after alkaline digestion of the biomass. FIG. 1 represents the ¹³C RMN spectrum of the alkali-insoluble fraction comprising mainly a purified chitin-glucan polymer. After deconvolution and integration of the signals of the carbon atoms of N-acetyl-(D)-glucosamine and (D)-glucose units, the weight chitin:glucan ratio was calculated to be 41:59 (w/w).

### Example 2

### Alkaline Digestion of the Mycelium of Aspergillus niger

This example also illustrates the first step in the method for isolating cell wall derivatives from fungal biomass according to the present invention. The biomass was obtained as side-product of a cultivation process for preparing citric acid using Aspergillus niger.

In this example, the mycelium of Aspergillus niger was treated according to different conditions. Assays No. 1 to 4 were performed in a 10 L-reactor, and assays No. 5 to 6 in a 30 L-prototype reactor. Assays 1 to 5 were performed in one step, while assays 4′ and 6 were performed in two steps. In assay No 4′, the biomass was treated with a first NaOH solution (3.4%), then filtered and treated again in a second NaOH solution (2.8%). In assay No 6, the biomass was separated in two fractions successively placed in the reactor together with a low amount of NaOH followed by a higher amount of NaOH. Results are shown in Table 1.

TABLE 1

| | | | | | Dura- | m_{F} | ratio |
|---|---|---|---|---|---|---|---|
| | m_{mycellum} | C_{mycellum} | C_{NaOH} | T | tion | (% | Ch:Gl* |
| No | (g, dry) | % (w/v) | % (w/v) | (° C.) | (hours) | w/w) | (w/w) |
| 1 | 289 | 10.6 | 3.4 | 26 | 25 | 50 | 41:59 ± 3 |
| 2 | 505 | 9.2 | 1.5 | 25 | 26 | 57 | N/D |
| 3 | 580 | 10.7 | 1.5 | 40 | 26 | 57 | 44:56 ± 2 |
| 4 | 313 | 5.2 | 1.7 | 25 | 24 | 50 | 32:68 |
| 4′ | 485 | 10.6 | 3.4/2.8 | 25 | 24/6 | 40 | 37:63 |
| 5 | 496 | 2.9 | 2.0 | 25 | 22 | 49 | N/D |
| 6 | 446/446 | 2.9/2.9 | 2.0/4.0 | 25 | 22/18 | 49 | N/D |

m_{F}: proportion of final alkali-insoluble product to initial mycelium (dry mass)
N/D: not determined
*weight ratio of chitin to glucan as determined by ¹³C-NMR

An extraction procedure applied to the alkali-insoluble fraction collected in assay No 4, as described by Folch et al. (1957, J Biol Chem 224:497-509), showed an amount of lipophilic compounds of 6% of the initial dry weight.

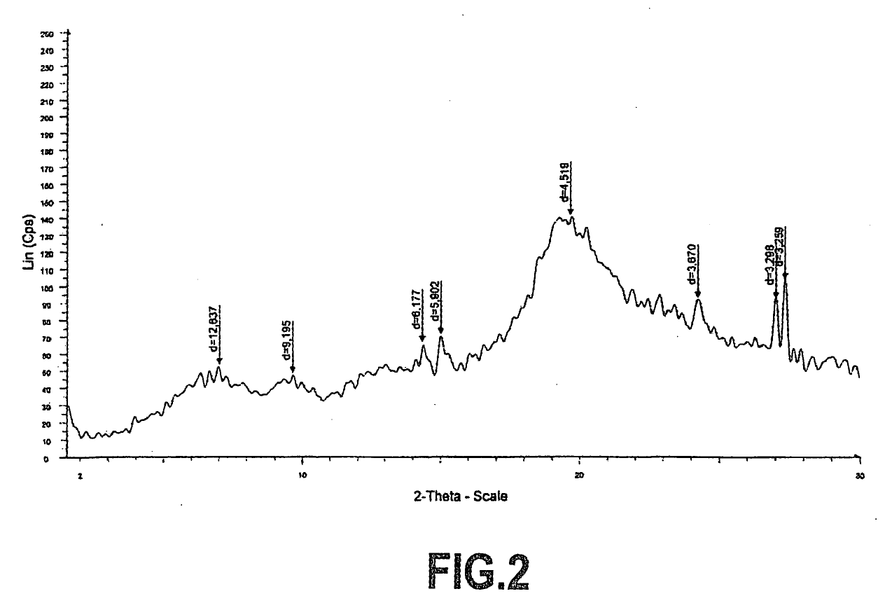

An X-ray scattering study (Siemens D5000, Cu-K_{□}, □=0.15406 nm, 2θ=1.5 á 30°, fente 1 mm, T=25° C.) of the alkali-insoluble fraction collected in assay No 4′, whose chitin-glucan ratio was 37:63 (w/w), showed a large scattering band at 2θ=20° (FIG. 2), indicating a semi-crystalline structure. The crystalline index can be calculated as proposed by Yinhai et al. (Chem. Mag. 2002, 4:27) or Struszczyk et al. (J. Appl Polym. Sci., 1987, 33:177-189), as an indication of the proportion of crystalline over amorphous regions in the compound. According to the calculation of Struszczyk, the index of cristallinity (CrI) of this chitin-glucan alkali-insoluble compound was 64%, a value much lower than that found for a chitin sample extracted from shrimp shells and analyzed by X-ray scattering in the same conditions (CrI=87%).

### Example 3

### Enzymatic β-Glucanases Preparations

This example illustrates a preferred procedure for testing several commercial preparations of β-glucanases for use in a method according to the present invention. β-glucanase activity can be quantified from standard curves established with pure reference β-glucanase enzymes that are reacted with standard β-glucan substrates. For instance for testing EC 3.2.1.6 β-glucanase activity, lichenase (Megazyme) or β-glucanase (Fluka) can be reacted with barley β-glucan substrate (Megazyme), for testing EC 3.2.1.39 β-glucanase activity, an endo-β-(1,3) enzyme (Megazyme, Fluka) can be reacted with pachyman or curdlan substrates (Megazyme), for testing EC 3.2.1.58 activity, an exo-β-glucanase (Megazyme) can be reacted with laminarin or schleroglucan substrates (Sigma, Megazyme), and for testing EC 3.2.1.75 β-glucanase activity, a β-(1.6) glucanase can be reacted with pustulan (Sigma). The β-glucanase activity (in U, unit) is defined as the amount of enzyme needed to release 1 μmole of glucose per minute, at 37° C. after incubation with the standard substrate, at the recommended pH.

The protein amount contained in the commercial enzyme preparation can be determined by the BCA (bicinchoninic acid) method, which relies on the reduction of Cu(II) ions into Cu(I) ions by proteins, in alkali conditions. Cu(I) ions are able to form a complex with BCA, whose absorption at 526 nm is proportional with the protein concentration (P K Smith et al. (1985) Anal. Biochem. 150, 76). The specific β-glucanase activity (in U/mg) exhibited by the commercial preparations is the ratio of the enzymatic activity and the mass of protein contained in the preparation.

Enzymatic preparations which contain one or several of the β-glucanase activities listed above are preferably tested (see Table 2). In the testing procedure, combinations of selected enzymes are preferably investigated for their ability to hydrolyse the β-glucan chains of the alkali-insoluble fraction of digested mycelium of Aspergillus niger.

TABLE 2
β-glucanase activities found in commercial enzyme preparations
(in U per mg of protein found in the preparation)

| Preparation | 3.2.1.6 activity | 3.2.1.39 activity | 3.2.1.58 activity |
|---|---|---|---|
| No | (U/mg protein) | (U/mg protein) | (U/mg protein) |
| 1 | 33 | 0 | 0 |
| 2 | 37 | 0 | 0 |
| 3 | 19 | 0 | 0 |
| 4 | 0 | 20 | 0 |
| 5 | 0 | 22 | 28 |
| 6 | 54 | 0 | 0 |
| 7 | 0 | 17 | 8 |
| 8 | 22 | 0 | 0 |

The β-glucanase hydrolysis reaction is preferably performed in the suspension of the alkali-insoluble fraction, which is obtained after alkaline treatment of biomass according to the present invention, at a pH preferably comprised between 4.0 and 7.0, and more preferably between 4.5 and 6.8. A mixture of β-glucanase preparations, for example a mixture of endo-β(1,3), exo-β-(1,3) and endo-β-(1,3)(1,4)-glucanase enzymes, is added to the suspension. To hydrolyse the β-glucan chains of the chitin-glucan extracted from an Aspergillus niger biomass, the proportion of β-glucanases, as expressed in unit of activity per mass of dry digested biomass, preferably ranges between 5 and 1500 U/g, and more preferably between 20 and 500 U/g. The digested biomass concentration preferably ranges between 0.5 and 15% (w/v), and more preferably between 2 and 8% (w/v). The preferred reaction temperature is below 40° C. The duration of the hydrolysis reaction ranges between 1 and 8 days, preferably below 5 days.

### Example 4

### Isolation of Cell Wall Derivates from Aspergillus niger Biomass According to the Invention

This example illustrates the isolation of cell wall derivatives from fungal biomass according to a method of the present invention. The biomass was obtained as side-product of a cultivation process for preparing citric acid using Aspergillus niger.

In this example, 3.3 kg of the biomass, containing 71% of water, 7.2 liters water and 320 g of NaOH pellets were placed in a reactor at room temperature. After 26 hours, the mixture was filtered to collect the insoluble fraction of the residual biomass, which was washed three times. The alkali-insoluble fraction was collected and suspended in 4 liters of water. The pH of the suspension was adjusted at 5.5 by addition of glacial acetic acid. To the acidified suspension, 13.2 g of the β-glucanase preparation No 5 (see Table 2), and 8.25 ml of the beta-glucanase preparation No 6 (see Table 2) were added. The reaction was carried out at 37° C. for 4 days. The suspension was then filtered, and the insoluble fraction washed in water and freeze-dried, to yield a mass of 34% of the initial chitin-glucan. For this example, the solid-state ¹³C-NMR spectrum of the compound revealed the presence of chitin and residual β-glucan polymers, with a chitin:glucan ratio of 94:6±14 (w/w).

### Example 5

### Beta-Glucanase Hydrolysis of a Chitin-Glucan Fraction of Aspergillus niger Biomass

This example illustrates the β-glucanase hydrolysis reaction performed in a method for isolating cell wall derivatives from fungal biomass according to the present invention.

In this example, the β-glucanase hydrolysis reaction was performed in different conditions, with variable amounts of commercial beta-glucanase preparations Nos 2, 5, and 6 (see Table 2), and for a duration of 5 days. The starting compound to be hydrolyzed was a freeze-dried chitin-glucan conjugate extracted from the mycelium of Aspergillus niger according to a method as described above, whose ratio of chitin to glucan was either 32:68 (assay No 1) or 38:62 (assays Nos 2 to 7). Results are shown in Table 3.

TABLE 3

| | ratio | | | | | | | ratio |
|---|---|---|---|---|---|---|---|---|
| | Ch:Gl₀ | m₀ | C_{Ch-Gl} % | Enzyme 5 | Enzyme 6 | Enzyme 2 | m_{F} | Ch:Gl_{F} |
| N^{o} | (w/w) | (g dry) | (w/v) | (mg/g) | (mg/g) | (mg/g) | (%) | (w/w) |
| 1 | 32:68 | 2.0 | 4.4 | 12.5 | 27.5 | 0 | 37 | 100:0 ± 5 |
| 2 | 38:62 | 2.7 | 8.6 | 13 | 28 | 0 | 37 | 85:15 ± 8 |
| 3 | 38:62 | 1.9 | 4.2 | 56 | 33 | 0 | 38 | 96:4 ± 22 |
| 4 | 38:62 | 1.9 | 4.2 | 29 | 0 | 0 | 48 | 104:0 ± 23 |
| 5 | 38:62 | 1.9 | 4.2 | 12 | 33 | 0 | 39 | 105:0 ± 23 |
| 6 | 38:62 | 1.9 | 4.2 | 12 | 0 | 160 | 41 | 96:4 ± 21 |
| 7 | 38:62 | 1.9 | 4.2 | 6 | 16 | 0 | 46 | 80:20 ± 9 |

m_{F}: proportion of final alkali-insoluble product to initial mycelium (dry mass);
ratio Ch:Gl_{F:} ratio of chitin to glucan as determined by ¹³C-NMR.

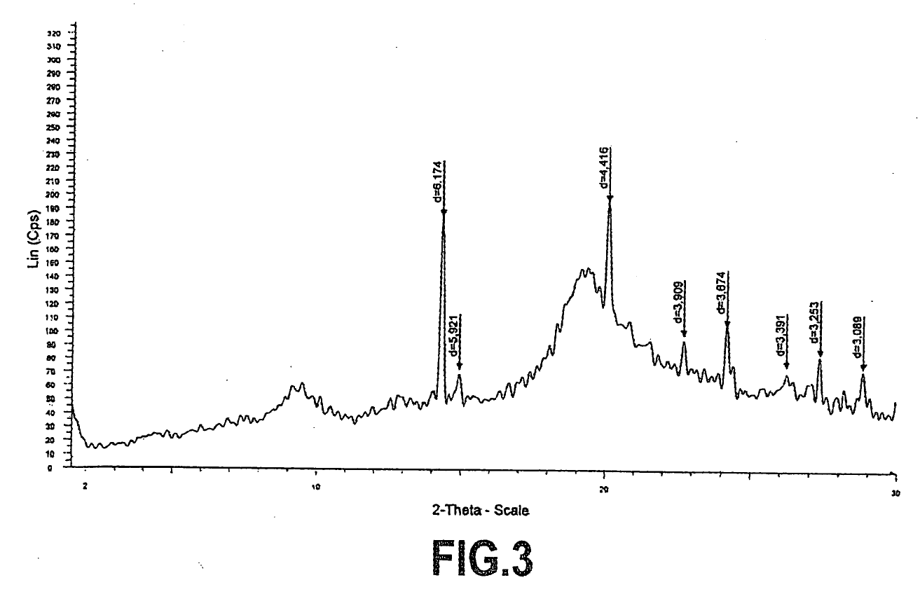

The X-ray scattering study (Siemens D5000, Cu-K_{α}, λ=0.15406 nm, 2θ=1.5 to 30°, fente 1 mm, T=25° C.) of the chitin-rich alkali-insoluble fraction of assay No 2, whose chitin-glucan ratio was 85:15±8 (w/w), showed a large scattering band at 2θ=20° (FIG. 3). In this example, the crystalline index of the compound calculated according to Struszczyk et al. (J. Appl. Polym. Sci. (1987) 33, 177-189) was 67%, while it is 87% for chitin extracted from shrimp shells.

### Example 6

### Preparation of Fungal Chitosan from Chitin

This example illustrates the preparation of chitosan from chitin obtained after β-glucanase hydrolysis of a chitin:glucan fraction of Aspergillus niger mycelium.

4 g of the insoluble fraction obtained by beta-glucanase hydrolysis in example 4, 40 g of NaOH and 40 ml of water were placed at 120° C. for 1 hour. The obtained suspension was then centrifuged, filtered and washed until low conductivity. The insoluble fraction was suspended in 200 ml of water, and acetic acid was added to reach a pH of 3.5. After 12 hours, the solution was filtrated, and the filtrate was collected. In this example, the pH of the filtrate was adjusted to 9.5 by addition of ammonium hydroxide to promote the precipitation of chitosan. After centrifugation, washing and freeze-drying, 1 g of the acid-soluble fraction was obtained. In this example, the solid-state ¹³C-NMR spectrum of the compound revealed that the acid-soluble fraction was pure chitosan, with no residual β-glucan chains. The proportion of N-acetyl-D-glucosamine was 14 mol % and the viscosimetric molecular weight was around 20,000 Da, as measured by Ubbelohde capillary viscosimetry.

### Example 7

### Preparation of Fungal Chitosan Chloride from Chitin

This example illustrates the preparation of chitosan chloride from chitin obtained after beta-glucanase hydrolysis of a chitin-glucan fraction of Aspergillus niger mycelium

In this example, 60 g of a chitin-rich insoluble fraction obtained by β-glucanase hydrolysis as e.g. in example 4, 300 g of NaOH, 6 g of sodium boron hydride, and 300 g of water were placed at 120° C. for 1 hour. The obtained suspension was then centrifuged, filtered and washed until low conductivity. The insoluble fraction was suspended in 200 ml of acetic acid 0.5 M. After 12 hours, the solution was filtrated, and the filtrate was collected. In this example, the pH of the filtrate was adjusted to 9.5 by addition of ammonium hydroxide to promote the precipitation of chitosan. After centrifugation and washing, the acid-soluble fraction was solubilized in 28 ml of HCl 1N at pH 3.6. The solution was then freeze-dried, yielding 6 g of chitosan under the ammonium chloride form. The proportion of N-acetyl-glucosamine is 17 mol % and the viscosimetric molecular weight is around 20,000 Da.

### Example 8

### Preparation of High Molecular Weight Chitosan with Low Degree of Acetylation by Enzymatic Deacetylation of Chitin

In this example, commercial chitin from shrimp shells was treated in different conditions in order to yield chitosan. First, it was treated with a strong alkaline solution of NaOH at variable concentration and NaOH:chitin ratio, in order to transform chitin into a gel form and to induce partial deacetylation of N-acetyl-glucosamine into glucosamine units. Then, the partially deacetylated chitin was filtrated, washed, and resuspended in solution of sodium phtalate (10 mM) at pH 5.5 to yield a chitin concentration of 5% (w/v). Subsequently, a recombinant chitin deacetylase enzyme (rCDA) was added, to reach a rCDA:chitin ratio of 5:1000, and the suspension was placed at 37° C. for 5 days. To estimate the extend of deacetylation promoted by rCDA, the released acetic acid was assessed. The suspension was filtered, and the alkali-insoluble fraction was washed with water and dried. It was then solubilized in a solution of acetic acid, filtrated, and then pH was raised by addition of ammonium hydroxide to promote the precipitation of chitosan chains. The precipitate was then washed and dried. Results of the assays are represented in Table 4.

TABLE 4

| | t | T | C_{NaOH} % | C_{chitin} % | NaOH:chitin | m_{F} | DA* | | D_{DA}** |
|---|---|---|---|---|---|---|---|---|---|
| No | (min) | (° C.) | (w/w) | (w/v) | (w/w) | (%) | (mol %) | Aspect | (%) |
| 1 | 30 | 100 | 30 | 7 | 6 | 100 | 87 | X | <10 |
| 2 | 30 | 100 | 40 | 8 | 8 | 76 | 64 | X | <10 |
| 3 | 30 | 80 | 50 | 10 | 10 | 75 | 75 | X | <20 |
| 4 | 30 | 80 | 50 | 10 | 10 | 88 | 82 | X | <20 |
| 5 | 60 | 80 | 50 | 10 | 10 | 45 | 68 | Gel | >20 |
| 5′ | 60 | 80 | 50 | 10 | 10 | 60 | 57 | Gel | >20 |
| 6 | 30 | 100 | 50 | 4 | 25 | 46 | 72 | Gel | <20 |
| 7 | 30 | 110 | 50 | 10 | 10 | 51 | 53 | Gel | >20 |
| 8 | 60 | 110 | 50 | 10 | 10 | 43 | 39 | Gel | >20 |

m_{F}: mass of chitin after the treatment in alkali;
*DA: degree of acetylation of chitin after alkaline treatment;
**D_{DA}: difference in degree of acetylation of the acid-soluble fraction of alkali-treated chitin before and after reaction with rCDA (efficiency of the CDAse reaction)

In this example, the efficiency of the rCDA enzyme, as shown by the difference in DA before and after reaction with rCDA (D_{DA}), depended on the previous alkali treatment, mainly the NaOH concentration and the temperature. As observed in assays Nos 5 to 8 of this example, chitin preferably is in a gelled form when the alkali concentration is above 50% (w/w), and is preferably sufficiently pre-deacetylated, in order to allow the rCDA to catalyse the deacetylation reaction.

### Example 9

### Preparation of High Molecular Weight Chitosan by Enzymatic Deacetylation of Chitosan

This example illustrates the preparation of chitosan having a high molecular weight and a low degree of acetylation by enzymatic deacetylation of chitosan. Various chitosan samples characterized by their initial viscosimetric molecular weight (Mv₀) and degree of acetylation (DA₀) were reacted with the recombinant chitin deacetylase (rCDA), in order to decrease the degree of acetylation to a lower value (DA_{F}). In this series of assays, the reaction medium was either a non buffered solution of chlorhydric 1N (assays No 1 to 4) or formic acid 1N (assays Nos 5 to 10) or a buffered solution of formic acid 1N with sodium phtalate or (No 10) or glutamate (No 11) at varying pH. In this example, the ratio of rCDA to chitosan was either 1:1000 (No 4) or 5:1000.

Chitosan was then recovered as described in the above-given examples, by precipitation at pH above 7.0. Results of the assays are represented in Table 5. For all samples, the final viscosimetric molecular weight was unchanged.

TABLE 5

| | DA₀ | Mv₀ | Cp | | | T | T | C_{CDA} | DA_{F} |
|---|---|---|---|---|---|---|---|---|---|
| No. | mol % | kDa | (% w/v) | Solution | pH | (hrs) | (° C.) | (g/kg) | (mol %) |
| 1 | 19 | 500 | 0.5 | HCl 1N | 3.8 | 6 | 20 | 5 | 11 |
| 2 | 19 | 500 | 0.5 | HCl 1N | 4.6 | 6 | 20 | 5 | 21 |
| 3 | 19 | 500 | 0.5 | HCl 1N | 3.6 | 3 | 20 | 5 | 12 |
| 4 | 19 | 500 | 1.0 | HCl 1N | 3.8 | 6 | 20 | 1 | 13 |
| 5 | 19 | 500 | 0.5 | Formic acid | 3.8 | 6 | 20 | 5 | 12 |
| | | | | 1N | | | | | |
| 6 | 19 | 500 | 0.5 | Formic acid | 3.8 | 6 | 50 | 5 | 20 |
| | | | | 1N | | | | | |
| 7 | 17 | 142 | 0.5 | Formic acid | 3.8 | 6 | 20 | 5 | 11 |
| | | | | 1N | | | | | |
| 8 | 12 | 225 | 0.5 | Formic acid | 3.8 | 6 | 20 | 5 | 10 |
| | | | | 1N | | | | | |
| 9 | 13 | 245 | 0.5 | Formic acid | 3.8 | 6 | 20 | 5 | 10 |
| | | | | 1N | | | | | |
| 10 | 19 | 500 | 0.5 | Phtalate 10M, | 4.6 | 6 | 20 | 5 | 11 |
| | | | | formic acid | | | | | |
| | | | | 1N | | | | | |
| 11 | 19 | 500 | 0.5 | Glutamate | 4.8 | 6 | 20 | 5 | 14 |
| | | | | 10M, | | | | | |
| | | | | formic acid | | | | | |
| | | | | 1N | | | | | |

### Example 10

### Preparation of a Porous Support Comprising Chitosan

Chitosan obtained according to a method of the present invention can be used for the preparation of films or porous objects, whose size of the pores is controlled.

For example, particles of gauged size consisting of water-soluble molecules (e.g. sodium chloride) can be mixed with chitosan in an acid solution.

Then this chitosan matrix is solidified by solvent evaporation or freeze-drying. The particles are eliminated by washing to generate the pores.

Porous matrices comprising chitosan can also be prepared by means of polymer/solvent phase separation of the liquid to solid or liquid to liquid type, which were thermically induced. For example, chitosan is dissolved in a solvent such as a concentrated or diluted organic acid, for example acetic acid or formic acid, and is subsequently frozen at a temperature lower than the temperature of solidification of the solvent (freezing point), and then freeze-dried. The pores are generated at the place of the solvent crystals, crystals that are formed at the time of freezing by a mechanism of transition from liquid to solid phase. A transition from liquid to liquid phase can also be induced by dissolving chitosan in a solvent mixture of a solvent and a non solvent (both able to be freeze-dried).

The solvent may be a concentrated organic acid such as acetic or formic acid. The size and the distribution of the pores depend on the mechanism of transition from polymer/solvent phase.

Application of Chitin-Glucan Copolymers as Technological Additive

The processes for clarifying and treating the food-grade liquids by using extracts obtained from fungal biomasses are performed in the case of grape musts and red, white, rose and natural sweet wines, but are purely illustrative and do not in any way limit the scope of the protection sought. In particular, wines are beverages whose composition is very complex and “fragile”. By performing the treatment of wines with the fungal extract according to the present invention, the diversity of liquids that may be treated is illustrated.

In the examples that follow, reference is made to fungal extracts F1, F2 and F4. These extracts are obtained in the following manner:

To prepare the fungal extract F1, a mass of 50 kg (dry weight) of wet Aspergillus niger biomass is suspended in a hydrochloric acid solution at a concentration of 1%, and then filtered. The solid matter is then suspended in a 0.25% sodium hydroxide solution, and then filtered. The solid matter is washed 4 times with water, and then dried. It is then suspended in ethanol, and then filtered and dried. About 20 kg of material F1 are obtained.

To prepare the fungal extract F2, a mass of 50 kg (dry weight) of wet Aspergillus niger biomass is suspended in a hydrochloric acid solution at a concentration of 1%, and then filtered. The solid matter is then suspended in a 0.25% sodium hydroxide solution, and then filtered. The solid matter is washed 4 times with water and then suspended in water. Glacial acetic acid is added to pH 5.3. 1 kg of enzymatic preparation rich in beta-glucanase is added, and the reaction is continued for 4 days at room temperature. The material is filtered off and then suspended in ethanol in the presence of potassium hydroxide, at 60° C. for 1 hour, and then filtered. The material is then suspended in ethanol, and then filtered off. The material consisting of the chitin-rich copolymer is then dried. About 8 kg of material F2 are obtained.

To prepare the fungal extract F4, a mass of 50 kg (dry weight) of wet Aspergillus niger biomass is suspended in a hydrochloric acid solution at a concentration of 1%, and then filtered. The solid matter is then suspended in a 0.25% sodium hydroxide solution, and then filtered off. The material is then placed in contact with a 30% concentrated sodium hydroxide solution at 100° C. for 2 hours. It is then washed several times with water, then suspended in ethanol, and then filtered off and dried. About 5 kg of material F4 are obtained.

Thus, the extract F1 may be obtained by treating fungi with an alkaline solution, preferably at a concentration of less than 10%. The extract F4 is obtained by treating fungi with a first alkaline solution at a concentration preferably of less than 10%, followed by treatment with a second alkaline solution at a concentration preferably greater than 10%. The extract F2 is obtained by treating fungi with an alkaline solution, treatment with an acidic solution and treatment with an enzyme with beta-glucanase activity.

The molecular and purity characteristics of the fungal extracts are given in Table 1.

TABLE 6
Molecular and purity characteristics of the fungal extracts

| | | | Purity of the |
|---|---|---|---|
| | | | chitin-glucan |
| | Chitin as % of | Beta-glucan | copolymer |
| | the chitin-glucan | as % of the chitin- | as mass % of the |
| | copolymer | glucan copolymer | fungal extract |
| F1 | 46 | 54 | 97 |
| F4 | 26 | 74 | 98 |
| F2 | 78 | 22 | 97 |

In the examples that follow, reference is also made to products C1 and F7, which are both chitosans, used in the form of solid powder.

C1 is a commercially available chitosan of crustacean origin (Chitoclear LV, Primex, 99% purity, 16 mol % degree of acetylation, viscometric-average molecular mass 70 000).

F7 is a chitosan of fungal origin, obtained by deacetylation of a chitin-rich fungal extract (KitoZyme, 97% purity, 10 mol % degree of acetylation, viscometric-average molecular mass 10 000).

### Example 11

### Stabilization of Red and White Wine Musts with a Fungal Extract

Red and white wine musts were treated by adding the fungal extracts F1, F2, F7 and C1 during fermentation. The fermentation was performed with a yeast S. cerevisiae Lalvin BM 45 at 22° C. The addition of a fungal extract at a dose of 10 or 50 g/hl is performed after 6 days, except for the control wine. The fermentation is stopped 3 days later.

For this study, 110 liters of red grape must of Grenache noir grape variety and 110 liters of white grape must of Grenache blanc grape variety obtained from the INRA de Pech Rouge, Gruissan (Aude) were used. These 110 liters of red or white must were separated into 11 tanks of 10 liters each. Each tank was then supplied with yeast using the yeast Saccharomyces cerevisiae, Lalvin BM 45, with rehydration in warm water while incorporating nutrients (thiamine 60 mg/hl; ammonium phosphate 200 mg/l).

The fermentation is performed at a temperature of 22° C. and lasts for 9 days. After 6 days of fermentation, a dose of 10 g/hl or 50 g/hl of one of the following compounds was added to the tanks:
fungal extract F2
fungal extract F1
chitosan F7 or C1
fungal extract F4

The control tank receives no treatment. Fermentation is continued until it stops after depletion of the sugars arising from the treatments (3 days). During the fermentation, the temperature and density are measured daily in order to monitor the fermentation process.

The turbidity, the protein content and the concentration of polyphenolic compounds are assayed on the wines at the end of fermentation, as explained below.

The turbidity is measured by turbidimetry, official method of the OIV (Oeno Resolution 4/2000—wine turbidity) and expressed in NTU (nephelometric turbidity units). The decrease in turbidity is measured by the official method of the OIV, Oeno Resolution 4/2000. This is a measurement of the reduction of the transparency of a liquid due to the presence of undissolved matter. The machine used is a Hach brand 2100N turbidimeter. The unit of measurement of the turbidity index, NTU, corresponds to a measurement of the light scattered by a standard formazine suspension at an angle of 90° relative to the direction of the incident beam. The measurement should be performed at a temperature of between 15 and 25° C.

The protein content is assayed by the Bradford method, expressed in mg of proteins/l. The assay used for the removal of proteins is performed by determining the protein-based nitrogen content in the wines (Bradford method, Bradford M M, Anal. Biochem., 1976, 72, 248-254). The Bradford method consists in reacting a sample of must with a reagent, the Bradford reagent. The protocol proceeds in two steps: in a first stage, 10 ml of must or wine are mixed with 10 ml of acetone. This mixture is cooled to −20° C. for 30 minutes. This mixture is then centrifuged at 4000 rpm approximately for 10 minutes. Next, the acetone is separated out and the precipitate is redissolved with 1 ml of 0.1M sodium hydroxide and 4 ml of Bradford reagent. This mixture is adjusted to 10 ml with distilled water. After 15 minutes, the absorbance is read at 595 nm against a blank containing 1 ml of 0.1M sodium hydroxide, 4 ml of Bradford reagent and 5 ml of distilled water. The concentration obtained is expressed in mg equivalent of bovine serum albumin/l.

The total polyphenolic compound content (TPC) is determined by the Folin-Ciocalteu method, expressed in mg of gallic acid equivalent (mg eq GAE/l). The total polyphenolic compounds are analyzed according to the method described in “Singleton V L, Drapper D E, The transfer of phenolic compounds from grapes seeds into wine, J. Enol. Vitic. 1964, 15, 131-145”. The Folin-Ciocalteu method is based on a calorimetric reaction whose response depends on the phenolic compounds present in the analyzed phenolic extracts. The development of the coloration depends on the number of hydroxyl groups or of potentially oxidizable groups. The phenolic groups must be in phenoxide form to result in the oxidation of the phosphotungstic and phosphomolybdic anions present in the reagent. In practice, 200 μl of 5-fold diluted red wine, or 200 μl of white wine are introduced into a 20 ml flask. 1 ml of Folin-Ciocalteu reagent, 12 ml of distilled water and 4 ml of Na₂CO₃ (at 20%) are added. The mixture is adjusted to the graduation mark with distilled water. The absorbance is determined after 30 minutes using a spectrophotometer at 765 nm, the total phenolic compound content being calculated relative to a calibration curve established with gallic acid.

TABLE 7

| | Red wine | | | White wine | | |
|---|---|---|---|---|---|---|
| | Turbidity | Proteins | | Turbidity | Proteins | |
| Variation | % | % | TPC % | % | % | TPC % |
| Control | 3670 NTU | 184 | 2341 | 130 NTU | 120 | 280 |
| | | mg/l | GAE/I | | mg/l | GAE/I |
| F1 50 g/hl | −93% | −91% | −33% | −96% | −83% | −64% |
| F1 10 g/hl | −93% | −77% | −41% | −90% | −80% | −55% |
| F2 50 g/hl | −89% | −75% | −40% | −97% | −83% | −54% |
| F2 10 g/hl | −90% | −66% | −37% | −96% | −86% | −70% |
| F7 50 g/hl | −91% | −80% | −27% | −99% | −88% | −33% |
| F7 10 g/hl | −90% | −72% | −26% | −98% | −92% | −40% |
| C1 50 g/hl | −92% | −97% | −33% | −99% | −87% | −34% |
| C1 10 g/hl | −94% | −74% | −46% | −98% | −86% | −46% |

*TPC: total phenolic compounds; variation relative to the control that has undergone the same steps

It results clearly from Table 2 that the wines are stabilized, without all of the polyphenols being removed.
In Examples 12, 13 and 14 below, a red wine (Example 12), a white wine (Example 13) and a rose wine (Example 14) were treated by adding extracts F4 and C1 at doses of 10, 50 and 200 g/hl, with a contact time of 24 hours, with or without gentle stirring.

For this study, we used:

a traditional red wine, vintage 2002, containing grenache, syrah and carrignan grape varieties. This wine is conditioned in 750 ml bottles. Its content of total phenolic compounds is 1850 mg GAE/l;

a chardonnay paradoxe blanc white wine, vintage 1999 from vineyard of Virginie Castel. This wine is conditioned in 750 ml bottles. This wine has the characteristic of being vinified like a red wine including a maceration phase and a temperature increase. Thus, the contents of catechin dimers are very much higher than those of a white wine that has undergone a standard vinification. Its content of total phenolic compounds is 1000 mg GAE/l;

a traditional rosé wine, vintage 2002. This wine is conditioned in 750 ml bottles. Its content of total phenolic compounds is 365 mg GAE/l.

For each test, for example on red wine, all the 750 ml bottles of wine are homogenized in a first stage, and are then divided into 100 ml aliquots. Compounds F4 or C1 are added to each of these aliquots, at a dose of 10 g/hl, 50 g/hl or 200 g/hl with a contact time of 24 hours, with or without gentle stirring, at room temperature.

Assay Used for Assaying the Tannins in Examples 12a, 12b, 13a and 13b

For the determination, fractionation of the phenolic material is performed on a column, by qualitative estimation of the coloring matter (according to Bourzeix et al., 1979). This estimation is performed by column fractionation. Three fractions are separated. These various fractions change in the course of the aging of the wine:

1—The free anthocyan monomers by means of a mixture of 999 vol methanol and 1 vol 12N HCl. These monomers are collected in 20 ml of eluate and the optical density is then measured at 538 nm in order to evaluate the content of this fraction.

2—The red polymers, i.e. the forms weakly condensed by means of a mixture of formic acid and water (1/1 by vol). These red polymers are collected in 20 ml of eluate and the optical density is then measured at 525 nm in order to evaluate the content of this fraction.

3—The yellow and brown polymers, i.e. the forms condensed by means of pure formic acid. These yellow and brown polymers are collected in 20 ml of eluate and the optical density is then measured at 525 nm in order to evaluate the content of this fraction.

Assay Used for Assaying Tannins in Examples 12c and 12d

Analysis of wine tannins (Ribéreau-Gayon P, Glories Y, Maujean A, Dubourdieu D, 1998 Traité d'oenologie 2. chimie du vin stabilization et traitement [Oenology treatise 2. wine chemistry stabilization and treatment], Dunod, Paris p 203)

This method is also known as a tannin assay, the LA method or the proanthocyanin tannin assay method. It is based on the Bate-Smith reaction. Heating procyanidins in acidic medium leads to the cleavage of certain bonds and to the formation of carbocations that become partially transformed into cyanidin if the medium is sufficiently oxidizing. To do this, the procedure comprises the preparation of two samples each containing 4 ml of wine diluted to 1/50, 2 ml of water and 6 ml of pure (12N) HCl; one of the tubes is heated on a waterbath at 100° C. for 30 minutes and 1 ml of 95% ethanol is added thereto to dissolve the apparent red color (D2); the other is not heated, but receives 1 ml of 95% ethanol (D1). The difference is measured

Δd=D2−D1 of the optical density at 550 nm over a 10 mm optical path; by comparison with a reference solution of procyanidin oligomers, the following concentration is obtained:
LA(g/l)=19.33×Δd
Assay Used to Analyze the Color Intensity, the Shade, the Radiance and the Color Composition of the Wine in Examples 12a, 12b, 12c, 12d, 13a and 13b

Analysis of the coloring matter of wine is performed according to “Etude de la couleur du vin [Study of the color of wine]” (Ribéreau-Gayon P, Glories Y, Maujean A, Dubourdieu D, 1998 Traité d'oenologie 2. chimie du vin stabilization et traitement [Oenology treatise 2. Wine chemistry stabilization and treatment], Dunod, Paris p 206-207). This study is defined by 4 parameters:

The color intensity represents the strength of the color.
CI=OD 420+OD 520+OD 620

The shade corresponds to the level of change of the color towards orange. It increases in the course of aging of the wine.
T=OD420/OD520

The color composition corresponds to the contribution in the form of percentage of each of the three components toward the overall color.
OD 420(%)=(OD 420/CI)×100
OD 520(%)=(OD 420/CI)×100
OD 620(%)=(OD 420/CI)×100

The radiance is related to the shape of the spectrum. The more dominant and bright the red color of the wine, the higher this parameter.
dA(%)=(1−(OD 420+OD 620/2×OD 520))×100

### Example 12

### Treatment of Finished Wines with an Extract of Fungal Biomass, or an Extract of Crustacean Biomass: Red Wines

This example serves particularly to illustrate the following effects:

Improvement of the clarity

Improvement of the color composition of the wine

Variation of the pH of the wine

Removal of some of the polyphenols

Conservation of the phenolic material of the wine

### Example 12a

### Characteristics of Red Wines after Treatment without Stirring with a Contact Time of 24 Hours

TABLE 8
Variations of pH and of total phenolic compounds content in control
and treated red wines, after addition of products F4 and C1
without stirring, contact time of 24 hours

| | | Variation* | TPC | Variation |
|---|---|---|---|---|
| | | (%) | (mg eq GAE/I) | (%) |
| Control | 3.75 | | 1850 | |
| F4 10 g/hl | 3.73 | −0.5% | 1799 | −2.74% |
| F4 50 g/hl | 3.73 | −0.5% | 1784 | −3.56% |
| F4 200 g/hl | 3.77 | +0.5% | 1697 | −8.22% |
| C1 10 g/hl | 3.74 | −0.3% | 1834 | −0.82% |
| C1 50 g/hl | 3.77 | +0.5% | 1672 | −9.59% |
| C1 200 g/hl | 3.81 | +1.6% | 1672 | −9.59% |

*relative to the control

It results clearly from Table 8 that the pH of the wine varies negligibly and that all the polyphenols are conserved with a negligible variation.

TABLE 9
Fractionation of the phenolic material of control and treated red wines,
after adding the products F4 and C1 without stirring, contact time
of 24 hours

| | Mono- | Varia- | Red | Varia- | Brown | Varia- |
|---|---|---|---|---|---|---|
| | mers | tion | polymers | tion | polymers | tion |
| | % | % | % | % | % | % |
| Control | 37.53 | | 39.50 | | 22.96 | |
| F4 50 g/hl | 37.00 | −1.4% | 42.10 | +6.2% | 20.90 | −9.9% |
| F4 200 | 36.44 | −3.0% | 42.85 | +8.0% | 20.70 | −10.8% |
| g/hl | | | | | | |
| C1 50 g/hl | 36.68 | −2.3% | 40.20 | +1.7% | 23.11 | +0.7% |
| C1 | 36.92 | −1.7% | 41.02 | +3.6% | 22.05 | −4.4% |
| 200 g/hl | | | | | | |

It results clearly from Table 9 that a rearrangement in favor of the red polymers is obtained.

TABLE 10
Color intensity, shade and radiance of control and treated red wines,
after addition of the products F4 and C1 without stirring, contact time of
24 hours

| | | Varia- | | Varia- | | Varia- |
|---|---|---|---|---|---|---|
| | Color | tion | | tion | | tion |
| | intensity | % | Shade | % | Radiance | % |
| Control | 1.06 | | 1.05 | | 27.42 | |
| F4 10 g/hl | 0.93 | −12.6% | 0.96 | −8.6% | 31.04 | +13.2% |
| F4 50 g/hl | 0.99 | −6.9% | 0.97 | −7.6% | 29.56 | +7.8% |
| F4 200 | 0.89 | −16.4% | 0.94 | −10.5% | 30.96 | +12.9% |
| g/hl | | | | | | |
| C1 10 g/hl | 0.95 | −10.7% | 0.99 | −5.7% | 30.32 | +10.6% |
| C1 50 g/hl | 0.88 | −17.3% | 0.96 | −8.6% | 32.66 | +19.1% |
| C1 200 | 0.87 | −18.2% | 0.95 | −9.5% | 31.19 | +13.8% |
| g/hl | | | | | | |

It results clearly from Table 10 that the color intensity and the shade decrease, whereas the radiance is improved.

TABLE 11
Color composition of control and treated red wines, after adding the
products F4 and C1 without stirring, contact time of 24 hours

| | OD | Varia- | OD | Variation | OD | Variation |
|---|---|---|---|---|---|---|
| | 420 nm | tion % | 520 nm | % | 620 nm | % |
| Control | 42.60 | | 40.80 | | 16.50 | |
| F4 10 g/hl | 40.64 | −4.6% | 42.03 | +3.0% | 17.32 | +5.0% |
| F4 50 g/hl | 40.50 | −4.9% | 41.51 | +1.7% | 17.97 | +8.9% |
| F4 200 | 39.63 | −7.0% | 42.00 | +2.9% | 18.35 | +11.2% |
| g/hl | | | | | | |
| C1 10 g/hl | 41.67 | −2.2% | 41.78 | +2.4% | 16.54 | +0.2% |
| C1 50 g/hl | 40.90 | −4.0% | 42.61 | +4.4% | 16.47 | −0.2% |
| C1 | 40.00 | −6.1% | 42.06 | +3.1% | 17.93 | +8.7% |
| 200 g/hl | | | | | | |

It results clearly from Table 11 that a very slight rearrangement of the color appears: the optical density at 420 nm (yellow-green) decreases while the optical density at 520 nm (purple) and the optical density at 620 nm (blue-green) increase.

### Example 12b

### Characteristics of Red Wines after Treatment with Gentle Stirring, with a Contact Time of 24 Hours

TABLE 12

| | | Variation* | TPC | Variation* |
|---|---|---|---|---|
| | | (%) | (mg eq GAE/I) | (%) |
| Control | 3.77 | | 1850 | |
| F4 10 g/hl | 3.71 | −1.6% | 1807 | −2.3% |
| F4 50 g/hl | 3.73 | −1.1% | 1850 | 0% |
| F4 200 g/hl | 3.75 | −0.5% | 1744 | −5.7% |
| C1 10 g/hl | 3.76 | −0.3% | 1522 | −12.3% |
| C1 50 g/hl | 3.78 | +0.3% | 1585 | −14.3% |
| C1 200 g/hl | 3.87 | +2.7% | 1585 | −14.3% |

*relative to the control

It results clearly from Table 12 that the pH of the wine varies negligibly, and the polyphenols are conserved with negligible variation.

TABLE 13
Fractionation of the phenolic material of control and treated red
wines, after adding the products F4 and C1 with stirring, contact time of
24 hours

| | Mono- | Varia- | Red | Varia- | Brown | Varia- |
|---|---|---|---|---|---|---|
| | mers | tion | polymers | tion | polymers | tion |
| | % | % | % | % | % | % |
| Control | 39.6 | | 38.7 | | 21.6 | |
| F4 50 g/hl | 31.7 | −19.9% | 44.5 | +15.0% | 23.7 | +9.7% |
| F4 200 | 32.3 | −18.5% | 44.8 | +16.0% | 22.9 | +5.7% |
| g/hl | | | | | | |
| C1 50 g/hl | 37.5 | −5.3% | 47.1 | +21.6% | 15.4 | −29.0% |
| C1 200 | 41.2 | +3.9% | 44.0 | +13.8% | 14.8 | −31.7% |
| g/hl | | | | | | |

It results clearly from Table 13 that a rearrangement in favor of the red polymers is obtained.

TABLE 14
Color intensity, shade and radiance of control and treated red
wines, after adding the products F4 and C1 with stirring, contact
time of 24 hours

| | Color | Varia- | | Varia- | | Varia- |
|---|---|---|---|---|---|---|
| | intensity | tion % | Shade | tion % | Radiance | tion % |
| Control | 1.0 | | 1.0 | | 30.4 | |
| F4 10 g/hl | 0.7 | −26% | 0.9 | −3.0% | 39.0 | +28% |
| F4 50 g/hl | 0.8 | −23% | 0.9 | −4.1% | 26.8 | −12% |
| F4 200 | 0.7 | −34% | 1.0 | +7.2% | 35.6 | +17% |
| g/hl | | | | | | |
| C1 10 g/hl | 0.9 | −15% | 1.0 | +1.0% | 32.9 | +8% |
| C1 50 g/hl | 0.8 | −25% | 0.9 | −4.1% | 35.1 | +15% |
| C1 200 | 0.8 | −18% | 1.1 | +10.3% | 29.2 | −4% |
| g/hl | | | | | | |

It results clearly from Table 14 that the color intensity decreases, and that the shade is stable, while the radiance is improved.

TABLE 15
Color composition of control and treated red wines, after adding the
products F4 and C1 with stirring, contact time of 24 hours: optical
density at absorption wavelengths 420, 520 and 620 nm

| | | | | Vari- | | Vari- |
|---|---|---|---|---|---|---|
| | OD | Variation | OD | ation | OD | ation |
| | 420 nm | % | 520 nm | % | 620 nm | % |
| Control | 40.7 | | 41.8 | | 17.9 | |
| F4 10 g/hl | 42.3 | +3.9% | 45.0 | +7.6% | 12.7 | −29.2% |
| F4 50 g/hl | 38.1 | −6.4% | 40.6 | −3.0% | 21.3 | +19.3% |
| F4 200 g/hl | 45.5 | +11.8% | 43.7 | +4.5% | 10.7 | −39.9% |
| C1 10 g/hl | 41.9 | +3.0% | 42.7 | +2.0% | 15.4 | −13.9% |
| C1 50 g/hl | 40.6 | −0.3% | 43.5 | +4.0% | 15.9 | −11.0% |
| C1 200 g/hl | 44.3 | +8.8% | 41.4 | −1.0% | 14.3 | −20.0% |

It results clearly from Table 15 that a very slight rearrangement of the color appears: the optical density at 420 nm (yellow-green) increases, the optical density at 520 nm (purple) increases and the optical density at 620 nm (blue-green) decreases.

### Example 13

### Treatment of Finished Wines with an Extract of Fungal Biomass, or an extract of Crustacean Biomass: White Wines

### Example 13a

### Treatment of White Wine without Stirring, with a Contact Time of 24 Hours

TABLE 16
Variations of pH and of content of total phenolic compounds in
control and treated white wines, after adding the products F4
and C1 without stirring, contact time of 24 hours

| | | Variation | TPC | Variation |
|---|---|---|---|---|
| | | (%) | (mg eq GAE/I) | (%) |
| Control | 3.65 | | 1000 | |
| F4 10 g/hl | 3.77 | +3.3% | 1000 | 0% |
| F4 50 g/hl | 3.77 | +3.3% | 975 | −2.5% |
| F4 200 g/hl | 3.79 | +3.8% | 975 | −2.5% |
| C1 10 g/hl | 3.79 | +3.8% | 1000 | 0% |
| C1 50 g/hl | 3.84 | +5.2% | 1000 | 0% |
| C1 200 g/hl | 3.97 | +8.8% | 910 | −9.0% |

It results clearly from Table 16 that the pH of the wine varies negligibly and that the polyphenols are conserved with a negligible variation.

TABLE 17
Color intensity, shade and tannin content of control and treated white
wines, after adding the products F4 and C1 without stirring, contact
time of 24 hours

| | | Vari- | | Vari- | | Vari- |
|---|---|---|---|---|---|---|
| | Color | ation | | ation | Tannins | ation |
| | intensity | % | Shade | % | (g/l) | % |
| Control | 0.58 | | 3.3 | | 1.23 | |
| F4 10 g/hl | 0.46 | −20.7% | 4.1 | +25.5% | 1.17 | −8.6% |
| F4 50 g/hl | 0.44 | −24.1% | 3.9 | +20.6% | 1.17 | −5.6% |
| F4 200 g/hl | 0.43 | −25.9% | 3.9 | +20.3% | 1.19 | −7.0% |
| C1 10 g/hl | 0.45 | −22.4% | 3.9 | +21.2% | 1.08 | −15.6% |
| C1 50 g/hl | 0.40 | −31.0% | 4.3 | +31.6% | 1.12 | −12.5% |
| C1 | 0.49 | −15.5% | 3.2 | −0.6% | 1.14 | −10.9% |
| 200 g/hl | | | | | | |

It results clearly from Table 17 that the color intensity decreases and the shade increases, while the total amount of tannins is conserved, with a negligible variation.

TABLE 18
Color composition of control and treated white wines, after adding
the products F4 and C1 without stirring, contact time of 24 hours:
optical density at absorption wavelengths 420, 520 and 620 nm

| | | | | Vari- | | Vari- |
|---|---|---|---|---|---|---|
| | OD | Variation | OD | ation | OD | ation |
| | 420 nm | % | 520 nm | % | 620 nm | % |
| Control | 70.7 | | 21.7 | | 7.6 | |
| F4 10 g/hl | 76.5 | +8.2% | 18.7 | −13.9% | 4.8 | −37.1% |
| F4 50 g/hl | 76.5 | +8.2% | 19.5 | −10.3% | 4.0 | −47.1% |
| F4 200 g/hl | 76.0 | +7.5% | 19.3 | −10.8% | 4.6 | −39.5% |
| C1 10 g/hl | 76.4 | +8.1% | 19.3 | −10.9% | 4.2 | −44.5% |
| C1 50 g/hl | 78.1 | +10.5% | 18.2 | −16.2% | 3.7 | −51.6% |
| C1 200 g/hl | 70.9 | +0.2% | 21.9 | +0.7% | 7.3 | −4.2% |

It results clearly from Table 18 that a very slight rearrangement of the color appears: the optical density at 420 nm (yellow-green) increases and the optical density at 520 nm (purple) and at 620 nm (blue-green) decrease.

### Example 13b

### Treatment of White Wine with Gentle Stirring, with a Contact Time of 24 Hours

TABLE 19
Variations of pH and of content of total phenolic compounds in
control and treated white wines, after adding the products F4
and C1 with stirring, contact time of 24 hours

| | | Variation | TPC | Variation |
|---|---|---|---|---|
| | | (%) | (mg eq GAE/I) | (%) |
| Control | 3.73 | | 1000 | |
| F4 10 g/hl | 3.76 | +0.8% | 909 | −9.1% |
| F4 50 g/hl | 3.77 | +1.0% | 909 | −9.1% |
| F4 200 g/hl | 3.81 | +2.1% | 873 | −12.7% |
| C1 10 g/hl | 3.77 | +1.1% | 873 | −12.7% |
| C1 50 g/hl | 3.80 | +1.9% | 862 | −13.8% |
| C1 200 g/hl | 3.98 | +6.7% | 850 | −15.0% |

It results clearly from Table 19 that the pH of the wine varies negligibly and that the total amount of polyphenols is conserved, with a negligible variation.

TABLE 20
Color intensity, shade and tannin content of control and treated white
wines, after adding the products F4 and C1 with stirring, contact time
of 24 hours

| | | Vari- | | Vari- | | Vari- |
|---|---|---|---|---|---|---|
| | Color | ation | | ation | Tannins | ation |
| | intensity | % | Shade | % | (g/l) | % |
| Control | 0.54 | | 3.4 | | 1.28 | |
| F4 10 g/hl | 0.56 | +3.7% | 3.3 | −3.5% | 0.94 | −26.6% |
| F4 50 g/hl | 0.53 | −1.9% | 3.2 | −5.0% | 0.90 | −29.7% |
| F4 200 g/hl | 0.49 | −9.3% | 3.3 | −2.9% | 0.96 | −25.0% |
| C1 10 g/hl | 0.52 | −3.7% | 3.2 | −5.9% | 1.00 | −21.9% |
| C1 50 g/hl | 0.51 | −5.6% | 3.2 | −6.8% | 0.81 | −36.7% |
| C1 200 g/hl | 1.09 | +102% | 2.2 | −35.3% | 0.77 | −39.8% |

It results clearly from Table 20 that the color intensity decreases and the shade decreases, while the total amount of tannins decreases slightly.

TABLE 21
Color composition of control and treated white wines, after adding
the products F4 and C1 with stirring, contact time of 24 hours:
optical density at absorption wavelengths 420, 520 and 620 nm

| | OD | Variation | OD | Variation | OD | Varia- |
|---|---|---|---|---|---|---|
| | 420 nm | % | 520 nm | % | 620 nm | tion % |
| Control | 71.8 | | 21.1 | | 7.1 | |
| F4 10 g/hl | 70.4 | −1.9% | 21.4 | +1.4% | 8.1 | +14.7% |
| F4 50 g/hl | 70.4 | −1.9% | 21.7 | +2.9% | 7.8 | +9.9% |
| F4 | 71.8 | +0.1% | 21.7 | +2.9% | 6.4 | −9.4% |
| 200 g/hl | | | | | | |
| C1 10 g/hl | 72.0 | +0.3% | 22.5 | +6.4% | 5.5 | −22.3% |
| C1 50 g/hl | 69.8 | −2.8% | 22.0 | +4.0% | 6.2 | +15.9% |
| C1 | 59.2 | −17.5% | 26.9 | +27.2% | 13.9 | +96.2% |
| 200 g/hl | | | | | | |

It results clearly from Table 21 that a very slight rearrangement of the color appears: the optical density at 420 nm (yellow-green) decreases and the optical density at 520 nm (purple) and at 620 nm (blue-green) increase.

### Example 14

### Treatment of Finished Wines with an Extract of Fungal Biomass or an Extract of Crustacean Biomass: Rosé Wines

### Example 4a

### Treatment of Rose Wine without Stirring, with a Contact Time of 24 Hours

TABLE 22
Variations of pH and of content of total phenolic compounds in
control and treated rosé wines, after adding the products F4
and C1 without stirring, contact time of 24 hours

| | | Variation | TPC | Variation |
|---|---|---|---|---|
| | | (%) | (mg eq GAE/I) | (%) |
| Control | 3.55 | | 365 | |
| F4 10 g/hl | 3.55 | +0.0% | 347 | −4.9% |
| F4 50 g/hl | 3.56 | +0.3% | 365 | +0.0% |
| F4 200 g/hl | 3.60 | +1.4% | 350 | −4.1% |
| C1 10 g/hl | 3.55 | +0.0% | 342 | −6.3% |
| C1 50 g/hl | 3.56 | +0.3% | 350 | −4.1% |
| C1 200 g/hl | 3.60 | +1.4% | 325 | −11.0% |

It results clearly from Table 22 that the pH of the wine varies negligibly and the total amount of polyphenols is conserved, with a negligible variation.

TABLE 23
Fractionation of the phenolic material of control and treated rosé
wines, after adding the products F4 and C1 without stirring,
contact time of 24 hours

| | | | Red | | Brown | |
|---|---|---|---|---|---|---|
| | Monomers | Variation | polymers | Variation | polymers | Variation |
| | % | % | % | % | % | % |
| Control | 52.5 | | 43.6 | | 4.0 | |
| F4 50 g/hl | 58.7 | +11.8% | 36.4 | −16.5% | 5.0 | +25.3% |
| F4 200 g/hl | 27.3 | +9.1% | 38.5 | −11.7% | 4.3 | +7.8% |
| C1 50 g/hl | 44.4 | −15.4% | 40.2 | −7.8% | 15.4 | +288% |
| C1 200 g/hl | 45.7 | −12.8% | 41.1 | −5.7% | 13.2 | +232% |

It results clearly from Table 23 that a rearrangement in favor of the brown monomers and polymers is obtained.

TABLE 24
Color intensity, shade and radiance of control and treated rosé wines,
after adding the products F4 and C1. without stirring, contact time of
24 hours

| | | Vari- | | Vari- | | Vari- |
|---|---|---|---|---|---|---|
| | Color | ation | | ation | Radi- | ation |
| | intensity | % | Shade | % | ance | % |
| Control | 0.13 | | 1.0 | | 40.8 | |
| F4 10 g/hl | 0.14 | +7.7% | 1.0 | +1.0% | 39.4 | −3.6% |
| F4 50 g/hl | 0.15 | +15.4% | 1.1 | +3.9% | 35.8 | −12.3% |
| F4 200 g/hl | 0.12 | −7.7% | 1.0 | +1.0% | 40.5 | −0.7% |
| C1 10 g/hl | 0.13 | +0.0% | 1.1 | +1.9% | 40.1 | −1.7% |
| C1 50 g/hl | 0.12 | −7.7% | 1.0 | +1.0% | 41.2 | +1.0% |
| C1 | 0.37 | +185% | 1.3 | +26.2% | 13.1 | −67.9% |
| 200 g/hl | | | | | | |

It results clearly from Table 24 that the color intensity decreases, while the shade and the radiance are stable.

TABLE 25
Color composition of control and treated rosé wines, after adding the
products F4 and C1 without stirring, contact time of 24 hours

| | | Vari- | | Vari- | | Vari- |
|---|---|---|---|---|---|---|
| | OD | ation | OD | ation | OD | ation |
| | 420 nm | % | 520 nm | % | 620 nm | % |
| Control | 52.5 | | 43.5 | | 3.9 | |
| F4 10 g/hl | 47.1 | −10.3% | 45.1 | +3.7% | 7.7 | +97% |
| F4 50 g/hl | 46.7 | −11.1% | 43.7 | +0.5% | 9.5 | +144% |
| F4 200 g/hl | 47.7 | −9.2% | 45.6 | +4.8% | 6.7 | +70.5% |
| C1 10 g/hl | 47.9 | −8.8% | 45.5 | +4.6% | 6.5 | +67% |
| C1 50 g/hl | 48.1 | −8.4% | 45.9 | +5.5% | 6.0 | +54% |
| C1 200 g/hl | 47.7 | −9.1% | 36.5 | −16.1% | 15.7 | +303% |

It results clearly from Table 25 that a very slight rearrangement of the color appears: the optical density at 420 nm (yellow-green) decreases, and the optical density at 520 nm (purple) and at 620 nm (blue-green) increase.

### Example 14b

### Treatment of Rosé Wine with Gentle Stirring, with a Contact Time of 24 Hours

TABLE 26
Variations of pH and of content of total phenolic compounds in
control and treated rosé wines, after adding the products F4
and C1 with stirring, contact time of 24 hours

| | | Variation | TPC | Variation |
|---|---|---|---|---|
| | | (%) | (mg eq GAE/I) | (%) |
| Control | 3.54 | | 365 | |
| F4 10 g/hl | 3.51 | −0.9% | 255 | −30.0% |
| F4 50 g/hl | 3.50 | −1.1% | 269 | −26.3% |
| F4 200 g/hl | 3.56 | +0.6% | 237 | −34.9% |
| C1 10 g/hl | 3.55 | +0.3% | 309 | −15.2% |
| C1 50 g/hl | 3.59 | +1.4% | 324 | −11.1% |
| C1 200 g/hl | 3.71 | +4.8% | 279 | −23.3% |

It results clearly from Table 26 that the pH of the wine varies negligibly and the total amount of polyphenols is conserved, with a negligible variation.

TABLE 27
Fractionation of the phenolic material of control and treated rosé
wines, after adding the products F4 and C1 with stirring,
contact time of 24 hours

| | Monomers | Variation | Red polymers | Variation |
|---|---|---|---|---|
| | % | % | % | % |
| Control | 61.1 | | 38.9 | |
| F4 50 g/hl | 57.1 | −6.4% | 42.9 | +10.1% |
| F4 200 g/hl | 57.5 | −5.9% | 42.1 | 8.0% |
| C1 50 g/hl | 63.6 | +4.1% | 36.4 | −6.4% |
| C1 200 g/hl | 65.9 | +7.9% | 34.1 | −12.4% |

It results clearly from Table 27 that a rearrangement in favor of the red polymers is obtained.

TABLE 28
Color intensity, shade and radiance of control and treated rosé wines,
after adding the products F4 and C1 with stirring, contact time of
24 hours

| | | Vari- | | Vari- | | Vari- |
|---|---|---|---|---|---|---|
| | Color | ation | | ation | | ation |
| | intensity | % | Shade | % | Radiance | % |
| Control | 0.12 | | 0.99 | | 99.9 | |
| F4 10 g/hl | 0.14 | +16.7% | 1.02 | +3.0% | 99.9 | −0.0 |
| F4 50 g/hl | 0.14 | +16.7% | 1.01 | +2.0% | 99.9 | −0.0 |
| F4 200 g/hl | 0.12 | +0.0% | 1.00 | +1.0% | 99.9 | −0.0 |
| C1 10 g/hl | 0.13 | +8.3% | 1.03 | +4.0% | 99.9 | −0.0 |
| C1 50 g/hl | 0.11 | −8.3% | 1.01 | +2.0% | 99.9 | +0.0 |
| C1 200 g/hl | 0.16 | +33.3% | 1.10 | +11.1% | 99.9 | −0.0 |

It results clearly from Table 28 that the color intensity increases and that the shade and the radiance are stable.

TABLE 29
Color composition of control and treated rosé wines, after adding the
products F4 and C1 with stirring, contact time of 24 hours

| | | | | Vari- | | Vari- |
|---|---|---|---|---|---|---|
| | OD | Variation | OD | ation | OD | ation |
| | 420 nm | % | 520 nm | % | 620 nm | % |
| Control | 46.6 | | 47.2 | | +6.2 | |
| F4 10 g/hl | 46.0 | −1.2% | 44.7 | −5.3% | +9.3 | +49.5% |
| F4 50 g/hl | 45.9 | −1.3% | 45.2 | −4.2% | +8.8 | +42.2% |
| F4 200 g/hl | 46.7 | +0.3% | 46.3 | −2.0% | +7.0 | +12.6% |
| C1 10 g/hl | 46.9 | +0.7% | 45.4 | −3.8% | +7.7 | +23.4% |
| C1 50 g/hl | 46.6 | +0.2% | 46.1 | −2.3% | +7.2 | +16.6% |
| C1 200 g/hl | 46.8 | +0.5% | 42.2 | −10.6% | +11.0 | +76.8% |

It results clearly from Table 29 that a very slight rearrangement of the color appears: the optical density at 420 nm (yellow-green) is stable, the optical density at 520 nm (purple) decreases and the optical density at 620 nm (blue-green) increases.

In Examples 15 and 16, the content of heavy metals and major metals was determined by atomic absorption spectrometry.

For these studies, the following were used:

a red vin de pays wine of merlot grape variety, vintage 2003, from the La Lande Pennautier vineyard (Aude). The grape underwent maceration but was not clarified or filtered. Its content of total phenolic compounds is 2075 mg GAE/l. This wine is conditioned in 750 ml bottles;

a white vin de pays wine of chardonnary grape variety, vintage 2003, from the La Lande Pennautier vineyard (Aude). The grape underwent direct pressing followed by vinification at low temperature at 20° C. Its content of total phenolic compounds is 273.3 mg GAE/l. This wine is conditioned in 750 ml bottles;

a natural sweet wine of grenache and macabeu grape varieties, vintage 2003, from the Baixas cooperative winery (Pyrénées Orientales). The grape underwent direct pressing, clarification using ground bentonite and then mutage with 96 vol % pure alcohol on must and finally deproteinating clarification and centrifugation. Its content of total phenolic compounds is 370.8 mg GAE/l. This wine is conditioned in 750 ml bottles.

The assay method used to analyze the removal of the heavy metals (lead, cadmium) and major metals (iron) in Examples 15 and 16 is: “Determination of the mineral content of various tests” (according to the official method of the OIV: Recueil des méthodes internationales d'analyses du vin et des moûts [Collection of the international methods for the analysis of wine and musts] p 217-224, p 227-228, p 231-234). The copper and iron contents were determined by flame atomic absorption spectrometry (AAS). The cadmium and lead contents were determined by oven atomic absorption spectrometry (AAS).

### Example 15

### Removal of the Heavy Metals (Lead, Cadmium) in Red, White and Natural Sweet Wines

Red, white and natural sweet wines were artificially contaminated with the heavy metals lead to 500 μg/l and cadmium to 20 μg/l (simultaneously). The extracts F1, F2, F7 or C1 are placed in contact with the wines at doses of 10, 50 or 200 g/hl. The contents of metals in the control wines and in the treated wines are determined by graphite oven atomic absorption spectrometry.

As a reminder, the OIV recommendations regarding the maximum content of heavy metals in wines are 200 μg/l for lead and 10 μg/l for cadmium.

TABLE 30
Removal of heavy metals (lead and cadmium) in red, white and sweet
wines

| | | Lead (μg/l) | | | Cadmium (μg/l) | | |
|---|---|---|---|---|---|---|---|
| Initial content | | Red | White | Sweet | Red | White | Sweet |
| (μg/l) | | 150 | 111 | 110 | 19 | 18 | 10 |
| F1 | 200 g/hl | 33% | 58% | 38% | 54% | 17% | 25% |
| F1 | 50 g/hl | 31% | 29% | 26% | 56% | 12% | 12% |
| F1 | 10 g/hl | 21% | 10% | 32% | 57% | 18% | 13% |
| F2 | 200 g/hl | 51% | 50% | — | 21% | 17% | 17% |
| F2 | 50 g/hl | 41% | 44% | 15% | 27% | 23% | 17% |
| F2 | 10 g/h/ | 41% | 27% | 42% | 14% | 19% | 21% |
| F7 | 200 g/hl | 74% | 65% | 84% | 25% | 8% | 17% |
| F7 | 50 g/hl | 66% | 52% | 54% | 29% | 5% | 23% |
| F7 | 10 g/hl | 37% | 43% | 47% | 26% | 11% | 6% |
| C1 | 200 g/hl | 40% | 73% | 88% | 32% | 38% | 17% |
| C1 | 50 g/hl | 32% | 78% | 78% | 21% | 38% | 43% |
| C1 | 10 g/hl | 17% | 50% | 0% | 22% | 38% | 19% |

It results clearly from Table 30 that the lead and cadmium are removed to an amount of 50% for lead and 57% for cadmium.

### Example 16

### Use of the Fungal Extracts According to the Present Invention to Prevent Breakage Due to the Presence of Iron in Red, White and Sweet Wines

Red, white and natural sweet wines were artificially contaminated with iron to 20 mg/l. The extracts F1, F2, F7 or C1 are placed in contact with the wines at doses of 10, 50 or 200 g/hl. The iron content in the control wines and in the treated wines are determined by flame atomic absorption spectrometry.

TABLE 31
Removal of iron in red, white and sweet wines

| | Red wine | White wine | Sweet wine |
|---|---|---|---|
| Initial iron | 23 | 6 | 5 |
| content (mg/l) | | | |

| | | | | |
|---|---|---|---|---|
| F1 | 200 g/hl | 73% | 32% | 77% |
| F1 | 50 g/hl | 72% | 22% | 42% |
| F1 | 10 g/hl | 70% | 20% | 23% |
| F2 | 200 g/hl | 80% | 34% | 51% |
| F2 | 50 g/hl | 72% | 16% | 24% |
| F2 | 10 g/hl | 71% | 24% | 10% |
| F7 | 200 g/hl | 90% | 91% | 98% |
| F7 | 50 g/hl | 86% | 54% | 90% |
| F7 | 10 g/hl | 75% | 20% | 59% |
| C1 | 200 g/hl | 91% | 80% | 94% |
| C1 | 50 g/hl | 77% | 60% | 88% |
| C1 | 10 g/hl | 73% | 25% | 59% |

It results clearly from Table 31 that the iron is removed up to 80%.

### Example 17

### Removal of Mycotoxins in Red, White and Natural Sweet Wines

For this study, red, white and sweet wines identical to those of Examples 15 and 16 were used. These red, white and natural sweet wines were artificially contaminated with ochratoxin A (OTA) at a dose of 5 μg/l. The extracts F1, F2, F7 or C1 are placed in contact with the wines at doses of 500 g/hl. As a reminder, the OIV recommends not exceeding an ochratoxin A content of 2 μg/l in wines. No specific treatment has been acknowledged to date.

The ochratoxin A contents in the control wines and in the treated wines are determined by the official method of the OIV (Oeno resolution 16/2001). The assay is performed by calculating the OTA content by assaying the ochratoxin A in the wine after passage through an immunoaffinity column and HPLC with fluorimetric detection, according to “Determination of ochratoxin A in wine by means of immunoaffinity column clean-up and high-performance liquid chromatography.” A. Visconti, M. Pascale, G. Centonze. Journal of Chromatography A, 864 (1999) 89-101.

TABLE 32
Removal of mycotoxins in red, white and natural sweet wines, at
various pH values

| | Red wine | | | White wine | | | Natural sweet wine | | |
|---|---|---|---|---|---|---|---|---|---|
| | | OTA | | | OTA | | | OTA | |
| | pH | (μg/l) | % | | (μg/l) | % | pH | (μg/l) | % |
| Control | | 3.0 | | | 4.5 | | | 4.6 | |
| F1 | 3.11 | 1.4 | 53% | 3.08 | 2.0 | 56% | 3.14 | 2.5 | 46% |
| | 4.09 | 1.3 | 57% | 3.78 | 1.6 | 65% | 4.04 | 2.6 | 43% |
| | 4.39 | 1.5 | 50% | 4.25 | 2.5 | 45% | 4.39 | 2.5 | 46% |
| F2 | 3.08 | 0.8 | 73% | 3.07 | 1.4 | 69% | 3.09 | 2.3 | 50% |
| | 4.10 | 1.0 | 67% | 3.79 | 2.1 | 53% | 4.03 | 3.0 | 35% |
| | 4.39 | 1.1 | 63% | 4.34 | 2.2 | 51% | 4.35 | 2.3 | 50% |
| F7 | 3.51 | 1.0 | 66% | 3.54 | 2.6 | 42% | 3.61 | 3.8 | 17% |
| | 4.52 | 0.5 | 83% | 4.22 | 2.1 | 53% | 4.61 | 3.4 | 26% |
| | 4.82 | 0.6 | 80% | 4.72 | 1.9 | 58% | 4.92 | 3.5 | 24% |
| C1 | 3.55 | 0.9 | 70% | 3.45 | 3.8 | 16% | 3.70 | 4.2 | 9% |
| | 4.55 | 1.1 | 63% | 4.20 | 3.3 | 27% | 4.62 | 2.7 | 41% |
| | 4.70 | 0.9 | 70% | 4.58 | 4.0 | 11% | 4.90 | 4.3 | 7% |

It results clearly from Table 32 that ochratoxin A is removed up to 73%. The amount of mycotoxins is then below the recommendations for red and white wines.

### Example 18

### Clarification of Red Wine Musts with F1 at a Dose of 50 g/hl, Relative to a Control that has Undergone a Natural Decantation (10 l Tank)

A must from tank A (10 l tank) and a must from tank B (10 l tank) were treated by adding fungal extract F1. This extract is added at the end of the alcoholic fermentation at a dose of 50 g/hl. The control must is clarified by natural decantation.

The standard analyses of sugar, TAV, total acidity (T Ac.), volatile acidity (V Ac.), total SO₂ (T SO₂), volatile SO₂ (L SO₂), the pH and the turbidity are performed.

Results:

TABLE 33
Variation of the turbidity of musts and red wines obtained from the
must of tank A (10 l tank)

| | Turbidity (NTU) | Variation (%) |
|---|---|---|
| Control starting must | 1655 | |
| Control wine | 1655 | 0.0% |
| Treated wine - F1 50 g/l | 25 | 98.5% |

TABLE 34
Analytical characteristics of the musts and red wines obtained from
the must of tank A (10 l tank)

| | Sugar | TAV | T Ac. | V Ac. | T SO₂ | L SO₂ | |
|---|---|---|---|---|---|---|---|
| | (g/l) | (% vol) | (g/l H₂SO₄) | (g/l H₂SO₄) | (mg/l) | (mg/l) | pH |
| Starting must | 198.0 | 0.05 | 2.82 | 0.05 | 18 | 3 | 3.43 |
| Control wine | 3.1 | 13.23 | 4.44 | 0.27 | 30 | 2 | 3.24 |
| Treated wine - | 3.6 | 13.24 | 4.50 | 0.24 | 28 | 2 | 3.25 |
| F1 50 g/hl | | | | | | | |

TABLE 35
Variation of the turbidity of musts and of red wines obtained
from the must of tank B (10 l tank)

| | Turbidity (NTU) | Variation (%) |
|---|---|---|
| Control starting must | 3048 | 5 |
| Control wine | 2332 | 23.4% |
| Treated wine - F1 50 g/hl | 749 | 75.4% |

TABLE 36
Analytical characteristics of the musts and red wines obtained from
the must of tank B (10 l tank)

| | Sugar | TAV | T Ac. | V Ac. | T SO₂ | L SO₂ | |
|---|---|---|---|---|---|---|---|
| | (g/l) | (% vol) | (g/l H₂SO₄) | (g/l H₂SO₄) | (mg/l) | (mg/l) | pH |
| Control starting | 195.0 | 0.05 | 2.82 | 0.05 | 18 | 3 | 3.43 |
| must | | | | | | | |
| Control wine | 4.0 | 12.86 | 3.99 | 0.34 | 47 | 2 | 3.39 |
| Treated wine - F1 | 3.9 | 12.89 | 4.09 | 0.37 | 51 | 2 | 3.43 |
| 50 g/hl | | | | | | | |

Treatment with chitin-glucan F1 contributes towards improving the clarification of the red wines, without impairing the content of total phenolic compounds and tannins, compared with the control wine that has undergone a natural decantation.

### Example 19

### Clarification of Natural Sweet Wine Musts in Poor Health State with F1 at a Dose of 50 g/hl, Compared with a Control that has Undergone a Traditional Clarification (Gelatin/Bentonite)

A natural sweet wine must in poor health state from vineyard D (3 hl tank) was treated by adding fungal extract F1 before sludge removal (before alcoholic fermentation), at a dose of 50 g/hl. The control wine underwent clarification with the traditional products, gelatin and bentonite.

Results:

TABLE 37
Variation of the turbidity (3 hl tank, poor health state)

| | Turbidity (NTU) | Variation (%) |
|---|---|---|
| Control starting must | 333.5 | |
| Control wine | 1.76 | −99.5% |
| Treated wine - F1 | 1.66 | −99.5% |
| 50 g/hl | | |

TABLE 38
Analytical characteristics of musts and of natural sweet wines
obtained from the must from vineyard D (3 hl tank, poor health state)

| | Sugar | TAV | T Ac. | V Ac. | T SO₂ | L SO₂ | |
|---|---|---|---|---|---|---|---|
| | (g/l) | (% vol) | (g/l H₂SO₄) | (g/l H₂SO₄) | (mg/l) | (mg/l) | pH |
| Control starting | 267.6 | 0.01 | 1.34 | 0.0 | 13 | 3 | 3.47 |
| must | | | | | | | |
| Control wine | 106.0 | 14.86 | 2.76 | 0.43 | 1151 | 400 | 3.75 |
| Treated | 94.0 | 16.86 | 2.98 | 0.53 | 140 | 50 | 3.74 |
| wine - F1 | | | | | | | |
| 50 g/hl | | | | | | | |

TABLE 39
Total phenolic compounds (TPC), tannins and color intensity
of musts and of natural sweet wines obtained from the must
from vineyard D (3 hl tank, poor health state)

| | TPC | | |
|---|---|---|---|
| | (mg eq | Tannins | |
| | gallic acid/l) | (g/l) | OD 280 |
| Control starting must | 1017.2 | 0.28 | 0.1 |
| Control wine | 665.3 | 0.20 | 0.1 |
| Treated wine - F1 50 g/hl | 753.5 | 0.20 | 0.1 |

The addition of F1 gives rise to a decrease in the turbidity equivalent to that obtained after traditional treatment (gelatin/bentonite), without impairing the content of total phenolic compounds and of tannins, or the color intensity.

### Example 20

### Clarification of Natural Sweet Wine Musts in Good Health Stage with F1 at a Dose of 50 g/hl Compared with a Control that has Undergone a Traditional Clarification (Gelatin/Bentonite)

A natural sweet wine must in good health state from vineyard D (3 hl tank) was treated by adding fungal extract F1 before sludge removal (before alcoholic fermentation), at a dose of 50 g/hl. The control wine underwent clarification with the traditional products gelatin and bentonite.

2—Results:

TABLE 40
Variation of the turbidity (3 hl tank, good health state)

| | Turbidity (NTU) | Variation (%) |
|---|---|---|
| Control starting must | 147.0 | |
| Control wine | 2.5 | −98.3% |
| Wine treated by sludge removal - F1 | 3.9 | −97.3% |
| 50 g/hl | | |
| Wine treated before mutage - 50 g/hl | 3.6 | −97.5% |

TABLE 41
Analytical characteristics of musts and of natural sweet wines
obtained from the must from vineyard D (3 hl tank, good health state)

| | Sugar | TAV | T Ac. | V Ac. | T SO₂ | L SO₂ | |
|---|---|---|---|---|---|---|---|
| | (g/l) | (% vol) | (g/l H₂SO₄) | (g/l H₂SO₄) | (mg/l) | (mg/l) | pH |
| Control | 212.0 | 0.01 | 2.09 | 0.0 | 18 | 3 | 3.44 |
| starting must | | | | | | | |
| Control wine | 120.0 | 16.86 | 2.82 | 0.64 | 86 | 2 | 3.81 |
| Wine treated | 102.0 | 18.37 | 2.75 | 0.58 | 60 | 2 | 3.82 |
| by sludge | | | | | | | |
| removal - F1 | | | | | | | |
| 50 g/hl | | | | | | | |
| Wine treated | 115.0 | 17.54 | 2.77 | 0.62 | 64 | 2 | 3.81 |
| before mutage - | | | | | | | |
| F1 50 g/hl | | | | | | | |

TABLE 42
Total phenolic compounds (TPC), tannins and color intensity
of musts and of natural sweet wines obtained from the must
from vineyard D (3 hl tank, good health state)

| | TPC | | |
|---|---|---|---|
| | (mg eq | Tannins | OD |
| | gallic acid/l) | (g/l) | 280 |
| Control starting must | 790.6 | 0.09 | 0.09 |
| Control wine | 291.3 | 0.05 | 0.07 |
| Wine treated by sludge removal | 280.0 | 0.06 | 0.07 |
| F1 50 g/hl | | | |
| Wine treated before mutage - F1 | 289.0 | 0.06 | 0.07 |
| 50 g/hl | | | |

The addition of F1 gives rise to a decrease in turbidity equivalent to that obtained after traditional treatment (gelatin/bentonite), without impairing the content of total phenolic compounds and of tannins, or the color intensity. Furthermore, the addition of F1 on sludge removal or before mutage has no effect on the quality of the clarification.

### Example 21

### Clarification of Rosé Wine Musts with F1 at a Dose of 50 g/hl, Compared with a Control that has Undergone a Traditional Clarification

A rosé wine must from tank C (300 hl tank) was treated by adding fungal extract F1 after alcoholic fermentation, at a dose of 50 g/hl. The control underwent a traditional clarification.

Results:

TABLE 43
Variation of the turbidity (300 hl tank)

| | Turbidity (NTU) |
|---|---|
| Control tank must | 1710 |
| Control wine | 185 |
| Treated wine - F1 50 g/hl | 82 |

TABLE 44
Analytical characteristics of the musts and of the rose wines obtained
from the must from tank C (300 hl tank)

| | Sugar | TAV | T Ac. | V Ac. | T SO₂ | L SO₂ | |
|---|---|---|---|---|---|---|---|
| | (g/l) | (% vol) | (g/l H₂SO₄) | (g/l H₂SO₄) | (mg/l) | (mg/l) | pH |
| Control starting | 100 | 0.01 | 1.71 | 0.0 | 11 | 2 | 3.45 |
| must | | | | | | | |
| Control wine | 1.9 | 13.78 | 2.98 | 0.26 | 64 | 2 | 3.55 |
| Treated must - | 110 | 0.01 | 1.83 | 0.0 | 14 | 2 | 3.44 |
| F1 50 g/hl | | | | | | | |
| Treated wine - F1 | 1.7 | 13.37 | 3.01 | 0.23 | 56 | 5 | 3.56 |
| 50 g/hl | | | | | | | |

Treatment of the rosé wine must after alcoholic fermentation with F1 allows the rosé wine musts to be clarified just as efficiently as by traditional clarification. The content of total phenolic compounds, the anthocyan content and the optical density at 280 nm remain unchanged relative to the control.

### Example 22

### Removal of Mycotoxins in Naturally Contaminated Red Wines

A red wine from vineyard C (426 hl tank) and a red wine from vineyard D (3 hl tanks) containing ochratoxin A contents close to the maximum content recommended by the OIV (2 μg/l) were treated by adding fungal extract F1. Several treatment assays were tested: 129 g/hl, 300 g/hl, 400 g/hl and 500 g/hl. The fungal extract is left in contact with the wine for 3 days. The control wine undergoes no treatment.

Results:

TABLE 45
Removal of mycotoxins in the red wine C (426 hl tank)

| | OTA (μg/l) | Variation (%) |
|---|---|---|
| Control | 1.7 | — |
| F1 - 129 g/hl | 1.4 | −17.6% |

TABLE 46
Analytical characteristics of the red wine C (426 hl tank)

| | | | T Ac. | V Ac. | | | |
|---|---|---|---|---|---|---|---|
| | Sugar | TAV | (g/l | (g/l | T SO₂ | L SO₂ | |
| | (g/l) | (% vol) | H₂SO₄) | H₂SO₄) | (mg/l) | (mg/l) | pH |
| Control | 1.9 | 12.50 | 10.28 | 7.82 | 28 | 3 | 3.38 |
| F1 - | 2.1 | 12.57 | 9.69 | 7.24 | 28 | 3 | 3.37 |
| 129 g/hl | | | | | | | |

The treatment F1 at a dose of 129 g/hl gives rise to a 17.6% reduction in the OTA content of the red wine. The treatment F1 does not result in any change of the standard analytical parameters of the wines. The contents of total phenolic compounds (˜2400 mg/l), of tannins (˜3.3 g/l) and of anthocyans (˜470 mg/l) and the color intensity (OD 280 nm=0.57) on samples taken 2 days and 1 week after treatment are unchanged relative to the control.

TABLE 47
Removal of the mycotoxins in the red wine from vineyard D (3 hl
tank)

| | OTA (μg/l) | Variation (%) |
|---|---|---|
| Control | 2.7 | — |
| F1 - 300 g/hl | 2.2 | −18.5% |
| F1 - 400 g/hl | 2.1 | −22.2% |
| F1 - 500 g/hl | 2.0 | −25.9% |

TABLE 48
Analytical characteristics of the red wine from vineyard D (3 hl tank)

| | | | T Ac. | V Ac. | | | |
|---|---|---|---|---|---|---|---|
| | Sugar | TAV | (g/l | (g/l | T SO₂ | L SO₂ | |
| | (g/l) | (% vol) | H₂SO₄) | H₂SO₄) | (mg/l) | (mg/l) | pH |
| Control | 1.5 | 13.66 | 3.47 | 0.59 | 25 | 2 | 3.55 |
| F1 - | 1.4 | 13.63 | 3.42 | 0.59 | 30 | 3 | 3.55 |
| 300 g/hl | | | | | | | |
| F1 - | 1.5 | 13.51 | 4.78 | 2.30 | 34 | 2 | 3.52 |
| 400 g/hl | | | | | | | |
| F1 - | 1.4 | 13.33 | 6.61 | 4.19 | 34 | 2 | 3.43 |
| 500 g/hl | | | | | | | |

The removal of the OTA in the red wine is dose dependent. The contents of total phenolic compounds (˜2070 mg/l), of tannins (˜2.43 g/l) and the color intensity (OD 280 nm=0.47) after treatment are unchanged relative to the control. Irrespective of the dose of treatment F1 added to the wine (300 g/hl, 400 g/hl, 500 g/hl), this does not give rise to any changes in the standard analytical parameters of the wines.

### Example 23

### Small-Scale Removal of the Mycotoxins in Naturally Contaminated Red Wines

A red wine from vineyard M (1 l bottles) containing OTA contents greater than or equal to the OIV recommendation were treated by adding fungal extract F1, at several doses and under variable temperature and time conditions, in one or two additions. The fungal extract is left in contact with the wine for 3 days. The control wine undergoes no treatment.

Results:

TABLE 49
Analytical characteristics of the red wines from vineyard M
(1 l bottles)

| | Sugar | TAV | T Ac. | V Ac. | T SO₂ | L SO₂ | |
|---|---|---|---|---|---|---|---|
| | (g/l) | (% vol) | (g/l H₂SO₄) | (g/l H₂SO₄) | (mg/l) | (mg/l) | pH |
| Control | 1.1 | 12.97 | 5.36 | 2.60 | 13 | 2 | 3.69 |
| F1 - 200 g/hl | 1.2 | 13.09 | 3.65 | 0.56 | 6 | 2 | 3.73 |
| Room | | | | | | | |
| temperature, | | | | | | | |
| 3 days | | | | | | | |
| F1 - 300 g/hl | 1.1 | 13.14 | 3.64 | 0.55 | 6 | 2 | 3.73 |
| 0° C., 3 days | | | | | | | |
| F1 - 400 g/hl | 1.3 | 13.03 | 3.61 | 0.54 | 7 | 2 | 3.74 |
| Room | | | | | | | |
| temperature, | | | | | | | |
| 3 days | | | | | | | |
| F1 - 2 × 200 g/hl | 1.3 | 13.05 | 3.59 | 0.57 | 5 | 2 | 3.76 |
| Room | | | | | | | |
| temperature, | | | | | | | |
| 3 days | | | | | | | |
| F1 - 500 g/hl | 1.1 | 13.10 | 3.59 | 0.55 | 6 | 2 | 3.74 |
| Room | | | | | | | |
| temperature, | | | | | | | |
| 3 days | | | | | | | |
| F1 - 500 g/hl | 1.0 | 13.11 | 3.59 | 0.56 | 7 | 2 | 3.75 |
| 0° C., 3 days | | | | | | | |
| F1 - 300 g/hl | 1.0 | 12.89 | 3.62 | 0.59 | 7 | 2 | 3.73 |
| 10 days | | | | | | | |

TABLE 50
Removal of the mycotoxins in the red wine from vineyard M (1 l
bottles)

| | OTA (μg/l) | Variation (%) |
|---|---|---|
| Control | 3.0 | |
| F1 - 200 g/hl | 2.3 | −23.3% |
| Room temperature, | | |
| 3 days | | |
| F1 - 300 g/hl | 2.0 | −33.3% |
| 0° C., 3 days | | |
| F1 - 400 g/hl | 1.9 | −36.7% |
| Room temperature, | | |
| 3 days | | |
| F1 - 2 × 200 g/hl | 1.8 | −40% |
| Room temperature, | | |
| 3 days | | |
| F1 - 500 g/hl | 2.0 | −33.3% |
| Room temperature, | | |
| 3 days | | |
| F1 - 500 g/hl | 1.9 | −36.7% |
| 0° C., 3 days | | |
| F1 - 300 g/hl | 2.0 | −33.3% |
| 10 days | | |

The removal is at least 23% with the treatment F1 at a dose of 200 g/hl. The contents of total phenolic compounds (˜2432 mg/l), of tannins (˜2.95 g/l) and of anthocyans (˜415 mg/l) and the color intensity (OD 280 nm=0.56) after treatment are unchanged relative to the control.

The most efficient treatment protocol is the successive addition of 2 times 200 g/hl of F1, which makes it possible to reduce the OTA content to 1.8 μg/l. The contact time of F1 (3 days or 10 days) with the wine has no effect on the removal of the contaminant.

### Example 24

### Laboratory Scale Filtration of a White Beer in the Presence of Chitin-Glucan at a Dose of 200 g/hl

A batch of 10 liters of white beer is selected for filtration on a vertical candle filter in the presence of chitin-glucan at a dose of 200 g/hl. The chitin-glucan used is in the form of a powder with a particle size ranging from 50 to 90 μm.

In a first stage, a prelayer of chitin-glucan is formed on a vertical candle filter of aperture 30 μm. The chitin-glucan powder is suspended at 10% in water, and mixed for 1 hour before being deposited on the filter by circulation in a closed circuit at a flow rate of 20 hl.h⁻¹.m⁻².

In a second step, the circulation flow rate is reduced to 8 hl.h⁻¹.m⁻² and a water/beer mixture and then beer is circulated in an open circuit. The beer is placed in contact beforehand with chitin-glucan at a dose of 200 g/hl in the body feeding vat. The beer is filtered at a flow rate of 7 to 8 hl.h⁻¹.m⁻² on the chitin-glucan filtercake until all the volume has been filtered. The beer is then cooled to 8° C. and a sample is taken for analysis of the coagulable nitrogen and of the total polyphenols.

Results:

TABLE 51
Removal of the mycotoxins in the beer

| | | | % removed by filtration on |
|---|---|---|---|
| | Beer IN | Beer OUT | chitin-glucan |
| Coagulable | 378 mg/l | 162 mg/l | 57% |
| nitrogen | | | |
| Total polyphenols | 230 mg/l | 225 mg/l | 2% |

The chitin-glucan powder forms a filtercake that is sparingly compressible on the filter support used. The protein content, characterized by the content of coagulable nitrogen, of the beer filtered on this filtercake (OUT) is 57% less than the protein content of the control beer (IN). The total polyphenol content is unchanged.

### Example 25

### Clarification of Red Wine Musts with F1 at a Dose of 50 g/hl, Compared with a Control that has Undergone a Natural Decantation (10 l Tank)

A must from the CVC wine store (10 l tank) and a must from the CVSG wine store (10 l tank) were treated by adding fungal extract F1. This extract is added at the end of the alcoholic fermentation at a dose of 50 g/hl. The control must is clarified by natural decantation. The standard analyses of sugar, TAV, total acidity, volatile acidity, total SO₂, volatile SO₂, the pH and the turbidity are performed.

Results

TABLE 52
Variation of the turbidity of the musts and red wines obtained from
the must of the CVC wine store (10 l tank)

| | Turbidity | Variation |
|---|---|---|
| | (NTU) | (%) |
| Control starting must | 8277 | |
| Control wine | 1870 | 77.4% |
| Treated wine - F1 50 g/hl | 25 | 99.7% |

TABLE 53
Analytical characteristics of the musts and the red wines obtained
from the must of the CVC wine store (10 l tank)

| | | | T Ac. | | | | |
|---|---|---|---|---|---|---|---|
| | Sugar | TAV | (g/L | V Ac. (g/l | T SO₂ | L SO₂ | |
| | (g/L) | (% vol) | H₂SO₄) | H₂SO₄) | (mg/L) | (mg/L) | pH |
| Starting must | 198.0 | 0.05 | 2.82 | 0.05 | 18 | 3 | 3.43 |
| Control wine | 3.1 | 13.23 | 4.44 | 0.27 | 30 | 2 | 3.24 |
| Treated wine - F1 50 g/hl | 3.6 | 13.24 | 4.50 | 0.24 | 28 | 2 | 3.25 |

TABLE 54
Variation of the turbidity of the musts and red wines obtained from
the must of the CVSG wine store (10 l tank)

| | Turbidity (NTU) | Variation (%) |
|---|---|---|
| Control starting must | 3048 | |
| Control wine | 2332 | 23.4% |
| Treated wine - F1 50 g/hl | 749 | 75.4% |

TABLE 55
Analytical characteristics of the musts and red wines obtained from
the must of the CVSG wine store (10 l tank)

| | | | T Ac. | V Ac. | | | |
|---|---|---|---|---|---|---|---|
| | Sugar | TAV | (g/L | (g/L | T SO₂ | L SO₂ | |
| | (g/L) | (% vol) | H₂SO₄) | H₂SO₄) | (mg/L) | (mg/L) | pH |
| Control starting must | 195.0 | 0.05 | 2.82 | 0.05 | 18 | 3 | 3.43 |
| Control wine | 4.0 | 12.86 | 3.99 | 0.34 | 47 | 2 | 3.39 |
| Treated wine - F1 50 g/hl | 3.9 | 12.89 | 4.09 | 0.37 | 51 | 2 | 3.43 |

The treatment with the chitin-glucan F1 contributes towards improving the clarification of the red wines, without impairing the content of total phenolic compounds and tannins, compared with the control wine that has undergone a natural decantation.

### Example 26

### Clarification of Natural Sweet Wine Musts in a Poor State of Health with F1 at a Dose of 50 g/hl, Compared with a Control that has Undergone a Traditional Fining (Gelatin/Bentonite)

A natural sweet wine must in a poor state of health from vineyard DB (3 hl tank) was treated by adding fungal extract F1 before must clarification (before alcoholic fermentation), at a dose of 50 g/hl. The control wine underwent clarification with the traditional products, gelatin and bentonite.

Results

TABLE 56
Variation of the turbidity (3 hl tank, poor state of health)

| | Turbidity | |
|---|---|---|
| | (NTU) | Variation (%) |
| Control starting must | 333.5 | |
| Control wine | 19.3 | −94.2% |
| Treated wine - F1 50 g/hl | 16.6 | −95.1% |

TABLE 57
Analytical characteristics of the musts and natural sweet wines
obtained from the must from vineyard DB (3 hl tank, poor state of health)

| | | | T Ac. | V Ac. | | | |
|---|---|---|---|---|---|---|---|
| | Sugar | TAV | (g/L | (g/L | T SO₂ | L SO₂ | |
| | (g/L) | (% vol) | H₂SO₄) | H₂SO₄) | (mg/L) | (mg/L) | pH |
| Control starting must | 267.6 | 0.01 | 1.34 | 0.0 | 13 | 3 | 3.47 |
| Control wine | 106.0 | 14.86 | 2.76 | 0.43 | 1151 | 400 | 3.75 |
| Treated wine - F1 50 g/hl | 94.0 | 16.86 | 2.98 | 0.53 | 140 | 50 | 3.74 |

TABLE 58
Total phenolic compounds (TPC), tanins and colour intensity of the
musts and natural sweet wines obtained from the must from vineyard
DB (3 hl tank, poor state of health)

| | TPC | | |
|---|---|---|---|
| | (mg eq gallic | Tanins | OD |
| | acid/l) | (g/l) | 280 |
| Control starting must | 1017.2 | 0.28 | 0.1 |
| Control wine | 665.3 | 0.20 | 0.1 |
| Treated wine - F1 50 g/hl | 753.5 | 0.20 | 0.1 |

The addition of F1 gives rise to a decrease in the turbidity equivalent to that obtained after traditional treatment (gelatin/bentonite) without impairing the content of total phenolic compounds and tanins, or the colour intensity. At the level of the individual phenolic compounds, compared with the conventional treatment, the chitin-glucan binds B2 dimer and cyanidin.

### Example 27

### Clarification of Natural Sweet Wine Musts in a Good State of Health with F1 at a Dose of 50 g/hl, Compared with a Control that has Undergone a Conventional Fining (Gelatin/Bentonite)

A natural sweet wine must in a good state of health from vineyard DB (3 hl tank) was treated by adding fungal extract F1 before must clarification (before alcoholic fermentation), at a dose of 50 g/hl. The control wine underwent clarification with the conventional products gelatin and bentonite.

Results

TABLE 59
Variation of the turbidity (3 hl tank, good state of health)

| | Turbidity (NTU) | Variation (%) |
|---|---|---|
| Control starting must | 147.0 | |
| Control wine | 3.90 | −97.3% |
| Wine treated on must | 3.63 | −97.5% |
| clarification - F1 50 g/hl | | |
| Wine treated before mutage - | 2.50 | −98.3% |
| 50 g/hl | | |

TABLE 60
Analytical characteristics of the musts and natural sweet wines
obtained from the must from vineyard DB (3 hl tank, good state of health)

| | | | T Ac. | | | | |
|---|---|---|---|---|---|---|---|
| | Sugar | TAV | (g/L | V Ac. (g/L | T SO₂ | L SO₂ | |
| | (g/L) | (% vol) | H₂SO₄) | H₂SO₄) | (mg/L) | (mg/L) | pH |
| Control starting must | 212.0 | 0.01 | 2.09 | 0.0 | 18 | 3 | 3.44 |
| Control wine | 120.0 | 16.86 | 2.82 | 0.64 | 86 | 2 | 3.81 |
| Wine treated on must | 102.0 | 18.37 | 2.75 | 0.58 | 60 | 2 | 3.82 |
| clarification - F1 50 g/hl | | | | | | | |
| Wine treated before | 115.0 | 17.54 | 2.77 | 0.62 | 64 | 2 | 3.81 |
| mutage - F1 50 g/hl | | | | | | | |

TABLE 61
Total phenolic compounds (TPC), tannins and colour intensity of the
musts and natural sweet wines obtained from the must from vineyard
DB (3 hl tank, good state of health)

| | TPC | | |
|---|---|---|---|
| | (mg eq gallic | Tanins | OD |
| | acid/l) | (g/l) | 280 |
| Control starting must | 790.6 | 0.09 | 0.09 |
| Control wine | 291.3 | 0.05 | 0.07 |
| Treated wine - F1 50 g/hl | 280.0 | 0.06 | 0.07 |
| Wine treated before | 289.0 | 0.06 | 0.07 |
| mutage - F1 50 g/hl | | | |

The addition of F1 gives rise to a decrease in turbidity equivalent to that obtained after conventional treatment (gelatin/bentonite), without impairing the content of total phenolic compounds and tannins, or the colour intensity. Furthermore, the addition of F1 on must clarification or before mutage has no effect on the quality of the clarification.

### Example 28

### Clarification of Rosé Wine Musts with F1 at a Dose of 50 g/hl, Compared with a Control that has Undergone a Conventional Fining

A rosé wine must from the CV wine store (300 hl tank) was treated by adding fungal extract F1 after alcoholic fermentation, at a dose of 50 g/hl. The control underwent a conventional fining.

Results

TABLE 62
Variation of the turbidity (300 hl tank)

| | Turbidity |
|---|---|
| | (NTU) |
| Control tank must | 1710 |
| Control wine | 185 |
| Treated wine - F1 50 g/hl | 82 |

TABLE 63
Analytical characteristics of the musts and rose wines obtained from
the must from CV (300 hl tank)

| | | | T Ac. | | | | |
|---|---|---|---|---|---|---|---|
| | Sugar | TAV | (g/L | V Ac. (g/L | T SO₂ | L SO₂ | |
| | (g/L) | (% vol) | H₂SO₄) | H₂SO₄) | (mg/L) | (mg/L) | pH |
| Control starting must | 100 | 0.01 | 1.71 | 0.0 | 11 | 2 | 3.45 |
| Control wine | 1.9 | 13.78 | 2.98 | 0.26 | 64 | 2 | 3.55 |
| Treated must - F1 50 g/hl | 110 | 0.01 | 1.83 | 0.0 | 14 | 2 | 3.44 |
| Treated wine - F1 50 g/hl | 1.7 | 13.37 | 3.01 | 0.23 | 56 | 5 | 3.56 |

Treatment of the rosé wine must after alcoholic fermentation with F1 allows the rose wine musts to be clarified just as efficiently as by conventional fining. The content of total phenolic compounds, the anthocyan content and the optical density at 280 nm remain unchanged relative to the control.

At the level of the individual phenolic compounds, conventional fining gives rise to a decrease in the caffeic acid content, which is not the case when treatment is with the chitin-glucan.

### Example 29

### Removal of Mycotoxins in Naturally Contaminated Red Wines

A red wine from vineyard CV (426 hl tank) and a red wine from vineyard DB (3 hl tanks) containing ochratoxin A contents close to the maximum content recommended by the OIV (2 μg/l) were treated by adding fungal extract F1. Several treatment doses were tested: 129 g/hl, 300 g/hl, 400 g/hl and 500 g/hl. The fungal extract is left in contact with the wine for 3 days. The control wine undergoes no treatment.

Results

TABLE 64
Removal of mycotoxins in the CV red wine (426 hl tank)

| | | Variation |
|---|---|---|
| | OTA(μg/l) | (%) |
| Control | 1.7 | — |
| F1 - 129 g/hl | 1.4 | −17.6% |

TABLE 65
Analytical characteristics of the CV red wines (426 hl tank)

| | | | T Ac. | V Ac. | | | |
|---|---|---|---|---|---|---|---|
| | Sugar | TAV | (g/L | (g/L | T SO₂ | L SO₂ | |
| | (g/L) | (% vol) | H₂SO₄) | H₂SO₄) | (mg/L) | (mg/L) | pH |
| Control | 1.9 | 12.50 | 10.28 | 7.82 | 28 | 3 | 3.38 |
| F1 - | 2.1 | 12.57 | 9.69 | 7.24 | 28 | 3 | 3.37 |
| 129 g/hl | | | | | | | |

TABLE 66
Distribution of the colouring matter of the CV red wines (426 hl tank)

| | | | % brown |
|---|---|---|---|
| | % monomers | % red polymers | polymers |
| Control | 29.8 | 36.0 | 34.1 |
| F1 129 g/hl | 36.4 | 41.2 | 22.4 |

The F1 treatment at a dose of 129 g/hl gives rise to a 17.6% reduction in the OTA content of the red wine. The F1 treatment does not result in any change in the standard analytical parameters of wines. The contents of total phenolic compounds (approx. 2400 mg/l), tannins (approx. 3.3 g/l) and anthocyans (approx. 470 mg/l) and the colour intensity (OD 280 nm=0.57) on samples taken 2 days and 1 week after treatment are unchanged relative to the control. A slight modification of the phenolic material is noted, the chitin-glucan treatment brings about a decrease in the condensed polymer forms and an increase in the weakly polymerized polymer forms and in the free anthocyans.

TABLE 67
Removal of mycotoxins in the red wine from vineyard DB (3 hl tank)

| | OTA (μg/l) | Variation (%) |
|---|---|---|
| Control | 2.7 | — |
| F1 - 300 g/hl | 2.2 | −18.5% |
| F1 - 400 g/hl | 2.1 | −22.2% |
| F1 - 500 g/hl | 2.0 | −25.9% |

TABLE 68
Analytical characteristics of the red wines from vineyard DB
(3 hl tank)

| | | | T Ac. | V Ac. | | | |
|---|---|---|---|---|---|---|---|
| | Sugar | TAV | (g/L | (g/L | T SO₂ | L SO₂ | |
| | (g/L) | (% vol) | H₂SO₄) | H₂SO₄) | (mg/L) | (mg/L) | pH |
| Control | 1.5 | 13.66 | 3.47 | 0.59 | 25 | 2 | 3.55 |
| F1 - | 1.4 | 13.63 | 3.42 | 0.59 | 30 | 3 | 3.55 |
| 300 g/hl | | | | | | | |
| F1 - | 1.5 | 13.51 | 4.78 | 2.30 | 34 | 2 | 3.43 |
| 400 g/hl | | | | | | | |
| F1 - | 1.4 | 13.33 | 6.61 | 4.19 | 34 | 2 | 3.43 |
| 500 g/hl | | | | | | | |

The removal of the OTA in the red wine is dose dependent. The contents of total phenolic compounds (approx. 2070 mg/l) and tannins (approx. 2.43 g/l) and the colour intensity (OD 280 nm=0.47) after treatment are unchanged relative to the control. Irrespective of the dose of F1 treatment added to the wine (300 g/hl, 400 g/hr, 500 g/hl), this does not give rise to any changes in the standard analytical parameters of the wines.

### Example 30

### Small-Scale Removal of Mycotoxins in Naturally Contaminated Red Wines

A red wine from vineyard M (1 l bottles) containing OTA contents greater than or equal to the OIV recommendation was treated by adding fungal extract F1, at several doses and according to variable temperature and time conditions, in one or two additions. The fungal extract is left in contact with the wine for 3 days. The control wine undergoes no treatment.

TABLE 69
Analytical characteristics of the red wines from vineyard M
(1 l bottles)

| | | | T Ac. | | | | |
|---|---|---|---|---|---|---|---|
| | Sugar | TAV | (g/L | V Ac. (g/L | T SO₂ | L SO₂ | |
| | (g/L) | (% vol) | H₂SO₄) | H₂SO₄) | (mg/L) | (mg/L) | pH |
| Control | 1.1 | 12.97 | 5.36 | 2.60 | 13 | 2 | 3.69 |
| F1 - 200 g/hl | 1.2 | 13.09 | 3.65 | 0.56 | 6 | 2 | 3.73 |
| Ambient temperature, 3 | | | | | | | |
| days | | | | | | | |
| F1 - 300 g/hl | 1.1 | 13.14 | 3.64 | 0.55 | 6 | 2 | 3.73 |
| 0° C., 3 days | | | | | | | |
| F1 - 400 g/hl | 1.3 | 13.03 | 3.61 | 0.54 | 7 | 2 | 3.74 |
| Ambient temperature, 3 | | | | | | | |
| days | | | | | | | |
| F1 - 2 × 200 g/hl | 1.3 | 13.05 | 3.59 | 0.57 | 5 | 2 | 3.76 |
| Ambient temperature, 3 | | | | | | | |
| days | | | | | | | |
| F1 - 500 g/hl | 1.1 | 13.10 | 3.59 | 0.55 | 6 | 2 | 3.74 |
| Ambient temperature, 3 | | | | | | | |
| days | | | | | | | |
| F1 - 500 g/hl | 1.0 | 13.11 | 3.59 | 0.56 | 7 | 2 | 3.75 |
| 0° C., 3 days | | | | | | | |
| F1 - 300 g/hl | 1.0 | 12.89 | 3.62 | 0.59 | 7 | 2 | 3.73 |
| 10 days | | | | | | | |

TABLE 70
Removal of mycotoxins in the red wine from vineyard MD (1 l bottles)

| | OTA | Variation |
|---|---|---|
| | (μg/l) | (%) |
| Control | 3.0 | 12.97 |
| F1 - 200 g/hl | 2.3 | −23.3% |
| Ambient temperature, 3 | | |
| days | | |
| F1 - 300 g/hl | 2.0 | −33.3% |
| 0° C., 3 days | | |
| F1 - 400 g/hl | 1.9 | −36.7% |
| Ambient temperature, 3 | | |
| days | | |
| F1 - 2 × 200 g/hl | 1.8 | −40% |
| Ambient temperature, 3 | | |
| days | | |
| F1 - 500 g/hl | 2.0 | −33.3% |
| Ambient temperature, 3 | | |
| days | | |
| F1 - 500 g/hl | 1.9 | −36.7% |
| 0° C., 3 days | | |
| F1 - 300 g/hl | 2.0 | −33.3% |
| 10 days | | |

The removal is at least 23% with the F1 treatment at a dose of 200 g/hl. The contents of total phenolic compounds (approx. 2432 mg/l), tannins (approx. 2.95 g/l) and anthocyans (approx. 415 mg/l) and the colour intensity (OD 280 nm=0.56) after treatment are unchanged relative to the control.

The most efficient treatment protocol is the successive addition of 2 times 200 g/hl of F1, which makes it possible to reduce the OTA content to 1.8 μg/l. The period of time for which F1 is in contact (3 days or 10 days) with the wine has no effect on the removal of the contaminant.

### Example 31

### Fining of a Red Wine with the Chitin-Glucan at a Dose of 50 g/hl, Compared with a Control that has Undergone No Fining (340 hl Tank)

A finished wine (Cabernet Sauvignon) from the HR wine store was treated by adding chitin-glucan, on alcoholic fermentation, at a dose of 50 g/hl. The control wine underwent no fining.

TABLE 71
Analytical characteristics of the control wine

| | | | | V Ac. | | | | | | | Tannin |
|---|---|---|---|---|---|---|---|---|---|---|---|
| | Sugar | TAV | T Ac. | (g/l | T SO₂ | L SO₂ | | Malic acid | Lactic acid | TPC | index |
| | (g/l) | (% vol) | (g/l H₂SO₄) | H₂SO₄) | (mg/l) | (mg/l) | pH | (g/l) | (g/l) | (g/l) | (g/l) |
| Wine before fining | 1.1 | 13.8 | 2.9 | 0.2 | 52 | 7 | 3.71 | 1 | 1 | 80 | 0.13 |
| Wine after fining | 0 | 13.8 | 3.3 | 0.4 | 30 | 12 | 3.78 | 0 | 1.1 | 73 | 0.13 |

TABLE 72
Analytical characteristics of the wine treated with the chitin-glucan

| | | | | | | | | | | | Tannin |
|---|---|---|---|---|---|---|---|---|---|---|---|
| | Sugar | TAV | T Ac. | V Ac. | T SO₂ | L SO₂ | | Malic acid | Lactic acid | TPC | index |
| | (g/l) | (% vol) | (g/l H₂SO₄) | (g/l H₂SO₄) | (mg/l) | (mg/l) | pH | (g/l) | (g/l) | (g/l) | (g/l) |
| Wine before fining | 0 | 13.7 | 3.7 | 0.4 | 15 | 7 | 3.59 | 0.2 | 0.9 | 80 | 0.13 |
| Wine after fining | 0 | 13.4 | 3.6 | 0.4 | 30 | 15 | 3.56 | 0 | 0.7 | 61 | 0.13 |

The chitin-glucan treatment has no effect on the standard analytical parameters. The overall tannin content has not decreased, even after the treatment. The gustative and olfactory profiles of the wine treated with the chitin-glucan are similar to those of the untreated wine. The plant note is slightly more pronounced in the mouth and in the nose for the wine treated with chitin-glucan. The wine treated with chitin-glucan makes it possible to have tannins that blend in but are all the same full bodied. The treatment with chitin-glucan does not modify the analytical parameters or the sensory parameters, and it enables advantageous softening of the tannins.

### Example 32

### Fining of a Red Wine with the Chitin-Glucan at a Dose of 50 g/hl, Compared with a Control that has Undergone Fining with Bentonite at a Dose of 50 g/hl (300 hl Tank)

A finished wine (Merlot, thermovinified) from the CCG wine store, that was treated by adding chitin-glucan at the end of alcoholic fermentation, at a dose of 50 g/hl. The control wine underwent fining with bentonite at a dose of 50 g/hl.

TABLE 73
Analytical characteristics of the control wine

| | | | T Ac. | V Ac. | | | | | | | Tannin |
|---|---|---|---|---|---|---|---|---|---|---|---|
| | Sugar | TAV | (g/l | (g/l | T SO₂ | L SO₂ | | Malic acid | Lactic acid | TPC | index |
| | (g/l) | (% vol) | H₂SO₄) | H₂SO₄) | (mg/l) | (mg/l) | pH | (g/l) | (g/l) | (g/l) | (g/l) |
| Wine before | 0 | 12.6 | 3.3 | 0.35 | 35 | 72 | 3.46 | 0 | 1.3 | 40 | 1.6 |
| fining | | | | | | | | | | | |
| Wine after | 0 | 12.6 | 3.3 | 0.36 | 36 | 75 | 3.48 | 0 | 1.3 | 41 | 1.6 |
| fining | | | | | | | | | | | |

TABLE 74
Analytical characteristics of the wines treated with the chitin-glucan

| | | | | V Ac. | | | | | | | Tannin |
|---|---|---|---|---|---|---|---|---|---|---|---|
| | Sugar | TAV | T Ac. | (g/l | T SO₂ | L SO₂ | | Malic acid | Lactic | TPC | index |
| | (g/l) | (% vol) | (g/l H₂SO₄) | H₂SO₄) | (mg/l) | (mg/l) | pH | (g/l) | acid (g/l) | (g/l) | (g/l) |
| Wine before fining | 0 | 12.6 | 3.3 | 0.35 | 35 | 72 | 3.46 | 0 | 1.3 | 40 | 1.6 |
| Wine after fining | 0 | 12.6 | 3.3 | 0.36 | 34 | 74 | 3.49 | 0 | 1.4 | 40 | 1.6 |

In conclusion, all the results show that the treatment with chitin-glucan does not modify the analytical parameters but makes it possible to give a certain fruitiness to the nose.

### Example 33

### Fining of a Red Wine with the Chitin-Glucan at a Dose of 50 g/hl, Compared with a Control that has Undergone Fining with Bentonite at a Dose of 50 g/hl (300 hl Tank)

A finished wine (Cabernet Sauvignon, thermovinified) from the CCG wine store was treated by the addition of chitin-glucan at the end of the alcoholic fermentation at a dose of 50 g/hl. The control wine underwent fining with bentonite at a dose of 50 g/hl.

TABLE 75
Analytical characteristics of the control wine

| | | | T Ac. | | | | | | | | |
|---|---|---|---|---|---|---|---|---|---|---|---|
| | Sugar | TAV | (g/L | V Ac. (g/L | T SO₂ | L SO₂ | | Malic acid | Lactic | TPC | Tannin |
| | (g/L) | (% vol) | H₂SO₄) | H₂SO₄) | (mg/L) | (mg/L) | pH | (g/l) | acid (g/l) | (g/l) | index (g/l) |
| Wine before fining | 0 | 13 | 3.4 | 0.43 | 60 | 25 | 3.55 | 0 | 1.6 | 42 | 1.4 |
| Wine after fining | 0 | 13 | 3.5 | 0.42 | 62 | 22 | 3.59 | 0 | 1.7 | 43 | 1.4 |

TABLE 76
Analytical characteristics of the wine treated with the chitin-glucan

| | | | | V Ac. | | | | | | | |
|---|---|---|---|---|---|---|---|---|---|---|---|
| | Sugar | TAV | T Ac. | (g/l | T SO₂ | L SO₂ | | Malic acid | Lactic acid | TPC | Tannin index |
| | (g/L) | (% vol) | (g/l H₂SO₄) | H₂SO₄) | (mg/l) | (mg/l) | pH | (g/l) | (g/l) | (g/l) | (g/l) |
| Wine before fining | 0 | 12.6 | 3.3 | 0.43 | 60 | 25 | 3.6 | 0 | 1.6 | 44 | 1.4 |
| Wine after fining | 0 | 12.6 | 3.3 | 0.47 | 84 | 37 | 3.7 | 0 | 1.6 | 46 | 1.4 |

The treatment with the chitin-glucan has no effect on the conventional analytical parameters. It is noted that the overall tannin content did not decrease, even after the treatment with chitin-glucan. At the sensory level, the triangular test made it possible to demonstrate that no significant difference existed between the two wines. This is confirmed with the gustative and olfactory profiles of the wines. With this type of grape, the fining with chitin-glucan has the same effects as the treatment with bentonite.

### Example 34

### Clarification of a White Must with the Chitin-Glucan at a Dose of 30 or 50 g/hl, Compared with a Control that has Undergone Clarification with the Conventional Agents

Protocol

A white must (Sauvignon) from the PL wine store was treated by adding chitin-glucan (CG) at the time of must clarification, at a dose of 30 or 50 g/hl. The control wine underwent must clarification with the conventional products: 40 g/hl casein, 2 g/hl fish glue, 50 g/hl PVPP, 25 ml/hl silica gel, 50 ml/hl gelatin or 50 g/hl bentonite. In addition to the conventional sugar, TAV, total acidity, volatile acidity, total SO₂, volatile SO₂, pH and turbidity analyses, an analysis of the unstable protein content and also a study of the protein stability of the wines were carried out, according to the following protocols:

Unstable Protein Content by Capillary Electrophoresis

The analysis by capillary electrophoresis is carried out on the DUAL IMPACT apparatus from the company EUROPHOR, equipped with a 55 cm capillary column with an internal diameter of 75 μm.

0 ml of precentrifuged (5000 rpm, 10 minutes) wine are dialysed (cut-off threshold of 6 to 8000 Da) against 5 litres of 20 mM citric acid separating buffer at pH 2.5. The sample thus prepared is injected hydrodynamically for 1 second. The analytical conditions are the following:
Applied voltage: 10 kV
Analytical temperature: 25° C.
Detection of fractions leaving the column: 200 nm
   Under these analytical conditions, the proteins are positively charged and migrate to the anode. The proteins thus separated are divided up into 7 separate peaks.
   Must Protein Stability

0 ml of a wine prefiltered through a 0.45 μm membrane are subjected to a 30-minute heat treatment at 80° C. in a thermostated bath. The difference in turbidity is measured by nephelometry with a turbidimeter (Hach 2100 P) before heating, and after heating, after the heated wine has been cooled for 45 minutes. The difference in turbidity is proportional to the protein stability of the wine. A wine is considered stable if the difference in turbidity does not exceed 2 NTU.

Results

TABLE 77
Analytical characteristics of the musts

| | | | | | V Ac. | | | |
|---|---|---|---|---|---|---|---|---|
| | | Sugar | TAV | T Ac. | (g/l | T SO₂ | L SO₂ | |
| | Treatment | (g/l) | (% vol) | (g/l H₂SO₄) | H₂SO₄) | (mg/l) | (mg/l) | pH |
| Must | Control | 216 | 0 | 3.31 | 0.02 | 57 | 19 | 3.18 |
| before | | | | | | | | |
| must | | | | | | | | |
| clarification | | | | | | | | |
| Must after | Casein | 211 | 0 | 3.24 | 0.03 | 63 | 21 | 3.16 |
| must | Fish glue | 208 | 0 | 3.42 | 0.02 | 57 | 20 | 3.21 |
| clarification | PVPP | 212 | 0 | 3.29 | 0.02 | 62 | 21 | 3.19 |
| | Silica gel | 215 | 0 | 3.31 | 0.02 | 58 | 19 | 3.14 |
| | Gelatin | 217 | 0 | 3.32 | 0.02 | 56 | 18 | 3.18 |
| | Bentonite | 209 | 0 | 3.23 | 0.02 | 58 | 17 | 3.19 |
| | CG 30 g/hl | 213 | 0 | 3.31 | 0.02 | 57 | 13 | 3.17 |
| | CG 50 g/hl | 216 | 0 | 3.31 | 0.02 | 57 | 19 | 3.18 |

TABLE 78
Variation in the must turbidity

| | | Turbidity |
|---|---|---|
| | Treatment | (NTU) |
| Must before | | 293 |
| must | | |
| clarification | | |
| Must after | Casein | 7 |
| must | Fish glue | 5 |
| clarification | PVPP | 11 |
| | Silica gel | 41 |
| | Gelatin | 5 |
| | Bentonite | 36 |
| | CG 30 g/hl | 24 |
| | CG 50 g/hl | 14 |

TABLE 79
Variation in the unstable protein content and thermal stability of the
musts relative to the starting musts

| | Unstable proteins | | | | | Thermal |
|---|---|---|---|---|---|---|
| | Decrease | Decrease | Decrease | Decrease | Decrease | stability |
| | peak 1 | peak 2 | peaks 3-4 | peak 5 | peak 6 | after |
| Treatment | (%) | (%) | (%) | (%) | (%) | fermentation |
| Casein | 0 | 5 | 0 | 50 | 22 | Unstable |
| Fish glue | 27 | 8 | 11 | 8 | 0 | Unstable |
| PVPP | 0 | 12 | 3 | 0 | 59 | Unstable |
| Silica gel | 0 | 20 | 6 | 36 | 79 | Unstable |
| Gelatin | 38 | 0 | 0 | 65 | 65 | Unstable |
| Bentonite | 100 | 100 | 100 | 100 | 100 | Stable |
| CG 30 g/hl | 13 | 0 | 10 | 1 | 35 | Unstable |
| CG 50 g/hl | 34 | 21 | 18 | 39 | 61 | Unstable |

### Example 35

### Clarification of a White Must with the Chitin-Glucan at a Dose of 50 g/hl, Compared with a Control that has Undergone Clarification with a Pectolytic Enzyme

A white must (Chardonnay) from the CHR wine store was treated by the chitin-glucan at the time of must clarification, at a dose of 50 g/hl. The control wine underwent must clarification with a pectolytic enzyme (Novoclair speed).

TABLE 80
Analytical characteristics of the musts

| | | | | T Ac. | V Ac. | | | |
|---|---|---|---|---|---|---|---|---|
| | | Sugar | TAV | (g/L | (g/l | T SO₂ | L SO₂ | |
| Treatment | | (g/l) | (% vol) | H₂SO₄) | H₂SO₄) | (mg/l) | (mg/l) | pH |
| Novoclair | Must | 218 | 0 | 2.69 | 0.03 | 81 | 39 | 3.45 |
| speed | before | | | | | | | |
| | must | | | | | | | |
| | clarification | | | | | | | |
| | Must after | 186 | 0 | 2.53 | 0.03 | 68 | 33 | 3.39 |
| | must | | | | | | | |
| | clarification | | | | | | | |
| Chitin- | Must | 196 | 0 | 2.54 | 0.03 | 140 | 70 | 3.42 |
| glucan | before | | | | | | | |
| | must | | | | | | | |
| | clarification | | | | | | | |
| | Must after | 203 | 0 | 3.15 | 0.03 | 140 | 66 | 3.4 |
| | must | | | | | | | |
| | clarification | | | | | | | |

TABLE 81
Variation in the must turbidity

| | | Turbidity |
|---|---|---|
| Treatment | | (NTU) |
| Novoclair speed | Must before must clarification | 425 |
| | Must after must clarification | 29 |
| Chitin-glucan | Must before must clarification | 425 |
| | Must after must clarification | 70 |

TABLE 82
Variation in the unstable protein content, and thermal stability of the
musts relative to the musts before must clarification

| | | | | | | Thermal |
|---|---|---|---|---|---|---|
| | Unstable proteins | | | | | stability |
| | | Decrease | Decrease | Decrease | Decrease | after |
| | Decrease | peak 2 | peak 3-4 | peak 5 | peak 6 | alcoholic |
| Treatment | peak 1 (%) | (%) | (%) | (%) | (%) | fermentation |
| Novoclair | 0 | 72 | 37 | 55 | 0 | Unstable |
| speed | | | | | | |
| Chitin- | 0 | 0 | 28 | 28 | 3 | Unstable |
| glucan | | | | | | |

### Example 36

### Clarification of a White Must with the Chitin-Glucan at a Dose of 70 g/hl, Compared with a Control that has Undergone Clarification with a Pectolytic Enzyme

A white must (Terret) from the CHR wine store was treated by adding chitin-glucan at the time of must clarification, at a dose of 70 g/hl. The control wine underwent must clarification with a pectolytic enzyme (Ultrazym premium).

TABLE 83
Analytical characteristics of the musts

| | | | | T Ac. | | | | |
|---|---|---|---|---|---|---|---|---|
| | | Sugar | TAV | (g/L | V Ac. (g/L | T SO₂ | L SO₂ | |
| Treatment | | (g/L) | (% vol) | H₂SO₄) | H₂SO₄) | (mg/L) | (mg/L) | pH |
| Ultrazym | Must before | 168 | 0 | 3.75 | 0.04 | 73 | 37 | 3.28 |
| premium | must | | | | | | | |
| | clarification | | | | | | | |
| | Must after must | 176 | 0 | 3.92 | 0.04 | 79 | 39 | 3.32 |
| | clarification | | | | | | | |
| Chitin-glucan | Must before | 171 | 0 | 3.9 | 0.03 | 27 | 11 | 3.31 |
| | must | | | | | | | |
| | clarification | | | | | | | |
| | Must after must | 151 | 0 | 3.26 | 0.03 | 20 | 7 | 3.27 |
| | clarification | | | | | | | |

TABLE 84
Variation in the must turbidity

| Treatment | | Turbidity (NTU) |
|---|---|---|
| Ultrazym premium | Must before must | 1380 |
| | clarification | |
| | Must after must | 20 |
| | clarification | |
| Chitin-glucan | Must before must | 2130 |
| | clarification | |
| | Must after must | 84 |
| | clarification | |

TABLE 85
Variation in the unstable protein content, and thermal stability of the
musts relative to the musts before must clarification

| | | | | | | Thermal |
|---|---|---|---|---|---|---|
| | Unstable proteins | | | | | stability |
| | | Decrease | Decrease | Decrease | Decrease | after |
| | Decrease | peak 2 | peak 3-4 | peak 5 | peak 6 | alcoholic |
| Treatment | peak 1 (%) | (%) | (%) | (%) | (%) | fermentation |
| Ultrazym | 33 | 26 | 9 | 92 | 21 | Stable |
| premium | | | | | | |
| Chitin- | 83 | 65 | 81 | 75 | 100 | Stable |
| glucan | | | | | | |

The capacity of the chitin-glucan to clarify the musts, in comparison with conventional fining agents, is demonstrated by examples 3 to 6, the chitin-glucan being capable of rendering a must completely clarified. The chitin-glucan gives rise to a decrease in certain unstable proteins, generally in an insufficient manner to allow complete protein stability.

### Example 37

### Laboratory-Scale Filtration of a White Beer in the Presence of Chitin-Glucan at a Dose of 200 g/hl

Protocol

A batch of 10 litres of white beer is selected for filtration on a vertical candle filter in the presence of chitin-glucan, at a dose of 200 g/hl, in the form of a powder.

In a first step, a prelayer of chitin-glucan is formed on a vertical candle filter with an aperture of 30 μm. The chitin-glucan powder is suspended at 10% in water, and mixed for 1 hour before being deposited on the filter by closed-circuit circulation at a flow rate of 20 hl.h⁻¹.m⁻².

In a second step, the circulation flow rate is reduced to 8 hl.h⁻¹.m⁻², and a water/beer mixture and then beer is circulated in an open circuit. The beer is placed in contact beforehand with chitin-glucan at a dose of 200 g/hl in the body feeding vat. The beer is filtered at a flow rate of 7 to 8 hl.h⁻¹.m⁻² on the chitin-glucan filtercake until all the volume has been filtered, at ambient temperature. The beer is then placed in the cold at 8° C. and a sample is then taken for analysis of the coagulable nitrogen and of the total polyphenols.

Results

TABLE 86
Influence of the chitin-glucan on proteins (total nitrogen) and total
polyphenols

| | | | % removed by filtration on |
|---|---|---|---|
| | Beer IN | Beer OUT | chitin-glucan |
| Coagulable nitrogen | 378 mg/L | 162 mg/L | 57% |
| Total polyphenols | 230 mg/L | 225 mg/L | 2% |

The chitin-glucan powder forms a filtercake that is sparingly compressible on the filter support used. The protein content, characterized by the content of coagulable nitrogen, of the beer filtered on this filtercake (OUT) is 57% less than the protein content of the control beer (IN). The total polyphenol content is unchanged.

### Example 38

### Filtration of a “Biere De Garde” [French Country Ale] in the Presence of Chitin-Glucan

Protocol

A chitin-glucan powder is placed in contact with a “biere de garde” containing a yeast load of approximately 10⁵ cell/ml and a turbidity of 7 to 15 EBC (European Brewery Convention) units, according to the following protocol:

5 minutes with agitation

48 hours without agitation

The chitin-glucan doses used are 10, 20 and 30 g per hectolitre of beer.

After sedimentation of the chitin-glucan, the following characteristics are determined according to the EBC protocol:

Turbidity (EBC 9.29)

Protein content (Bradford calorimetric method)

Polyphenol content (EBC 9.11)

Spectrophotometric dosage of isohumulones (bitterness) (EBC 9.8)

Alcohol and real extract (EBC 9.2.1; 9.4 and 9.5)

Colour (EBC 9.6)

Content of volatile compounds and tasting

Results

The results of the physicochemical analyses are given in tables 52 and 53.

The chitin-glucan promotes natural sedimentation of the beer while kept. In fact, the turbidities are lower after 48 hours at 4° C. compared with the control beer filtered without adjuvant.

The chitin-glucan shows a low affinity for proteins. On the other hand, the biopolymer does not adsorb polyphenols, the other constituent of the colloidal cloudiness of the beer.

The other parameters are not affected by the chitin-glucan: bitterness, alcohol, extract, colour and organoleptic profile. In addition, the tasting did not demonstrate any significant difference between the samples.

TABLE 87
Various physicochemical determinations of the filtered beer,
after 5 min or 48 h of contact, with the chitin-glucan compared with a
beer filtered in the absence of chitin-glucan.

| | | 5 min | | | 48 h | | |
|---|---|---|---|---|---|---|---|
| Parameters | Control | 10 g/hl | 20 g/hl | 30 g/hl | 10 g/hl | 20 g/hl | 30 g/hl |
| Proteins (mg/l) | 180 | 165 | 152 | 177 | 175 | 175 | 160 |
| Polyphenols | 267 | 267 | 267 | 264 | 267 | 267 | 264 |
| (mg/ml) | | | | | | | |
| Turbidity at | | | | | | | |
| 90° (colloids) | 2.4 | 5.3 | 7.4 | 7.7 | 1.1 | 1.1 | 1.3 |
| 25° (particles) | 4.5 | 12.0 | 16.3 | 17.2 | 2.5 | 1.3 | 3.0 |
| Bitterness | 21.1 | 21.5 | 19.4 | 19.7 | 20.5 | 20.6 | 19.6 |
| (iso-alpha mg/l) | | | | | | | |
| Alcohol (% vol/vol) | 5.46 | 5.46 | 5.50 | 5.56 | 5.56 | 5.56 | 5.55 |
| Real extract (°P) | 2.42 | 2.41 | 2.41 | 2.41 | 2.41 | 2.41 | 2.41 |
| Colour (EBC) | 3.0 | 3.0 | 3.0 | 3.0 | 3.0 | 3.0 | 3.0 |

TABLE 88
Aromatic profile of the filtered beer, after 5 min or 48 h of contact,
with the chitin-glucan polymer compared with a beer filtered
in the absence of the polymer.

| | | 5 min | | | 48 h | | |
|---|---|---|---|---|---|---|---|
| Parameters | Control | 10 g/hl | 20 g/hl | 30 g/hl | 10 g/hl | 20 g/hl | 30 g/hl |
| Higher alcohols (ppm) | | | | | | | |
| Propanol | 9.3 | 13.7 | 14.7 | 10.1 | 13.1 | 14.5 | 10.3 |
| Isobutanol | 6.5 | 5.8 | 6.0 | 5.9 | 5.8 | 6.2 | 6.1 |
| Isoamyl alcohol | 37.3 | 36.3 | 36.3 | 36.1 | 37.6 | 37.6 | 36.4 |
| Esters (ppm) | | | | | | | |
| Ethyl acetate | 20.6 | 20.3 | 20.1 | 19.3 | 18.5 | 18.6 | 19.3 |
| Isoamyl acetate | 0.8 | 0.6 | 0.8 | 0.8 | 0.9 | 0.7 | 0.9 |
| Vicinal diketones (ppb) | | | | | | | |
| Diacetyl | 76 | 67 | 74 | 71 | 72 | 71 | 77 |
| 2,3-Pentanedione | 17 | 18 | 18 | 18 | 19 | 19 | 19 |

### Example 39

### Evaluation of the Effect of the Chitin-Glucan as an Ageing Tank Sedimentation Additive

Protocol

4 litres of green beer (released from ageing) are dispensed into four 1-litre stemmed glasses. 100, 200 and 300 mg of chitin-glucan powder are added to three of them, respectively.

Stirring is maintained in the four stemmed glasses for 4 minutes and they are subsequently placed at 4° C. Beer samples are taken regularly (at the surface of the liquid) and the turbidity is measured.

Results

TABLE 89
Turbidity at 90 and 25° of the beer being aged, treated with
the chitin-glucan (at the dose of 10, 20 or 30 g/hl)

| Samples | Control | 24 hrs | 53 hrs | 100 hrs |
|---|---|---|---|---|
| Control beer | 30/73 | 7.7/18.3 | 4.8/11.2 | 3.2/7.1 |
| Beer + 10 g/hl of chitin-glucan | 40/91 | 5.4/12.7 | 3.7/8.7 | 2.5/5.6 |
| Beer + 20 g/hl of chitin-glucan | 75/141 | 6.5/15.2 | 4.2/9.7 | 3.0/6.7 |
| Beer + 30 g/hl of chitin-glucan | 86/148 | 6.8/15.6 | 5.0/11.7 | 4.0/8.3 |

The results prove the effectiveness of the adjuvant as an aid to sedimentation/clarification of the beer being aged, from the first 24 hours of placing in contact onwards and from the concentration of 10 g/hl onwards.

The following examples relate to oral administration of a chitin-glucan copolymer

### Example 40

### Examples of Chitin-Glucan Copolymers Extracted and Purified from the Mycelium of Aspergillus niger (Extract F1)

To prepare the F1 chitin-glucan copolymer, a mass of 50 kg (dry weight) of wet Aspergillus niger mycelium is suspended in a solution of hydrochloric acid at a concentration of 1%, and then filtered. The solid matter is subsequently suspended in a solution of sodium hydroxide at 0.25%, and then filtered. The solid matter is washed 4 times with water and then dried. It is subsequently suspended in ethanol, and then filtered and dried. Approximately 20 kg of chitin-glucan (F1) are obtained.

The molecular characteristics and the composition of six batches of F1 chitin-glucan are given in Table 90.

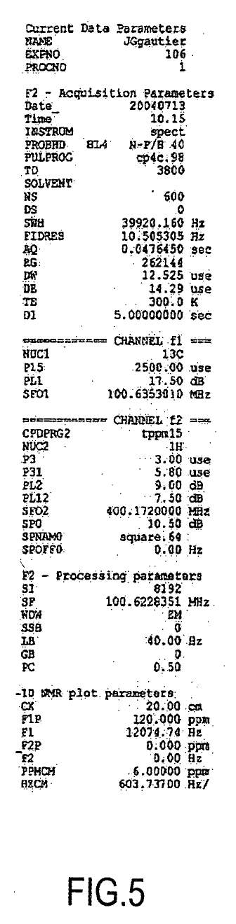

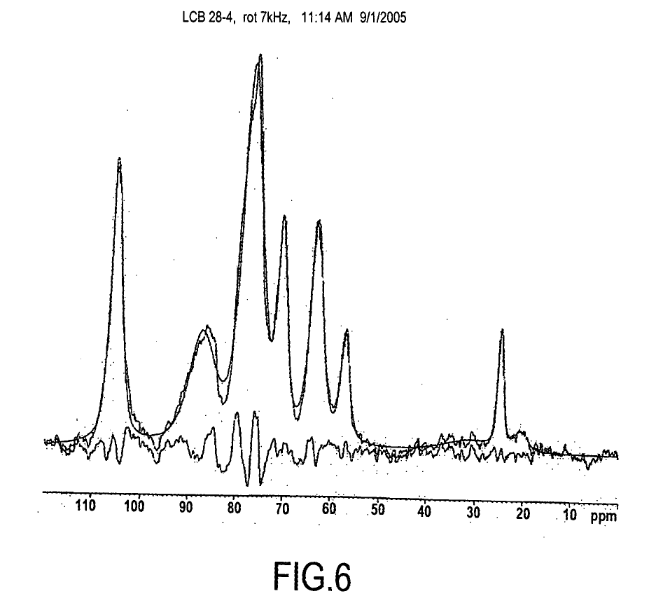

The chitin/glucan mass ratio is calculated from the solid-phase carbon 13 nuclear magnetic resonance (MNR) spectrum recorded under the conditions indicated in FIG. 5 according to the method briefly described below. The spectrum of the F1 chitin-glucan compound (batch 28) is shown in FIG. 6. The proportion of beta-glucan is determined from the area of the following four resonance bands: 104 ppm (carbon 1 of the chitin and of the beta-glucan), 23 ppm (CH₃ carbon of the chitin), 55 ppm (carbon 2 of the chitin) and 61 ppm (carbon 6 of the chitin and of the beta-glucan), taking pure chitin as reference. For example, the calculation can be carried out according to formula 1, where I′ is the area of the signals of the carbons, and where [ ]_{CG} indicates the value of the ratio for the chitin-glucan analysed and [ ]_{C} the value for the reference chitin. C1 is carbon 1 of the chitin and of the beta-glucan and C2 is carbon 2 of the chitin.

$$Glucan ( mol \% ) = \frac{[ \frac{{I}^{′} ( C 1 )}{{I}^{′} ( C 2 )} ] CG - [ \frac{{I}^{′} ( C 1 )}{{I}^{′} ( C 2 )} ] C}{[ \frac{{I}^{′} ( C 1 )}{{I}^{′} ( C 2 )} ] CG} ⨯ 100 \qquad ( 1 )$$

The chitin/glucan mass ratio of the six chitin-glucan batches is on average 39:61±2 (m/m).

The proportion of D-glucosamine (NGlc) units, expressed as mol % of the chitin part, can be estimated from the NMR spectrum, as described by Heux et al., (Heux L, Brugnerotto J, Desbrières J, Versali M F & Rinaudo M. (2000) Solid state NMR for determination of the degree of acetylation of chitin and chitosan. Biomacromolecules 1:746). The proportion of D-glucosamine units is determined by potentiometric titration with sodium hydroxide, in suspension in an excess of hydrochloric acid.

The microbiological quality of the chitin-glucan and the results of searching for pathogenic agents are given in Table 91.

TABLE 90
Molecular characteristics and composition of various batches of the
chitin-glucan copolymer (F1)

| | Chitin- | | | | | |
|---|---|---|---|---|---|---|
| | glucan | NGIc | | | | Heavy |
| | ratio | (titration) | Ash | Proteins | Lipids | metals |
| Batch | (m/m) | mol % | (%) | (%) | (%) | (ppm) |
| F1 batch | 36:64 ± 5* | 0 | 0.4 | 4.63 | 0 | <LQ** |
| 26 | | | | | | |
| F1 batch | 42:58 ± 7 | 0 | 1.3 | 3.54 | 0 | <LQ |
| 27 | | | | | | |
| F1 batch | 39:61 ± 7 | 0 | 1.5 | 2.51 | 2.09 | <LQ |
| 28 | | | | | | |
| F1 batch | 40:60 ± 6 | 0 | 1.7 | 3.05 | 0.06 | <LQ |
| 29 | | | | | | |
| F1 batch | 37:63 ± 1 | 0 | 1.9 | 1.54 | 1.24 | 8.7 |
| 30 | | | | | | |
| F1 batch | 40:60 ± 4 | 0 | 2.5 | 4.26 | 1.27 | <LQ |
| 31 | | | | | | |

*standard deviation over the result of 4 calculations of the chitin-glucan ratio;
**LQ: limit of sensitivity of the ion coupled plasma method of analysis (5.3 ppm)

TABLE 91
Microbiological quality of the F1 chitin-glucan (batch 26)

| | Number of |
|---|---|
| | microorganisms/g |
| Total mesophilic aerobic | 20 cfu/g |
| microorganisms | |
| Aerobic spores | <10 cfu/g |
| Yeasts and moulds | 20 cfu/g |
| Pathogenic agents | |
| Enterobacteriaceae | <10 cfu/g |
| Escherichia coil | <10 cfu/g |
| Staphylococcus coagulase+ | <10 cfu/g |
| Pseudomonas spp | 10 cfu/g |
| Salmonella spp | Absence |

It is thus understood from the above tables that the copolymer according to the present invention has a high degree of purity.

### Example 41

### Examples of Hydrolysates of the Chitin-Glucan Copolymer (F4)

In this example, the F4 chitin-glucan hydrolysates are obtained by basic hydrolysis of the F1 chitin-glucan copolymer at 100° C. or above, for at least 2 hours, in a solution of sodium hydroxide at a concentration of greater than 30%. The matter is washed with water several times, suspended in ethanol, and then filtered and dried. Approximately 5 kg of hydrolysate are obtained, starting from 20 kg of F1 chitin-glucan.

The molecular characteristics and the composition of some batches of F4 chitin-glucan hydrolysates are given in Table 92. The chitin-glucan ratio is calculated from the solid-phase carbon 13 nuclear magnetic resonance spectrum. The ratio is variable according to the method of hydrolysis used. The analytical conditions are identical to those used for F1.

TABLE 92
Molecular characteristics and composition (ash, proteins) of various
batches of chitin-glucan hydrolysates (F4)

| | Chitin-glucan | NGlc | | |
|---|---|---|---|---|
| | ratio | (titration) | Ash | Proteins |
| | (m/m) | mol % | % | % |
| F4 batch 1 | 26:74 ± 3 | 0.2 | ND | 0.1 |
| F4 batch 2 | 25:75 ± 2 | 0 | 1.2 | 8.9 |
| F4 batch 3 | 61:39 ± 3 | 0 | 5.3 | 3.9 |

* standard deviation over the result of 4 calculations of the chitin-glucan ratio

The copolymer according to the present invention clearly has a high degree of purity.

### Example 42

### Demonstration of the Anti-Atherosclerosis, Antioxidant, Blood-Cholesterol-Lowering and Blood-Lipid-Lowering Effect, after Oral Administration of Hydrolysed Chitin-Glucan

The animal model used is the hamster on an atherogenic diet. The hamsters are fed with a diet enriched in cholesterol and deficient in antioxidants (vitamin C, vitamin E and selenium), which brings about a dyslipidemia and arterial lesions similar to the lesions encountered in atheroma plaques in humans (Nistor A, Bulla A, Filip D A & Radu A (1987) The hyperlipidemic hamster as a model of experimental atherosclerosis. Atherosclerosis 68:159). The hamster represents a particularly advantageous animal model of atherosclerosis because of its lipid metabolism, the fact that it is easy to handle and the short period of time required to induce the lesions (from 8 to 12 weeks). It was chosen in order to study the effects of a chitin-glucan hydrolysate since it responds well to procedures aimed at decreasing cholesterol and atheroma (Kowala, M C, Nunnari, J J, Durham, S K & Nicolosi, R J (1991) Doxazosin and cholestyramine similarly decrease fatty streak formation in the aortic arch of hyperlipidemic hamsters. Atherosclerosis 91:3549).

In this example, two compounds, the characteristics of which are summarized in Table 93, were administered in the daily diet of the hamsters:

42.85 mg/kg/day of F4 hydrolysed chitin-glucan (i.e. 3 g/day for a man weighing 70 kg)

42.85 mg/kg/day of F7 chitosan of fungal origin (i.e. 3 g/day for a man weighing 70 kg)

The control group receives water by daily gavage in order to obtain the same experimental conditions as the other groups and to prevent any possible differences due to the stress of the gavage.

As soon as they are received, 28 Syrian golden hamsters weighing approximately 80 grams are placed in plastic cages, and divided up into three batches of eight animals in a room at 23° C., with a hygrometry of 70% and a photoperiod of 12 hours (12N/12D). The food consumption and their weights are measured every day. The groups receive the diet for which the composition is given in detail in Table 94.

TABLE 93
Characteristics of the F4 and F7 compounds

| | F4 | |
|---|---|---|
| | Hydrolysed | F7 |
| | chitin-glucan | Chitosan |
| Molecular mass | N/A | 10000 |
| Chitin (% of the chitin-glucan copolymer) | 25 | NA |
| Beta-glucan (% of the chitin-glucan copolymer) | 75 | 2 |
| Glucosamine (mol % of the chitin part) | 0 | 90 |
| Ash (%) | 1.2 | 1.3 |
| Proteins (%) | 8.9 | <1 |

TABLE 94
Composition of the hamster diet

| Ingredients | (g/KG) |
|---|---|
| Casein | 200 |
| DL-methionine | 3 |
| Wheat starch | 393 |
| Sucrose | 154 |
| Cellulose | 50 |
| Maize oil | 25 |
| Rapeseed oil | 25 |
| Mineral mixture¹ | 35 |
| Vitamin mixture² | 10 |
| Lard | 150 |
| Cholesterol | 5 |

¹The mineral mixture contains (mg/kg diet): CaHPO₄, 17200; KCl, 4000; NaCl, 4000; MgO, 420; MgSO₄, 2000; Fe₂O₃, 120; FeSO₄•7H₂O, 200; trace elements, 400 (MnSO₄•H₂O, 98; CuSO₄•5H₂O, 20; ZnSO₄•7H₂O, 80; CoSO₄•7H₂O, 0.16; KI, 0.32; starch, qs 40 g (per kg diet). This mixture lacks Na₂SeO₃.
²Vitamin mixture containing (mg/kg diet): retinal, 12; cholecalciferol, 0.125; thiamine, 40; riboflavin, 30; panthothenic acid, 140; pyridoxine, 20; inositol, 300; cyanocobalamine, 0.1; menadione, 80; nicotinic acid, 200; choline, 2720; folic acid, 10; p-aminobenzoic acid, 100, biotin, 0.6; starch, qs 20 g (per kg diet). This mixture lacks alpha-tocopherol and ascorbic acid.

Tissue sampling. After an experimental period of 12 weeks, the hamsters are placed under fasting conditions for 16 hours and are then anesthetized intraperitonally.
Plasma sampling. A blood sample is first taken by intracardiac puncture and then centrifuged at 3500×g for 10 minutes. The plasma is recovered, aliquoted, and then stored at −80° C. The total cholesterol, the HDL-cholesterol level, the LDL-cholesterol level, triglycerides, uric acid and urea will subsequently be determined on these plasmas.
Organ sampling. The liver is removed after perfusion with a solution of phosphate buffered saline (1 mM, pH 7.2) containing 1 mM calcium chloride and 0.27% of glucose. The aim of this is to remove the residue blood which could comprise an SOD and GSHPx activity. Said liver is subsequently dried, weighed, and then stored at −80° C. The glutathione peroxidase and superoxide dismutase activities will be determined on this organ.

The aorta is subsequently removed after fixing the vascular system with a 0.1 M sodium cacodylate buffer, pH 7.4, containing 2.5 mM calcium chloride, 2.5% of paraformaldehyde and 1.5% of glutaraldehyde. The aortic arch is then removed under a binocular lens, opened up longitudinally, pinned out on a piece of cork, and then immersed in the fixing solution and stored at 4° C. The atheromateous lesions caused by the diet may be observed after staining the aortic wall lipids.

Statistical Treatment of the Results

The values which will be presented in the following section correspond to the mean ±SEM (standard error of the mean) obtained on groups of 8 animals. The significance between the means was established by means of a one-variable ANOVA analysis using a Fischer test by means of the StatView 4.5 software (ABACCUS Concept, Inc). When the values bear an identical superscript letter, this means that these values are not significantly different for P<0.05.

Results

a—Determination of the lipid surface area. The surface area of the lipid deposits is determined using an optical microscope equipped with a photographic device, after histological staining according to the method described by Nunnari et al., (Nunnari J J, Zand T, Joris I & Majno G. (1989) Quantification of oil Red 0 staining of the aorta in hypercholesterolemic rats. Exp. Mol. Pathol. 51:1).

TABLE 95
Percentage of aortic surface area covered with aortic lipid deposits

| | Control | F4 | F7 |
|---|---|---|---|
| Percentage lipid | 14.79 ± 4.08^{a} | 0.35 ± 0.20^{b} | 0.57 ± 0.37^{b} |
| deposits | | | |

It is noted that the product according to the present invention is more effective than chitosan.
b—Plasma Assays
Several assays are carried out on the plasmas:

Biological panel (total cholesterol, triglycerides, HDL, LDL, uric acid, urea),

Assay for antioxidant capacity (TAS).

Determination of total plasma cholesterol. The analysis was carried out with kit No. 1489232 Roche/Hitachi, Roche Diagnostics. The assay is based on the technique described by Allain et al., (Allain C C, Poon L S, Chan C S, Richmond W & Fu P C. (1974) Enzymatic determination of total serum cholesterol, Clin. Chem. 20:470).

Determination of plasma triglycerides. The analysis was carried out with kit No. 1488872 Roche/Hitachi, Roche Diagnostics, according to the method developed by Wahlefeld & Bergmeyer (Wahlefeld A W & Bergmeyer H U (1974) in: Methods of enzymatic analysis Second Edition, New York Academic Press p. 1831).

Determination of plasma HDLs. The analysis was carried out with kit No. 1930672 Roche/Hitachi, Roche Diagnostics. The HDLs are separated according to the techniques described by Marz & Gross (März W & Gross W. (1986) Evaluation of a phosphotungstic acid/MgCl₂ precipitation and quantitative lipoprotein electrophoresis assay. Clin. Chim. Acta. 158:33).

Determination of plasma LDLs. The plasma LDL-cholesterol content is calculated by the difference between the total cholesterol content and the HDL-cholesterol content.

TABLE 96
Summary of the biochemical parameters

| | Control | F4 | F7 |
|---|---|---|---|
| Total cholesterol (g/l) | 3.10 ± 0.30a | 2.53 ± 0.21b | 2.37 ± 0.03b |
| Triglycerides (g/l) | 1.96 ± 0.55a | 1.04 ± 0.27bd | 0.60 ± 0.21bc |
| HDL (g/l) | 0.57 ± 0.16a | 2.01 ± 0.11bd | 1.84 ± 0.09bc |
| LDL (g/l) | 2.13 ± 0.2a | 0.24 ± 0.05b | 0.26 ± 0.09b |

It is noted that the products of the present invention have an effect equivalent to chitosan.
Determination of urea. The analysis was carried out with kit No. 1489364 Roche/Hitachi, Roche Diagnostics. This urea assay is based on a UV kinetic method described by Talke and Schubert (Talke H & Schubert G E. (1965) Enzymatische Harnstoffbest timmung im blut und serum im optischen test nach Warburg. Klin. Wochenschr. 43:174).

Determination of uric acid. The analysis was carried out with the COBAS INTEGRA 400/700/800 Uric Acid ver. 2 (UA2) Roche/Hitachi Kit, Roche Diagnostics. It is an enzymatic colorimetric assay according to the modified method of Town et al., (Town M H et al., (1985) J. Clin. Chem. Clin. Biochem. 23:591).

TABLE 97
Plasma enzymatic activities

| | Control | F4 | F7 |
|---|---|---|---|
| Uric acid | 255.00 ± 37.85^{a} | 100.75 ± 62.02^{bd} | 82.25 ± 21.70^{bc} |
| (μmol/l) | | | |
| Urea (mmol/l) | 14.87 ± 5.58^{a} | 4.00 ± 0.37^{b} | 5.25 ± 0.25^{b} |

It is noted that the plasma enzymatic activity of the products of the present invention is better than or equivalent to that of chitosan.

Assaying of plasma antioxidant capacity (TAS). This parameter is determined using the Randox No. NX2332 Kit (Laboratoire Randox), the method of which is based on the technique of Miller et al., (Miller N J, Rice-Evans C, Davies M J, Gopinathan V & Milner A. (1993) A novel method for measuring antioxidant capacity and its application to monitoring the antioxidant status in premature neonates, Clinical Science. 84:407).

TABLE 98
Plasma antioxidant adivity

| | Control | F4 | F7 |
|---|---|---|---|
| Antioxidant capacity | 1.03 ± 0.08^{abd} | 1.37 ± 0.07^{bce} | 0.89 ± 0.04^{de} |
| (mmol/l) | | | |

It is noted that the products of the present invention have a plasma antioxidant activity that is entirely surprising and not comparable to that of chitosan, which inhibits the plasma antioxidant activity.
c—Assaying Liver Enzyme Activities
Preparation of Cytosolic Fractions of Liver. The Liver Enzyme Activity is measured on cytosolic fractions; specifically, since the enzymes are in the cytosol, it is necessary to perform a two-step ultracentrifugation: a first centrifugation of the liver homogenates at 5% in 0.15M NaCl, at 8000×g for 20 minutes at 4° C., which makes it possible to recover a supernatant. The latter then undergoes a centrifugation at 105 000×g for one hour at 4° C. The cytosol is stored at −80° C. for the assays. The protein assay is carried out by the bicinchoninic acid method, according to Smith et al., (Smith P K et al., (1985) Measurement of protein using bicinchoninic acid. Anal. Biochem. 150:76).

Glutathione peroxidase activity (Se-GSHPx). The measurement of the activity of this enzyme is based on the ability of the sample to catalyse the oxidation of glutathione by aqueous hydrogen peroxide, according to the method of Wendel (Wendel A. (1981) Glutathione peroxydase. Methods in enzymology, 77:327).

TABLE 99
Specific activity of liver Se-GSHPx

| | Control | F4 | F7 |
|---|---|---|---|
| Se-GSHPx | 109.12 ± 88.94^{a} | 134.04 ± 1.49^{a} | 142.85 ± 54.60^{a} |
| (U/mg proteins) | | | |

It is noted that the products of the present invention have a liver enzyme activity equivalent to that of chitosan.

Superoxide dismutase activity (SOD). The activity of SOD is determined with the SOD 525 Kit (Tebu-Bio) according to the method of Paoletti and Mocali (Paoletti F & Mocali A. (1990) Determination of superoxide dismutase activity by purely chemical system based on NADCPJH oxidation, Methods Enzymology. 186:209).

TABLE 100
Specific activity of liver SOD

| | Control | F4 | F7 |
|---|---|---|---|
| SOD | 0.58 ± 0.56^{a} | 0.03 ± 0.02^{bd} | 0.04 ± 0.01^{cd} |
| (U/mg proteins) | | | |

It is noted that the products of the invention have an activity on superoxide dismutase comparable to that of chitosan.

To summarize the results, Tables 101 and 102 bring together the variations in the various parameters expressed as % relative to the control group.

TABLE 101
Variations in the surface area of aortic lipid deposits and in the liver
enzyme activities expressed as % relative to the control group

| | Se-GSHPx | SOD | Percentage lipid |
|---|---|---|---|
| | (U/mg proteins) | (U/mg proteins) | deposits |
| F4 | +22.8% | −94.8% | −97.6% |
| F7 | +30.9% | −93.1% | −96.1% |

TABLE 102
Variations in the various biochemical parameters expressed as %,
relative to the control group.

| | Total | | | | Antioxidant | Uric | |
|---|---|---|---|---|---|---|---|
| | cholesterol | Triglycerides | HDL | LDL | capacity | acid | Urea |
| | (g/l) | (g/l) | (g/l) | (g/l) | (mmol/l) | (μmol/l) | (mmol/l) |
| F4 | −18.4% | −46.9% | +252.6% | −88.7% | +33.1% | −60.5% | −73.1% |
| F7 | −23.5% | −69.4% | +222.8% | −87.8% | −13.6% | −67.7% | −64.7% |

Total cholesterol, triglycerides, HDL-cholesterol and LDL-cholesterol are biochemical parameters which make it possible to note the blood-cholesterol-lowering effects of the hydrolysed chitin-glucan and of chitosan. In fact, the decrease in total cholesterol and in LDL-cholesterol (significant decrease by a factor of 10) compared to the control group make it possible to confirm the effectiveness of the hydrolysed chitin-glucan and of chitosan in preventing the development of atheroma plaque and, consequently, on cardiovascular diseases.

The increase in HDL-cholesterol is spectacular. It reflects the fact that the efflux of cholesterol from the peripheral organs to the liver is promoted, thus decreasing the risk of deposits and of oxidation of particles rich in cholesterol in the organs and the arteries. The treatments based on F4 and F7 make it possible to invert the HDL/LDL levels compared to the control.

The percentage of the surface area of the aortic arch covered with lipid deposits and foam cells is a direct indicator of the development of atheromateous lesions. Compared with the controls, the two types of treatment very significantly reduce this percentage, with a level of effectiveness that is virtually similar, around 97%. Now, compounds reputed to be very effective in reducing lipid deposits, such as wine polyphenols, exhibit a reduction in surface area of the aortic arch covered with lipid deposits of 70 to 90% according to the molecules studied with the same hamster molecule on an atherogenic diet (Auger R et al., J. Agric. Food Chem. (2005) 53:9823; Auger R et al., (2005) J. Agric. Food Chem. 53:2015; Auger R et al., (2004) J. Agric. Food Chem. 52:5297).

Only F4 increases the plasma antioxidant capacity, by 33.1% compared with the control group. As regards the activities of the liver enzymes involved in the antioxidant system, it is observed that the selenium-dependent glutathione peroxidase activity is increased, and that the SOD activity is greatly decreased by the treatments with F4 and F7 compared with the control group.

A very large decrease (60 to 70%) in uric acid and also in urea in the plasma can also be noted for two groups treated with F4 and F7. The decreases observed reflect a greater elimination of uric acid and of urea by the kidneys, which decreases the risk of hyperuricemia, a parameter which is known to promote atheroma plaque development.

The considerable improvement in the lipid profile and in the associated parameters for the animal model used makes it possible to conclude that the regular consumption of hydrolysed chitin-glucan is of benefit in preventing atherosclerosis and, by extension, obesity, with effects that are comparable to, or even better than, those induced by the consumption of chitosan.

### Example 43

### Effect of the Oral Administration of Chitin-Glucan on the Gastrointestinal Tract, Carbohydrate Profile and Lipid Profile in the Rat

Protocol

The model used is the normal rat given a standard diet and drink enriched in fructose (21%), which promotes hepatic steatosis, via rapid metabolisation of the fructose by the liver, resulting in hypertriglyceridemia and hyperinsulinemia and a reduction in the action of insulin in skeletal muscle and the liver.

The chitin-glucan (table 103) is administered in the daily diet of the rats at a rate of 10%, which is a dose capable of generating an acute effect in the animal. The short-term and long-term effects are studied. The control group receives a standard diet enriched in fructose (table 104). 10 male Wistar rats weighing approximately 100-125 g are divided up into cages in two groups (control/treated) in a room at 23° C., with a hygrometry of 70% and with a photoperiod of 12 hours (12N/12D).

Food consumption and weight change are measured once a week. The production of faeces over 24 h and the water content of the latter are estimated after 2 weeks of treatment. After the acclimatization period (1 week), during which the animals receive a standard diet not enriched in fructose, an evaluation of gastric emptying and of the glycaemic response in the presence of the chitin-glucan is carried out. The rats are given no food for 18 hours. The treated group receives a solution of glucose-paracetamol-chitin-glucan (10%) by gavage, while the control group receives a glucose-paracetamol solution. Blood samples are taken for 2 hours (blood glucose level, blood insulin level, paracetamol). Similarly, an evaluation of gastric emptying and of the glycaemic response in the absence of chitin-glucan is carried out after 3 weeks of treatment. In this case, the rats are given no food for 18 h and a glucose-paracetamol solution is administered by gavage for 2 days. Blood samples are taken for 2 hours (blood glucose level, blood insulin level, paracetamol). Over the course of the third week of treatment, the rats are given no food for 18 hours. Subsequently, an evaluation of the orofecal transit time is carried out. Diets coloured with a solution of carmine red are given to the rats. The time for the first coloured faeces to appear is determined.

After 4 weeks of treatment, the rats are anaesthetized and a laparotomy is performed. The blood from the portal vein and the vena cava is centrifuged in order to recover the serum and/or the plasma. The lipid (total cholesterol, HDL-cholesterol, LDL-cholesterol) and carbohydrate (glucose, insulin) profiles are analysed using the latter. The various organs are removed, weighed and conserved in order to estimate the caecal fermentation (caecal proliferation, caecal pH, short-chain carboxylic acid content), the lipid content of the liver and the faeces, and the adipose mass (weight of epidydimal and visceral adipose tissues).

Results (Table 105)

The results show that a regular consumption of chitin-glucan promotes a balanced intestinal transit and a balanced homeostasis of lipid and carbohydrate profiles in a rat model. It is seen that the “fibre effect” has a preventive effect on parameters directly related to metabolic diseases such as hypercholesterolaemia, diabetes or, by extension, metabolic syndrome and obesity. In fact, the fibre reduces the caloric density of food (number of calories per 100 g), significantly slowing down gastric emptying and food digestion, and significantly reduce insulin secretion. These effects together result in a spontaneous reduction in calorie consumption and in the feeling of being hungry.

The chitin-glucan induces fermentation at the caecal level. This fermentation is accompanied by short-chain fatty acid production which is significantly higher in the group receiving the chitin-glucan compared with the control group, by influencing epithelial cell homeostasis. This thus contributes to reducing the risks of atrophy, to stimulating the recovery of a damaged intestinal epithelium, and/or to inhibiting cell hyperproliferation.

TABLE 103
Molecular characteristics and composition of the chitin-glucan
copolymer

| Chitin-glucan | | | | |
|---|---|---|---|---|
| ratio | Ash | Proteins | Lipids | Heavy metals |
| 35/65 | 2.3 | 7.71 | 1.5 | <20 |

TABLE 104
Rat food composition (standard AO4 diet, UAR, France)

| | % |
|---|---|
| Proteins | 19.3 |
| Carbohydrates: | 70.4 |
| Cellulose | 5 |
| Starch | 38 |
| Sucrose | 3 |
| Other undigestible | 8 |
| substances | |
| Lipids | 3 |
| Mineral/vitamin mixtures | 7.3 |

TABLE 105
Variation in the biochemical and physiological parameters of the
groups of rats having consumed the chitin-glucan,
compared with the control group

| | Chitin-glucan |
|---|---|
| | (CG) |
| Weight change | [symbol] |
| Food consumption | [symbol] |
| Blood glucose level | ≅ |
| Blood insulin level | [symbol] |
| Adipose mass | [symbol] |
| Faecal production over 24 h | [symbol] |
| Gastric emptying | [symbol] |
| Orofaecal transit | [symbol] |
| Caecal fermentation | [symbol] |

[symbol]

: significantly lower than the value for the control group (p < 0.05); [symbol]
: significantly higher than the value for the control group (p < 0.05);
≅ significantly unchanged (p < 0.05)

### Example 44

### Water Absorption Capacity of Chitin-Glucan

In order to mimic the behaviour of the chitin-glucan in the gastrointestinal tract, powdered chitin-glucan is brought into contact with water, NaCl and aqueous solutions at various pHs. After a contact time of 12 hours with stirring, the chitin-glucan is separated by centrifugation and the wet mass is determined.

TABLE 106
Swelling capacity of chitin-glucan in various media (g of water
absorbed per 100 g of dry chitin-glucan)

| Water | 5% NaCl | pH 3 | pH 5 | pH 7 | pH 9 |
|---|---|---|---|---|---|
| 7 | 7 | 7 | 6 | 7 | 7 |

Results

It is seen from this example that the chitin-glucan is capable of absorbing approximately 6 times its mass of water or of an aqueous medium, which is of the same order of magnitude as the degree of swelling of the known commercial insoluble dietary fibres, for instance hemicellulose and pectin (4 to 8 times their mass of water).

### Example 45

### Demonstration of the Anti-Atherosclerosis, Antioxidant, Blood-Cholesterol-Lowering and Blood-Lipid-Lowering Effect after Oral Administration of Chitin-Glucan in the Hamster

Protocol

The model used is the same as that presented in example 42. In this example, two compounds, the characteristics of which are summarized in table 107, were administered in the daily diet of hamsters:

CG2 group: 42.85 mg/kg/day of chitin-glucan (i.e. 2 g/day for a man weighing 70 kg)
CG1.5 group: 21.43 mg/kg/day of chitin-glucan (i.e. 1.5 g/day for a man weighing 70 kg)
Cs2 group: 28.57 mg/kg/day of chitosan (i.e. 2 g/day for a man weighing 70 kg)

The control group receives water by daily gavage in order to obtain the same experimental conditions.

TABLE 107
Characteristics of the chitin-glucan and chitosan compounds

| | Chitin-glucan | Chitosan |
|---|---|---|
| | (CG) | (Cs) |
| Molecular mass | N/A | 29,000 |
| Chitin | 35 | N/A |
| (% of the chitin-glucan | | |
| copolymer) | | |
| Beta-glucan | 65 | 2.1 |
| (mol % of the chitin component) | | |
| Glucosamine | 0 | 89 |
| (mol % of the chitin component) | | |
| Ash (%) | 2.3 | 2.66 |
| Protein (%) | 7.7 | 0.31 |

As soon as they are received, 48 Syrian golden hamsters weighing approximately 100 g are placed under the same experimental conditions of example 42. The food consumption and their weights are measured every day. The groups receive the same food as that presented in example 42 (table 108).
Results

The samplings, the statistical analyses and the analytical methods are the same as those presented in example 42. All the results are presented in table 108.

TABLE 108
Variation in the biochemical parameters of the groups of hamsters
having consumed the chitin-glucan and the chitosan,
compared with the control group

| | CG2 group | CG1.5 group | Cs2 group |
|---|---|---|---|
| | (2 g/day) | (1.5 g/day) | (2 g/day) |
| Total cholesterol | [symbol] | [symbol] | [symbol] |
| Triglycerides | [symbol] | [symbol] | [symbol] |
| HDL-cholesterol | [symbol] | [symbol] | [symbol] |
| LDL-cholesterol | [symbol] | [symbol] | [symbol] |
| Antioxidant capacity | [symbol] | [symbol] | [symbol] |
| Uric acid | [symbol] | [symbol] | [symbol] |
| Urea | [symbol] | [symbol] | [symbol] |
| Se GSHPx | [symbol] | [symbol] | [symbol] |
| SOD | [symbol] | [symbol] | [symbol] |
| Percentage of lipid deposits | [symbol] | [symbol] | [symbol] |

[symbol]

: significantly lower than the value for the control group (p < 0.05); [symbol]
: significantly higher than the value for the control group (p < 0.05)

It is seen from this example that the chitin-glucan brings about the same variations in the biochemical parameters of the rats on an atherogenic diet as the products studied in example 42. The chitin-glucan considerably improves the lipid profile and the associated parameters, at the two doses studied. Regular consumption of chitin-glucan is therefore beneficial in the prevention of atherosclerosis and, by extension, of related pathologies.

What is claimed is:

1. A method for isolating and purifying cell wall derivatives from a fungal biomass comprising the subsequent steps of suspending the biomass in an acidic solution and remove the acid soluble fractions, followed by suspending the acid-insoluble fraction in an alkaline solution and removing the alkali-soluble fraction, followed by further purifying and drying the alkali-insoluble fraction.

2. The method according to claim 1, wherein said method is comprising the following subsequent steps:
a) Optionally suspending the biomass in an acidic solution and remove the acid soluble product,
b) Suspending the acid-insoluble fraction in an alkaline solution and removing the alkali-soluble product,
c) Purifying the alkali-insoluble product by further treatment with water,
d) Drying the water-insoluble product
e) Optionally purifying the dried product by treatment in organic solvent,
f) Optionally drying the organic-insoluble product.

3. The method according to claim 1, wherein said basic solution comprises a concentration lower than 10% (w/v).

4. The method according to claim 1, characterized in that said biomass is selected from the group comprising Zygomycetes, Basidiomycetes, Ascomycetes and Deuteromycetes and/or mixtures thereof.

5. A chitin-glucan copolymer obtainable by the method according to claim 1.

6. A chitin polymers of fungi origin, said polymer containing more than 80% chitin.

7. A chitin-rich chitin-glucan copolymers obtainable by the method according to claim 1.

8. A method comprising the use of chitin polymers, chitin-glucan copolymers, chitin-rich chitin-glucan copolymers or chitosan polymers according to claim 1 in medical, pharmaceutical, agricultural, nutraceutical, food, textile, cosmetic, industrial, or environmental applications.

9. A method for treating a food-grade liquid of plant origin, comprising placing a food-grade liquid of plant origin in contact with at least one technological additive, said technological additive being a fungal extract predominantly comprising at least one nonionic polysaccharide, said nonionic polysaccharide predominantly comprising at least one chitin-glucan copolymer.

10. The method as claimed in claim 9, wherein said chitin-glucan copolymer has a chitin/glucan ratio of between 95:5 and 5:95 (m/m).

11. The method as claimed claim 9, wherein said method is for the partial or total removal of undesirable compounds causing instability of the food-grade liquid or causing health risks by drinking said food-grade liquid.

12. The method as claimed in claim 9, wherein said method is for improving the quality of the food-grade liquid, said method comprising removing the undesirable compounds which are selected from the group consisting of colloids causing instability, colloids causing cloudiness, colloids that give poor-quality organoleptic properties, proteins, metals, heavy metals, iron, cadmium and lead, residual pesticides, fungicides, insecticides, herbicides, toxins, mycotoxins, and bacterial endotoxins.

13. The method as claimed in claim 9, wherein said method is for treating finished food-grade liquids or for clarifying a food-grade liquid of plant origin.

14. The method as claimed in claim 9, wherein said food-grade liquid of plant origin is chosen from a fermented beverage and a fruit juice.

15. The method as claimed in claim 9, wherein said fermented beverage is a wine.

16. The method as claimed in claim 9, wherein said fermented beverage is a beer.

17. A method comprising orally administering to a mammal at least one polysaccharide of fungal origin comprising predominantly a chitin-glucan copolymer or a hydrolysate thereof.

18. The method of claim 17, wherein Aspergillus niger is used as fungal origin.

19. The method of claim 17, wherein the chitin-glucan hydrolysate comprises beta-glucans and has a ratio of chitin to beta-glucans of between 60:40 and 15:85 (m/m).

20. The method of claim 17, wherein oral administration is for obtaining an effect selected from the group consisting of an antioxidant, blood-cholesterol-lowering or blood-lipid-lowering effect, a stimulatory effect on the immune system, a hypoglycaemic effect a satiety effect, an effect which improves food transit, and an effect consisting in preventing, or treating, or combating a pathology selected from the group consisting of dyslipidemia, atherosclerosis, obesity, an obesity-related disease, a cardiovascular disease, metabolic syndrome, diabetes, and hyperuricemia.

21. A food supplement composition comprising, as active ingredient, at least one polysaccharide, or one extract of fungal origin, comprising a chitin-betaglucan copolymer comprising a ratio of chitin to beta-glucans of between 50:50 and 10:90.

22. A pharmaceutical composition comprising, as active ingredient, at least one polysaccharide or one extract of fungal origin, comprising a chitin-betaglucan copolymer comprising a ratio of chitin to beta-glucans of between 50:50 and 10:90.
